(19) United States
(12) Patent Application Publication
(10) Pub. No.: US 2025/0108088 A1
(43) Pub. Date: Apr. 3, 2025

(54) REGENERATIVE PEPTIDES AND METHODS FOR USE THEREOF

(71) Applicant: Matice BioSciences, Inc., Cambridge, MA (US)

(72) Inventors: Mark Theodore C. MENESES, Cambridge, MA (US); Jonathan S. WOLFSON, Portola Valley, CA (US); Jessica L. WHITED, Belmont, MA (US); Brandy M. HOFFMAN, Chicago, IL (US); Peter LICARI, Fremont, CA (US)

(21) Appl. No.: 18/903,801

(22) Filed: Oct. 1, 2024

Related U.S. Application Data
(60) Provisional application No. 63/696,295, filed on Sep. 18, 2024.
(60) Provisional application No. 63/587,325, filed on Oct. 2, 2023.

(51) Int. Cl. A61K 38/10; A61P 17/00; C07K 9/00; G01N 33/50

(52) U.S. Cl. CPC A61K 38/10; A61P 17/00; C07K 9/00; G01N 33/5044

(57) ABSTRACT

Provided herein are peptide compositions derived from regenerative species regenerative proteins, and methods of making and use thereof, that seek to reduce fibrosis. The disclosed peptide formulations and compositions may be used to reduce fibrosis resulting from any disease or disorder, e.g., fibrosis associated with tissue inflammation, wounds, disease, or infection.

### CROSS-REFERENCE TO RELATED APPLICATIONS

This application claims priority to U.S. provisional patent application No. 63/587,325, filed on Oct. 2, 2023, and U.S. provisional patent application No. 63/696,295 filed on Sep. 18, 2024, the contents of each of which are incorporated herein by reference in their entireties.

### REFERENCE TO AN ELECTRONIC SEQUENCE LISTING

The contents of the electronic sequence listing (MTCE_002_02US_SeqList_ST26.xml; Size: 86,453 bytes; and Date of Creation: Sep. 25, 2024) are herein incorporated by reference in their entirety.

### BACKGROUND

Fibrosis is a thickening or hardening of tissue that may affect any major organ, including but not limited to the skin, lungs, heart, liver, and kidneys. Fibrosis can lower both the quality of life and life expectancy of a patient and is implicated in an estimated 30-40% of deaths annually worldwide.

In most mammals, fibrosis is caused by collagen and fibronectin deposited differentially in response to tissue damage from inflammation, wounds, disease, or infection. In contrast, in regenerative species, and in humans before birth, fibrosis does not occur in response to tissue damage, and the tissue generated in response to damage is functional and indistinguishable from native tissue. Axolotl salamanders, for example, heal restitutio ad integrum, in which tissues, including limbs that are lost, are completely re-grown to their native state without fibrotic tissue. Humans too, can regenerate tissues completely as embryos and fetuses without detectible fibrosis.

Currently there are no effective treatments for fibrosis; however, translating the tissue regeneration capacities of regenerative species into anti-fibrotic treatments would greatly improve human mortality and quality of life. Thus, there is a clear need for the development of anti-fibrotic treatments.

### SUMMARY

Provided herein are peptide compositions derived from regenerative species proteins, and methods of making and use thereof, that seek to reduce fibrosis as anti-fibrotic treatments. Also contemplated herein are use of the peptides derived from regenerative species proteins in wound healing treatments.

Contemplated herein are various methods for selecting, modifying, and making anti-fibrotic peptides derived from a regenerative species regenerative protein. In some aspects, provided herein is a method of synthesizing a peptide for reducing fibrosis comprising:
i) selecting a peptide sequence from a regenerative protein sequence from a regenerative species; and
ii) modifying the peptide sequence to improve one or more of cross-species homology, tissue penetration, peptide stability, and/or affinity to human proteases; and
iii) synthesizing the peptide;
wherein the peptide regulates one or more of: myofibroblast formation, bacterial infection, inflammation, cell migration, extracellular matrix production, keratin production or cell proliferation.

The peptides of the instant disclosure may be derived from regenerative proteins from various regenerative species. In some aspects, the regenerative protein is an extracellular protein. In some aspects, the regenerative protein is an intracellular protein.

In some aspects, the peptides are modified to improve cross-species homology. In some aspects, the modifying to improve cross-species homology improves the sequence homology between sequences of a regenerative species and a human. In some aspects, the modifying to improve cross-species homology improves the sequence homology between sequences of two or more regenerative species. In some aspects, the regenerative species is selected from axolotl, spiny mouse, starfish, planarian, and zebrafish. In some aspects, the modifying to improve cross-species homology includes adding one or more amino acid residues from a homologous sequence at the C-terminus or N-terminus of the peptide fragment or modifying the amino acid sequence of the peptide to increase the cross-species sequence homology by at least 20%.

In some aspects, the extracellular matrix production regulated by the peptide comprises one or more of collagen, elastin, or hyaluronic acid.

In some aspects, the peptides are modified to improve tissue penetration. In some aspects, the modifying to improve tissue penetration of the peptide includes adding a peptide sequence of less than 40 amino acids, wherein the peptide sequence has a positive net charge and is amphiphilic. In some aspects, the modifying to improve tissue penetration of the peptide includes adding the peptide sequence of GLRKRLRKFRNK (SEQ ID NO: 1). In some aspects, the modifying improves tissue penetration of the peptide for an anti-fibrotic treatment by about 20%, by about 30%, by about 40%, by about 50%, or by about 60%.

In some aspects, the peptides are modified to improve peptide stability. In some aspects, the modifying to improve peptide stability or the modifying to increase or reduce affinity to human proteases includes the substitution, deletion, or addition of one or more amino acid residues. In some aspects, the modifying improves peptide stability by about 20%, by about 30%, by about 40%, by about 50%, or by about 60%.

In some aspects, the peptides are modified to increase affinity to human proteases. In some aspects, the modifying increases affinity to human proteases by about 20%, by about 30%, by about 40%, by about 50%, or by about 60%. In some aspects, the peptides are modified to decrease affinity to human proteases. In some aspects, the modifying decreases affinity to human proteases by about 20%, by about 30%, by about 40%, by about 50%, or by about 60%.

In some aspects, selection of the peptide sequence from a regenerative protein sequence is based in part on the length or molecular weight of the peptide. In some aspects, the peptide sequence selected from the regenerative protein sequence is between about 5 to about 9 amino acids in length. In some aspects, the peptide sequence selected from the regenerative protein sequence is between about 10 to about 20 amino acids in length. In some aspects, the peptide sequence selected from the regenerative protein sequence is between about 21 to about 51 amino acids in length. In some aspects, the peptide sequence selected from the regenerative protein sequence is between about 51 to about 100 amino acids in length. In some aspects, the peptide sequence selected from the regenerative protein sequence is greater than 100 amino acids in length. In some aspects, the peptide sequence selected from the regenerative proteins sequence is between about 2 to about 4 amino acids in length.

In some aspects, the regenerative protein sequence from a regenerative species is one or more of the sequences of Table 1.

In some aspects, a peptide provided herein is a peptide of less than 55 amino acids in length, derived from a wild-type regenerative protein of Table 1, comprising at least one modification relative to the wild-type protein of Table 1, wherein the at least one modification improves one or more of human sequence homology, tissue penetration, peptide stability, and/or affinity to human proteases, and wherein the peptide regulates one or more of: myofibroblast formation, bacterial infection, inflammation, extracellular matrix production, keratin production, cell migration, or cell proliferation.

It is also contemplated that modified regenerative proteins (i.e., full length, or nearly full length protein sequences) derived from regenerative species may be useful as anti-fibrotic therapies. In some aspects, provided herein is a modified regenerative protein derived from a wild-type regenerative protein of a regenerative species, comprising at least one modification relative to the wild-type protein sequence, wherein the at least one modification improves one or more of human sequence homology, tissue penetration, peptide stability, and/or affinity to human proteases, and wherein the peptide regulates one or more of: myofibroblast formation, bacterial infection, inflammation, extracellular matrix production, keratin production, cell migration, or cell proliferation. In some aspects, the modified regenerative protein is derived from a wild-type regenerative protein of Table 1.

The peptides of the instant disclosure are tested for their efficacy with one or more assays. In some aspects, provided herein is a method of selecting one or more peptides from a subset of peptides for use in anti-fibrotic treatments comprising:
i) performing one or more assays on the peptides;
ii) comparing a result of the one or more assays of peptides relative to a control, wherein the one or more assays are selected from:
   a myofibroblast transition assay (MFT), a scratch assay, a proliferation assay, an inflammation assay, and an anti-microbial assay; and
iii) selecting the peptide for use in an anti-fibrotic treatment from the subset of peptides when the result of the one or more assays for the one or more given peptide is at least 40% greater than the control, or when the results of two or more assays for the one or more given peptides are both at least 25% greater than the control.

In some aspects, the one or more peptides are selected for use in anti-fibrotic treatments when:
i) the result of the MFT assay is at least 25%, at least 30%, at least 35%, at least 40%, at least 45%, at least 50%, at least 55%, at least 60%, at least 65%, at least 70%, at least 75%, or at least 80% greater than the control;
ii) the result of the scratch assay is at least 35%, at least 40%, at least 45%, or at least 50% greater than the control when performed on keratinocytes;
iii) the result of the scratch assay is at least 40%, at least 60%, at least 80%, at least 100%, at least 120%, at least 140%, at least 160%, or at least 180% greater than the control when performed on fibroblasts; or
iii) the result of the proliferation assay is at least 40%, at least 60%, at least 80%, or at least 100% greater than the control.

In some aspects, the one or more peptides are selected for use in anti-fibrotic treatments when the result of the inflammation assay is at least 20% greater than the control, or the result of the anti-microbial assay is at least 20% greater than the control.

In some aspects, the one or more peptides are selected for use in anti-fibrotic treatments when the peptide regulates collagen production, elastin production, hyaluronic acid production and/or keratin production as compared to a control in an in vitro assay.

In some aspects, the peptide selected for use in anti-fibrotic treatments comprises a sequence derived from a regenerative species regenerative protein sequence. In some aspects, the peptide selected comprises a sequence derived from an axolotl regenerative peptide sequence modified to increase cross-species homology.

In some aspects, the one or more peptides are selected for use in anti-fibrotic treatments when the result of one or more in vivo measurements is improved relative to the control. In some aspects, the one or more peptides are selected for use in anti-fibrotic treatments when the one or more in vivo measurements comprise a transepidermal water loss (TEWL) assay or erythema assay, wherein the result of the TEWL assay or erythema assay is at least 20%, at least 40%, at least 60%, at least 80%, at least 100%, at least 120%, at least 140%, or at least 160% greater than the control.

In some aspects, the one or more peptides are selected for use in anti-fibrotic treatments when the in vivo measurement shows improved healing time and/or degree of scarring relative to a control as measured by High-Resolution Computed Tomography (HRCT), Pulmonary Function Tests (PFTs), Bronchoscopy and Lung Biopsy, a Six-Minute Walk Test, and a Positron Emission Tomography (PET) Scan.

In some aspects, the one or more peptides are selected for use in anti-fibrotic treatments when the in vivo measurement shows improved healing time and/or degree of scarring relative to a control as measured by Kidney Biopsy and/or histopathology, Imaging Techniques (Ultrasound, Elastography, MRI), Blood and Urine Biomarkers (Serum Creatinine and Glomerular Filtration Rate, Proteinuria, Biomarkers, e.g., TGF-β), Functional Tests (Renal Scintigraphy, Clearance Studies) and Genetic and Molecular Tests (e.g., Transcriptomics and Proteomics).

In some aspects, the one or more peptides are selected for use in anti-fibrotic treatments when the in vivo measurement shows improved healing time and/or degree of scarring relative to a control as measured by Liver Biopsy, Non-Invasive Imaging Techniques (e.g., Transient Elastography (FibroScan), Magnetic Resonance Elastography (MRE), Acoustic Radiation Force Impulse (ARFI) Imaging, and Shear Wave Elastography (SWE)), Blood-Based Biomarkers and Serum Tests, Ultrasound, Computed Tomography (CT), Magnetic Resonance Imaging (MRI), Liver Function Tests (e.g., Serum Albumin, Bilirubin, and Prothrombin Time (PT)), and Genetic and Molecular Testing.

In some aspects, the one or more peptides are selected for use in anti-fibrotic treatments when the in vivo measurement shows improved healing time and/or degree of scarring relative to a control as measured by Clinical Examination (e.g., Visual Inspection, Palpation, and Functional Assessment). In some aspects, the thickness of the dermis, the density of collagen fibers, and the presence of inflammatory cells may be assessed to determine the efficacy of the peptide.

In some aspects, the one or more peptides are selected for use in anti-fibrotic treatments when the in vivo measurement shows improved healing time and/or degree of scarring relative to a control as measured by Imaging Techniques, e.g., High-Frequency Ultrasound, Optical Coherence Tomography (OCT), and Magnetic Resonance Imaging (MRI), Elastography (e.g., Cutaneous Elastography, Shear Wave Elastography), Skin Thickness Measurements, Patient-Reported Outcome Measures (PROMs), Advanced Imaging Techniques (e.g., Confocal Microscopy), and/or Molecular Techniques (e.g., Genetic or Molecular Testing).

In some aspects, the one or more peptides are selected for use in anti-fibrotic treatments when the in vivo measurement shows improved healing time and/or degree of scarring. In some aspects, the one or more peptides are selected for use in anti-fibrotic treatments when the in vivo measurement shows improvement relative to a control after a scrape biopsy, full biopsy, punch biopsy, chemical peel, laser peel, dermabrasion, micro-needling, tape stripping, burn of the skin, or surgery. In some aspects the in vivo measurement is performed after inducing contact dermatitis. In some aspects the in vivo measurement is performed on psoriasis plaques.

In some aspects, the one or more peptides are selected for use in anti-fibrotic treatments when the in vivo measurement shows improvement relative to a control of one or more of collagen production, elastin production, hyaluronic acid production, keratin production, firmness of the skin, elasticity of the skin, smoothness of the skin, appearance of wrinkles, depth of wrinkles, hyperpigmentation, acne scarring, skin tone evenness, melasma, skin cell turnover, stretch marks, cellulite, skin surface hydration, or skin brightness.

In some aspects, provided herein is a method of selecting a peptide from a subset of peptides for use in anti-fibrotic treatments comprising: i) performing an RNA-seq assay on the peptide; ii) comparing the result of the assay on the peptide relative to a control; and iii) selecting the peptide for use in anti-fibrotic treatments from the subset of peptides when the result of the RNA-seq assay is improved relative to the control.

In some aspects, provided herein is a method of selecting a peptide from a subset of peptides for use in anti-fibrotic treatments comprising: i) performing a DNA damage assay on the peptide; ii) comparing the result of the assay on the peptide relative to a control; and iii) selecting the peptide for use in anti-fibrotic treatments from the subset of peptides when the result of the DNA damage assay is improved relative to the control.

In some aspects, the peptides provided herein include, but are not limited to, peptides comprising an amino acid sequence of Table 2 or an amino acid sequence with at least 70% sequence identity thereto, wherein the peptide comprises at least one modification relative to a wild-type sequence.

In some aspects, the amino acid sequence of a peptide provided herein comprises a sequence of P2, or a sequence with at least 70% sequence identity thereto, with at least one modification relative to the wild-type sequence of Epiplakin; or a sequence of P3, or a sequence with at least 70% sequence identity thereto, with at least one modification relative to the wild-type sequence of FGF-binding protein.

In some aspects, the peptide sequence comprising an amino acid sequence of Table 2 is derived from a regenerative species regenerative protein and is modified to increase cross-species sequence homology.

In some aspects, provided herein are compositions comprising one or more peptides, and one or more adjuvants or excipients. In some aspects, the composition is for use in anti-fibrotic treatments. In some aspects, the anti-fibrotic treatment comprises scar prevention or reduction, post-surgical wound healing, tissue or organ generation, chronic wound healing, wound healing associated with diseases or disorders, e.g., epithelial disorders, age-related skin impact, treatment of burns, reduction of inflammation, reduction of infection, or combinations thereof.

In some aspects, the anti-fibrotic treatment treats fibrosis associated with or caused by a disease or disorder that produces fibrotic tissue throughout one or more organs. In some aspects the fibrosis is associated with or caused by cancer or a metabolic disorder. In some aspects the fibrosis is associated with or caused by a genetic disorder. In some aspects the fibrosis is associated with or caused by surgery, injury, infection, or substance use, e.g., alcohol or smoking. In some aspects the fibrosis is associated with or caused by exposure to allergens.

In some aspects, the anti-fibrotic treatment treats liver fibrosis. In some aspects the liver fibrosis is hepatic fibrosis or cirrhosis. In some aspects the hepatic fibrosis is due to chronic hepatitis or abuse of alcohol. In some aspects the fibrosis is associated with or caused by liver cancer.

In some aspects, the anti-fibrotic treatment treats lung fibrosis. In some aspects the lung fibrosis is Idiopathic Pulmonary Fibrosis (IPF). In some aspects the lung fibrosis is associated or caused by Pneumoconiosis. In some aspects the lung fibrosis is associated with or caused by inhalation of dust, asbestosis or silicosis. In some aspects the lung fibrosis is associated with or caused by Chronic Hypersensitivity Pneumonitis. In some aspects the lung fibrosis is associated or caused by exposure to allergens. In some aspects the lung fibrosis is associated with or caused by Cystic Fibrosis. In some aspects the fibrosis is associated with or caused by lung cancer.

In some aspects, the anti-fibrotic treatment treats cardiac fibrosis. In some aspects the cardiac fibrosis is caused by or associated with a heart condition, e.g., a myocardial infarction and/or heart failure. In some aspects the cardiac fibrosis is associated with or caused by Hypertrophic Cardiomyopathy. In some aspects, the cardiac fibrosis is associated with or caused by a genetic disorder.

In some aspects, the anti-fibrotic treatment treats kidney fibrosis. In some aspects the kidney fibrosis is associated with or caused by Chronic Kidney Disease (CKD). In some aspects the kidney fibrosis includes interstitial fibrosis. In some aspects the kidney fibrosis is Diabetic Nephropathy. In some aspects the kidney fibrosis is associated with or caused by chronic diabetes.

In some aspects, the anti-fibrotic treatment treats pancreatic fibrosis. In some aspects, the fibrosis is associated with or caused by Chronic Pancreatitis. In some aspects the pancreatic fibrosis is associated with or caused by Cystic Fibrosis. In some aspects the fibrosis is associated with or caused by pancreatic cancer.

In some aspects, the anti-fibrotic treatment treats intestinal fibrosis. In some aspects, the intestinal fibrosis is associated with or caused by Crohn's Disease. In some aspects, the fibrosis is associated with or caused by a bowel disease. In some aspects, the intestinal fibrosis is associated with or caused by Radiation Enteritis.

In some aspects, the anti-fibrotic treatment treats ocular fibrosis. In some aspects, the fibrosis is associated with or caused by Proliferative Vitreoretinopathy. In some aspects the ocular fibrosis is associated with or caused by ocular surgery. In some aspects the ocular fibrosis is associated with or caused by Corneal Fibrosis. In some aspects the ocular fibrosis is associated with or caused by injury or infection.

In some aspects, the anti-fibrotic treatment treats bone marrow fibrosis. In some aspects, the fibrosis is associated with or caused by Myelofibrosis. In some aspects, the fibrosis is associated with or caused by a blood cell abnormality or blood cancer.

In some aspects, the anti-fibrotic treatment treats uterine fibrosis. In some aspects, the fibrosis is associated with or caused by Asherman's Syndrome. In some aspects the fibrosis is associated with or caused by endometriosis. In some aspect the fibrosis is ovarian fibrosis. In some aspects, the fibrosis is associated with or caused by uterine or ovarian cancer.

In some aspects, the anti-fibrotic treatment comprises the prevention or reduction of scar formation. In some aspects, the prevention or reduction of scar formation is post-surgical. In some aspects, the anti-fibrotic treatment comprises generating or regenerating an organ or tissue. In some aspects, the anti-fibrotic treatment improves pressure ulcers. In some aspects, the anti-fibrotic treatment heals burns. In some aspects, the anti-fibrotic treatment heals wounds associated with diseases or disorders, e.g., epithelial skin disorders. In some aspects, the anti-fibrotic treatment heals wounds associated with diabetes, e.g., diabetic ulcers, e.g., of the foot. In some aspects, the anti-fibrotic treatment heals psoriasis. In some aspects, the anti-fibrotic treatment heals dermatitis. In some aspects, the anti-fibrotic treatment heals rosacea. In some aspects, the anti-fibrotic treatment heals acne. In some aspects, the anti-fibrotic treatment heals diaper rash.

In some aspects, the anti-fibrotic treatment reduces inflammation. In some aspects, the anti-fibrotic treatment reduces inflammation associated with a dermatological treatment. In some aspects, the dermatological treatment is laser, micro-needling, or dermabrasion.

In some aspects, the anti-fibrotic treatment reduces aging-related skin impact. In some aspects the anti-fibrotic treatment reduces or ameliorates aging, or improves the health or appearance of the skin.

In some aspects, the composition is formulated together with a skin protectant, wound healing, or acne treatment monograph to produce a peptide formulation. In some aspects, the formulation comprises a skin penetration enhancer. In some aspects, the formulation is applied topically as a serum, gel, lotion, cream, ointment, mask, peel, or spray.

In some aspects, the peptide formulation is applied with a silicon or allantoin gel. In some aspects, the formulation is applied with a silicon patch. In some aspects, the formulation comprises one or more of salicylic acid, benzoyl peroxide, resorcinol, resorcinol monoacetate, or sulfur.

Also provided herein are methods of treatment with the regenerative peptides. In some aspects, provided herein is a method of treatment comprising:

topically applying a formulation comprising a peptide to an area to be treated, wherein the peptide is derived from a regenerative species regenerative protein, and wherein the peptide is modified to include one or more amino acid residues from the homologous human sequence, wherein the peptide regulates one or more of: myofibroblast transition, bacterial infection, inflammation, cell migration, or cell proliferation.

In some aspects, the method of treatment reduces fibrosis. In some aspects, the treatment comprises a reduction of fibrosis for tissue or organ generation, chronic wound healing, or fibrosis associated with diseases or disorders.

In some aspects, the anti-fibrotic treatment treats systemic scleroderma, localized scleroderma (i.e., morphea), eosinophilic fasciitis (i.e., Shulman's syndrome), lipodermatosclerosis, nephrogenic systemic fibrosis (NSF), scleredema, scleromyxedema, diffuse cutaneous systemic sclerosis, chronic graft-versus-host Disease (cGVHD) with skin involvement, dermatofibrosarcoma protuberans (DFSP), sclerotic chronic graft-versus-host disease (cGVHD), lichen sclerosus, calciphylaxis (calcific uremic arteriolopathy), acrodermatitis chronica atrophicans, poikiloderma of civatte, epidermolysis bullosa, lichen planus, keloids, hypertrophic scarring, and necrobiosis lipoidica.

In some aspects, the anti-fibrotic treatment treats epidermolysis bullosa, localized scleroderma, or necrobiosis lipoidica.

In some aspects the anti-fibrotic treatment treats keloids, hypertrophic scarring, systemic scleroderma, lichen planus, lichen sclerosus, or lipodermatosclerosis. In some aspects, the reduction of fibrosis reduces the inflammation, thickening, and/or hardening of a tissue.

In some aspects, the anti-fibrotic treatment is a treatment used in organ or tissue regeneration, wound healing, treatment of epithelial disorders, treatment of age-related skin impact, treatment of burns, reduction of inflammation, reduction of infection, or combinations thereof. In some aspects, the anti-fibrotic treatment comprises the prevention or reduction of scar formation. In some aspects, the scar formation is post-surgical.

In some aspects, the anti-fibrotic treatment treats fibrosis associated with or caused by a disease or disorder. In some aspects, the disease is cancer. In some aspects, the disease or disorder is a metabolic disorder. In some aspects, the disorder is a genetic disorder. In some aspects, the anti-fibrotic treatment treats fibrosis associated with or caused by surgery, injury, infection, or substance use. In some aspects, the anti-fibrotic treatment treats fibrosis associated with or caused by exposure to allergens.

In some aspects, the anti-fibrotic treatment treats liver fibrosis. In some aspects, the liver fibrosis is hepatic fibrosis or cirrhosis. In some aspects, the anti-fibrotic treatment treats lung fibrosis. In some aspects, the lung fibrosis is Idiopathic Pulmonary Fibrosis (IPF), Pneumoconiosis, Chronic Hypersensitivity Pneumonitis, or Cystic Fibrosis. In some aspects, the anti-fibrotic treatment treats cardiac fibrosis. In some aspects, cardiac fibrosis is caused by or associated with a heart condition. In some aspects, the anti-fibrotic treatment treats kidney fibrosis. In some aspects, the kidney fibrosis is associated with or caused by Chronic Kidney Disease (CKD) or Diabetic Nephropathy.

In some aspects, the anti-fibrotic treatment treats pancreatic fibrosis. In some aspects, the pancreatic fibrosis is associated with or caused by Chronic Pancreatitis or Cystic Fibrosis. In some aspects, the anti-fibrotic treatment treats intestinal fibrosis. In some aspects, the intestinal fibrosis is associated with or caused by Crohn's Disease or Radiation Enteritis. In some aspects, the anti-fibrotic treatment treats ocular fibrosis. In some aspects, the ocular fibrosis is associated with or caused by Proliferative Vitreoretinopathy or Corneal Fibrosis. In some aspects, the anti-fibrotic treatment treats bone marrow fibrosis. In some aspects, the fibrosis is Myelofibrosis. In some aspects, the anti-fibrotic treatment treats uterine fibrosis. In some aspects, the fibrosis is associated with or caused by Asherman's Syndrome.

In some aspects, the anti-fibrotic treatment improves pressure ulcers. In some aspects, the treatment is applied to burns. In some aspects, the anti-fibrotic treatment treats epithelial skin disorders. In some aspects, the anti-fibrotic treatment treats psoriasis. In some aspects, the anti-fibrotic treatment treats dermatitis. In some aspects, the anti-fibrotic treatment treats rosacea. In some aspects, the anti-fibrotic treatment treats acne. In some aspects, the anti-fibrotic treatment reduces inflammation.

In some aspects, the inflammation is associated with a dermatological treatment. In some aspects, the dermatological treatment is laser, micro-needling, or dermabrasion.

In some aspects, the anti-fibrotic treatment reduces age-related skin impact. In some aspects the anti-fibrotic treatment reduces or ameliorates aging, or improves the health or appearance of the skin.

In some aspects, the anti-fibrotic peptide is formulated together with a skin protectant, wound healing, or acne treatment monograph to produce a peptide formulation. In some aspects, the peptide formulation further comprises a skin penetration enhancer.

In some aspects, the peptide formulation is applied topically as a serum, gel, lotion, cream, ointment, mask, peel, or spray. In some aspects, the peptide formulation is applied with a silicon or allantoin gel. In some aspects, the peptide formulation is applied with a silicon patch.

In some aspects, the formulation comprises one or more of salicylic acid, benzoyl peroxide, resorcinol, resorcinol monoacetate, or sulfur.

In some aspects, the anti-fibrotic peptide is a peptide selected from Table 2.

In some aspects, provided herein are methods of reducing fibrosis comprising applying a formulation comprising a peptide made by the method comprising:
i) selecting a peptide sequence from a regenerative protein sequence from a regenerative species; and
ii) modifying the peptide sequence to improve one or more of cross-species homology, tissue penetration, peptide stability, and/or affinity to human proteases; and
iii) synthesizing the peptide;
wherein the peptide regulates one or more of: myofibroblast formation, bacterial infection, inflammation, cell migration, extracellular matrix production, keratin production, or cell proliferation. In some aspects, the wound healing protein sequence from a regenerative species is one or more of the sequences of Table 1.

Also contemplated herein is a method of synthesizing a peptide for wound healing comprising:
i) selecting a peptide sequence from a wound healing protein sequence from a regenerative species; and
ii) modifying the peptide sequence to improve one or more of cross-species homology, tissue penetration, peptide stability, and/or affinity to human proteases; and iii) synthesizing the peptide; wherein the peptide regulates one or more of: fibrosis, bacterial infection, inflammation, cell migration, extracellular matrix production, keratin production or cell proliferation. It is also contemplated that modified wound healing proteins (i.e., full length, or nearly full length protein sequences) derived from regenerative species may be useful as wound healing therapies. In some aspects, provide herein is a modified wound healing protein derived from a wild type wound healing protein of a regenerative species, comprising at least one modification relative to the wild type protein sequence, wherein the at least one modification improves one or more of human sequence homology, tissue penetration, peptide stability, and/or affinity to human proteases, and wherein the peptide regulates one or more of: fibrosis, bacterial infection, inflammation, extracellular matrix production, keratin production, cell migration, or cell proliferation.

In some aspects, provided herein is a method of selecting one or more peptides from a subset of peptides for use in wound healing comprising:
i) performing one or more assays on the peptides;
ii) comparing a result of the one or more assays of peptides relative to a control, wherein the one or more assays are selected from: a myofibroblast transition assay (MFT), a scratch assay, a proliferation assay, an inflammation assay, and an anti-microbial assay; and
iii) selecting the peptide for use in wound healing from the subset of peptides when the result of the one or more assays for the one or more given peptide is at least 40% greater than the control, or when the results of two or more assays for the one or more given peptides are both at least 25% greater than the control. Also provided herein are methods of treatment with a peptide for use in wound healing. In some aspects, the wound healing treatment comprises tissue or organ generation, reduction of fibrosis, chronic wound healing, wound healing associated with diseases or disorders, e.g., epithelial disorders, age-related skin impact, treatment of burns, reduction of inflammation, reduction of infection, or combinations thereof.

### BRIEF DESCRIPTION OF THE DRAWINGS

FIG. 1 is a diagram representing gene expression, from left to right, of human adults and embryos, a hypothetical ideal human, and the regenerative species of axolotl, zebrafish, and spiny mouse in response to tissue damage from inflammation, wounds, disease, or infection.

FIG. 2 is an annotated graph showing the effect of the exemplary P2 peptide on fibroblast scratch assay recovery after 48 hours.

FIG. 3 is an annotated graph showing the effect of the exemplary P3 peptide on fibroblast to myoblast transition (MFT), as indicated by the levels of alpha-smooth muscle actin (aSMA).

FIG. 4 is a control Table showing the agar zone of inhibition with the antibiotics Chloramphenicol and Tetracycline with the bacterial strains Staphylococcus aureus USA300 (NRS384), Staphylococcus aureus ST80, Staphylococcus aureus JE2, Staphylococcus aureus MRSA col, Pseudomonas aeruginosa ATCC 27853, and Acinetobacter baumannii ATCC 196062.

FIG. 5 is a Table showing the agar zone of inhibition of the MBb32 peptide with the same bacterial strains tested in FIG. 4 and at concentrations ranging from 62.5 μg/ml to 1000 μg/ml.

FIG. 6 is MIC50 fit graphs of the MBb32 peptide as a function of growth inhibition of bacteria in broth.

FIG. 7 is a Table summarizing the calculated MIC50 values for the MBb32 peptide as a function of the growth inhibition of bacteria in broth shown in FIG. 7.

FIG. 8 is a Table summarizing the calculated MIC90 values for the MBb32 peptide as a function of the growth inhibition of bacteria in broth shown in FIG. 7.

FIG. 9 is a Table showing the minimum bactericidal concentration (MBC) at which the MBb32 peptide will kill each bacterial strain on agar plates.

FIG. 10 is a Table showing the agar zone of inhibition of the MBdol15, and MBdol20 MBdol23 peptides in Streptococcus pneumoniae DCC1335 and Acinetobacter baumannii ATCC 196062 at concentrations ranging from 62.5 μg/ml to 1000 μg/ml.

FIG. 11 is a Table summarizing the calculated MIC50 and MIC90 values for the MBdol15, and MBdol20, and MBdol23 peptides, as a function of the growth inhibition of bacteria in broth, in Staphylococcus aureus USA300 (NRS384), Staphylococcus aureus ST80, Staphylococcus aureus JE2, Klebsiella pneumoniae ATCC 10031, Pseudomonas aeruginosa ATCC 27853, and Acinetobacter baumannii ATCC 196062.

FIG. 12 is a Table showing the minimum bactericidal concentration (MBC) at which the MBdol15 and MBdol20 peptides will kill each bacterial strain on agar plates.

FIG. 13 is a graph showing the effects of the exemplary peptides P2 and P3 on transepidermal water loss (TEWL) at 8 hours, 24 hours, and 48 hours after dermabrasion.

FIG. 14 is a graph showing the effects of the exemplary peptides P2 and P3 on erythema (skin redness) at 4 hours and 12 hours after dermabrasion.

FIGS. 15A and 15B are Visioscan images of skin smoothness showing the effects of exemplary peptides P2 and P3 at 8 hours, 24 hours, 48 hours, and 72 hours after dermabrasion. FIG. 15A shows P2 effects after dermabrasion; FIG. 15B shows P3 effects after dermabrasion.

FIG. 16 is a Table showing an exemplary vehicle cream, the ingredients and methods of producing same, as used in the clinical studies of FIGS. 13, 14, 15A, and 15B.

FIG. 17 is images of an example alpha-smooth muscle actin (aSMA) immunofluorescence stain in control (left) human dermal fibroblasts and in those treated with 433 μM MBb11 (right).

FIGS. 18A and 18B show the effects of MBb11 on alpha-smooth muscle actin (aSMA) expression. FIG. 18A is a graph of aSMA expression levels measured by immunofluorescence staining as a function of MBb11 concentration. FIG. 18B is an image of aSMA expression as measured by Western blot of the aSMA protein.

FIG. 19 is a graph of cell viability of human dermal fibroblasts in response to MBb11 as a function of MBb11 concentration.

FIG. 20 is images of an example alpha-smooth muscle actin (aSMA) immunofluorescence stain in control (left) human dermal fibroblasts and in those treated with 412 μM MBb32 (right).

FIG. 21 is a graph of aSMA expression levels measured by immunofluorescence staining in response to MBb32 treatment as a function of MBb32 concentration

FIG. 22 is a graph of cell viability of human dermal fibroblasts in response to MBb32 as a function of MBb32 concentration.

FIG. 23 is images of an example alpha-smooth muscle actin (aSMA) immunofluorescence stain in human lung fibroblasts treated with 50 μM MBb32 (right) and 200 μM MBb32.

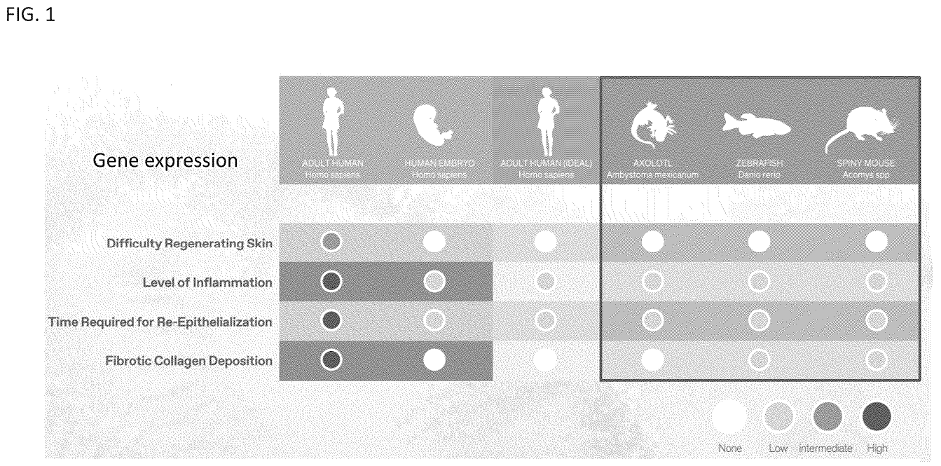

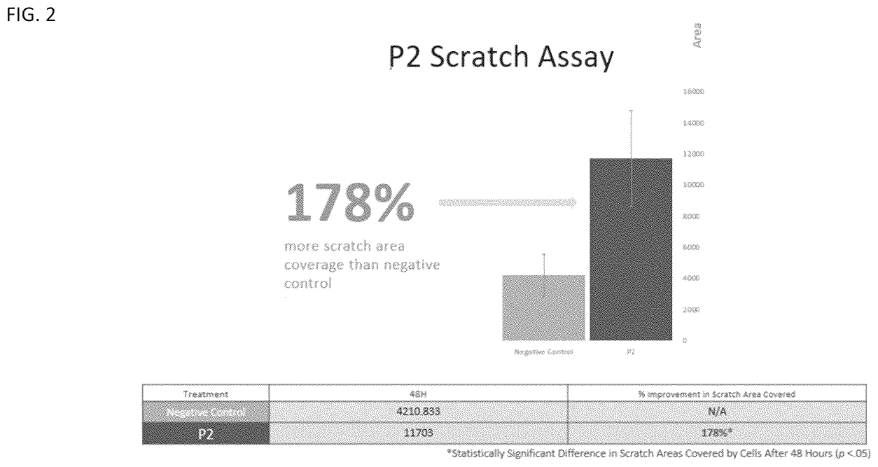

### DETAILED DESCRIPTION

Provided herein are peptide compositions derived from regenerative species proteins, and methods of making and use thereof, that seek to reduce fibrosis as anti-fibrotic treatments. In most mammals, replacement of tissue in response to inflammation, wounds, disease, or infection results in a thickening or hardening of the tissue known as fibrosis. However, regenerative species and human embryos rely on alternative pathways that result in “complete” tissue regeneration, in which no fibrosis is detected. Provided herein are peptide compositions derived from regenerative species regenerative proteins, e.g., regenerative proteins from an Axolotl salamander (Ambystoma mexicanum), and methods of making and use thereof, that seek to reduce fibrosis in humans. The peptides provided herein derived from regenerative species proteins may also be used as wound healing treatments.

The peptides of the instant disclosure demonstrated efficacy in a number of assays that support their utility as anti-fibrotic treatments. For example, the peptides improve in vitro measures including a reduction of fibroblast to myofibroblast transition (MFT), increased cell proliferation, and increased scratch assay wound closure. The peptides also reduced bacterial growth and inflammation markers in vitro and improve clinical in vivo measures of a reduction in fibrosis including skin smoothness, skin permeability, and skin redness post dermabrasion

The peptides of the instant disclosure may also be used as wound healing treatments. In most mammals, epithelial wound healing is incomplete in that the replacement tissue produced by the body, i.e., the scar tissue, differs from the native tissue in both appearance and functionality. These differences in replacement tissue are due to the inflammatory response induced by the wound, any infection present, and the differential organization of fibrotic collagen deposited by myofibroblasts during the wound healing response. However, regenerative species and human embryos rely on alternative pathways that result in “complete” wound healing, in which the replacement tissue is indistinguishable from the native tissue. Provided herein are peptide compositions derived from regenerative species proteins, and methods of making and use thereof, that seek to improve wound healing in humans. Improvements to wound healing may include scar prevention or reduction, as well as restoration of key structural compounds to the skin, e.g., collagen, elastin, hyaluronic acid, and keratin to improve skin integrity, restoration of skin barrier function, and/or reduction of the inflammation and pain associated with a wound.

The peptides of the instant disclosure demonstrated efficacy in a number of assays that support their utility as wound healing treatments: for example, the peptides improve in vitro measures of wound healing including fibroblast to myofibroblast transition (MFT), cell proliferation, and scratch assay wound closure; the peptides also reduce bacterial growth and inflammation markers in vitro; and the peptides improve clinical in vivo measures of wound healing including skin smoothness, skin permeability, and skin redness post dermabrasion.

### I. Fibrosis

The peptide compositions of the instant disclosure are derived from regenerative species proteins and are intended to reduce fibrosis as anti-fibrotic treatments.

Fibrosis is a thickening or hardening of tissue and is caused by myofibroblast collagen and fibronectin deposited in tissue in response to perturbations including but not limited to inflammation, wounds, disease, or infection. Anti-fibrotic treatments may include reduction of scar formation; restoring key structural compounds to the tissue, e.g., collagen, elastin, hyaluronic acid, and keratin; restoring epithelial barrier function; reducing the pain of the fibrosis; reducing the disease causing the fibrosis; and reducing the underlying inflammation, wounds, or infection causing the fibrosis.

In some aspects the fibrosis results from or is associated with systemic scleroderma, localized scleroderma (i.e., morphea), eosinophilic fasciitis (i.e., Shulman's syndrome), lipodermatosclerosis, nephrogenic systemic fibrosis (NSF), scleredema, scleromyxedema, diffuse cutaneous systemic sclerosis, chronic graft-versus-host Disease (cGVHD) with skin involvement, dermatofibrosarcoma protuberans (DFSP), sclerotic chronic graft-versus-host disease (cGVHD), lichen sclerosus, calciphylaxis (calcific uremic arteriolopathy), acrodermatitis chronica atrophicans, poikiloderma of civatte, epidermolysis bullosa, lichen planus, keloids, hypertrophic scarring, or necrobiosis lipoidica.

In some aspects the fibrosis results from or is associated with epidermolysis bullosa, localized scleroderma, or necrobiosis lipoidica.

In some aspects the fibrosis is associated with keloids, hypertrophic scarring, systemic scleroderma, lichen planus, lichen sclerosus, or lipodermatosclerosis.

In some aspects, the fibrosis is associated with or caused by a disease or disorder that produces fibrotic tissue throughout one or more organs. In some aspects the fibrosis is associated with or caused by cancer or a metabolic disorder. In some aspects the fibrosis is associated with or caused by a genetic disorder. In some aspects the fibrosis is associated with or caused by surgery, injury, infection, or substance use, e.g., alcohol or smoking. In some aspects the fibrosis is associated with or caused by exposure to allergens.

In some aspects the fibrosis is fibrosis of the liver. In some aspects the liver fibrosis is hepatic fibrosis or cirrhosis. In some aspects the hepatic fibrosis is due to chronic hepatitis or abuse of alcohol. In some aspects the fibrosis is associated with or caused by liver cancer.

In some aspects the fibrosis is fibrosis of the lung. In some aspects the lung fibrosis is Idiopathic Pulmonary Fibrosis (IPF). In some aspects the lung fibrosis is associated or caused by Pneumoconiosis. In some aspects the lung fibrosis is associated with or caused by inhalation of dust, asbestosis or silicosis. In some aspects the lung fibrosis is associated with or caused by Chronic Hypersensitivity Pneumonitis. In some aspects the lung fibrosis is associated or caused by exposure to allergens. In some aspects the lung fibrosis is associated with or caused by Cystic Fibrosis. In some aspects the fibrosis is associated with or caused by lung cancer.

In some aspects the fibrosis is cardiac fibrosis. In some aspects the cardiac fibrosis is caused by or associated with a heart condition, e.g., a myocardial infarction and/or heart failure. In some aspects the cardiac fibrosis is associated with or caused by Hypertrophic Cardiomyopathy. In some aspects, the cardiac fibrosis is associated with or caused by a genetic disorder.

In some aspects the fibrosis is kidney fibrosis. In some aspects the kidney fibrosis is associated with or caused by Chronic Kidney Disease (CKD). In some aspects the kidney fibrosis includes interstitial fibrosis. In some aspects the kidney fibrosis is Diabetic Nephropathy. In some aspects the kidney fibrosis is associated with or caused by chronic diabetes.

In some aspects the fibrosis is pancreatic fibrosis. In some aspects, the fibrosis is associated with or caused by Chronic Pancreatitis. In some aspects the pancreatic fibrosis is associated with or caused by Cystic Fibrosis. In some aspects the fibrosis is associated with or caused by pancreatic cancer.

In some aspects the fibrosis is intestinal fibrosis. In some aspects, the intestinal fibrosis is associated with or caused by Crohn's Disease. In some aspects, the fibrosis is associated with or caused by a bowel disease. In some aspects, the intestinal fibrosis is associated with or caused by Radiation Enteritis.

In some aspects the fibrosis is ocular fibrosis. In some aspects, the fibrosis is associated with or caused by Proliferative Vitreoretinopathy. In some aspects the ocular fibrosis is associated with or caused by ocular surgery. In some aspects the ocular fibrosis is associated with or caused by Corneal Fibrosis. In some aspects the ocular fibrosis is associated with or caused by injury or infection.

In some aspects the fibrosis is bone marrow fibrosis. In some aspects, the fibrosis is associated with or caused by Myelofibrosis. In some aspects, the fibrosis is associated with or caused by a blood cell abnormality or blood cancer.

In some aspects the fibrosis is uterine fibrosis. In some aspects, the fibrosis is associated with or caused by Asherman's Syndrome. In some aspects the fibrosis is associated with or caused by endometriosis. In some aspect the fibrosis is ovarian fibrosis. In some aspects, the fibrosis is associated with or caused by uterine or ovarian cancer.

In some aspects, the fibrosis is associated with tissue or organ generation, chronic wound healing, and wound healing associated with diseases or disorders.

In some aspects, the fibrosis is associated with wound healing. A wound as provided herein refers to any tear, damage, or disruption to the skin or internal tissue. A wound may include both micro-wounds, e.g., on the order of a microscopic tear in the skin, macro wounds, e.g., visible disruption to entire tissues, or any size in between. As provided herein, a wound refers to any acute or accumulated epithelial tissue impact. Wound healing as used herein, refers to the biological processes employed by an animal to replace wounded cells or tissue.

In some aspects, the fibrosis is associated with post-surgical wounds, a wound associated with a disease, e.g. organ inflammation and fibrosis, a pressure ulcer, or a burn. In some aspects, organ generation or regeneration therapies may benefit from an anti-fibrotic treatment as provided herein.

In some aspects, the fibrosis is associated with an epithelial disorder such as psoriasis, dermatitis, rosacea, or acne. In some aspects, the fibrosis is associated with a dermatological treatment, e.g., laser, micro-needling, or dermabrasion.

In some aspects, the fibrosis is associated with the accumulation of micro-wounds that are associated with aging. In some aspects, an anti-fibrotic treatment treats the accumulation of micro-wounds associate with internal or external factors, e.g., UV light exposure or smoking. In some aspects, an anti-fibrotic treatment treats differential collagen production or organization associated with aging, the presence and/or deepening of wrinkles, hyperpigmentation, decreased skin cell turnover, acne scarring, loss of firmness, decreased elasticity, decreased smoothness, melasma, stretch marks, cellulite, decreased skin surface hydration, and/or decreased skin brightness.

### II. Wounds and Wound Healing

The peptide compositions of the instant disclosure derived from regenerative species proteins may also be used to improve wound healing. Improvements to wound healing may include scar reduction, restoring key structural compounds to the skin, e.g., collagen, elastin, hyaluronic acid, and keratin, restoring skin barrier function, and reducing the inflammation and/or pain associated with a wound. A “wound” as provided herein refers to any tear, damage, or disruption to the skin or internal tissue. A wound may include both micro-wounds, e.g., on the order of a microscopic tear in the skin, macro wounds, e.g., visible disruption to entire tissues, or any size in between. As provided herein, a wound refers to any acute or accumulated epithelial tissue impact, including but not limited to those resulting from physical trauma, infection, burns, inflammation, surgery, or disease. “Wound healing,” as used herein, refers to the biological processes employed by an animal to replace wounded cells or tissue, as well the biological processes employed to control inflammation and infection associated with the wound. A wound healing protein thus refers to a protein regulating, for example, any of fibrosis, bacterial infection, inflammation, extracellular matrix or keratin production, cell migration, and/or cell proliferation.

As contemplated herein, a wound is associated with scar formation, including but not limited to post-surgical wounds, a wound associated with a disease, e.g. organ inflammation and fibrosis, a pressure ulcer, or a burn. In some aspects, organ generation or re-generation therapies may benefit from a wound healing treatment as provided herein. In some aspects, a wound refers to the condition of skin with an epithelial disorder such as psoriasis, dermatitis, rosacea, or acne. In some aspects, a wound may be associated with a dermatological treatment, e.g., laser, micro-needling, or dermabrasion.

In some aspects, a wound refers to the accumulation of micro-wounds that are associated with aging. In some aspects, other internal or external factors, e.g., UV light exposure or smoking, are associated with the accumulation of micro-wounds. In some aspects, micro-wound accumulation is associated with differential collagen production or organization, the presence and/or deepening of wrinkles, hyperpigmentation, decreased skin cell turnover, acne scarring, loss of firmness, decreased elasticity, decreased smoothness, melasma, stretch marks, cellulite, decreased skin surface hydration, and/or decreased skin brightness.

### III. Regenerative Species Regenerative Proteins

The peptides of the instant disclosure are at least in part, or initially, derived from regenerative proteins of regenerative species. Regenerative species refers to any adult animal with the ability to heal from inflammation, wounds, disease, or infection with tissue that is essentially indistinguishable from that of the native tissue. Examples of regenerative species include axolotl, zebrafish, spiny mouse, Xenopus, Mexican tetra, hydra, newt, starfish, and planarian among others. Animals may regenerate to varying degrees, with some animals able to regenerate limbs and other body parts identical to the native tissues. To accomplish the replacement of tissue in regenerative species, differential gene regulation during healing may include, for example, genes involved in cell proliferation, extracellular matrix or keratin production, cell migration, inflammation, and response to infection.

Information regarding gene expression of regenerative species regenerative proteins may be considered in helping to determine an initial peptide source. Sources for gene expression information during tissue regeneration in regenerative species may include, for example, single-cell RNA sequencing, bulk RNA sequencing, epigenetic regulation, and translatomic information. The regenerative proteins used as a source for the peptides of the instant disclosure may also be selected, in part, based on their ability to function as a ligand, the relevance of the signaling pathway, the size of the protein, and whether the ligand functions intracellularly or extracellularly.

In some exemplary aspects, the regenerative protein used as a source for the peptides is an axolotl protein expressed during tissue regeneration, and optionally involved in one or more of infection, inflammation, cell migration, or cell proliferation. Some exemplary proteins expressed in axolotl during tissue regeneration, and that were used as a source for the peptides of the instant disclosure include those of Table 1 below. In some aspects, a regenerative species regenerative protein may also be derived from another regenerative species.

TABLE 1
Exemplary Regenerative Species Regenerative Proteins
Protein
Arf-GAP with Rho-GAP domain, ANK repeat and PH
domain-containing protein 1
A disintegrin and metalloproteinase with thrombospondin motifs 17
Reticulocalbin-1
Rho GTPase-activating protein 22
Tomoregulin-1
Tyrosine-protein kinase transmembrane receptor ROR2
Scaffold attachment factor B1
Collagen alpha-1 (III) chain
Far upstream element-binding protein 2
DNA excision repair protein ERCC-6-like
Cell division cycle-associated 7-like protein
Glutaredoxin-like protein C5orf63
Rho GTPase-activating protein 28
Myosin 8
Collagen alpha-1(XI) chain
E3 ubiquitin-protein ligase TRIM71
25-hydroxyvitamin D-1 alpha hydroxylase, mitochondrial
Polypyrimidine tract-binding protein 1
Protein SSUH2 homolog
LIM domain and actin-binding protein 1
UDP-glucose: glycoprotein glucosyltransferase 1
UDP-glucose: glycoprotein glucosyltransferase 7
Matrix metalloproteinase-2
FERM, ARHGEF and pleckstrin domain-containing protein 1
Matrix metalloproteinase-14
A disintegrin and metalloproteinase with thrombospondin motifs 18
Cytochrome P450 2C8
Unconventional myosin-Ie
Phosphatidylinositol 3-kinase regulatory subunit gamma
25-hydroxyvitamin D-1 alpha hydroxylase
Retinol dehydrogenase 11
RNA-binding protein 45
Serine/threonine-protein kinase Chk1
Mitogen-activated protein kinase 11
Dual specificity protein phosphatase 6
Double-stranded RNA-specific adenosine deaminase
DNA (cytosine-5)-methyltransferase 3B
Retinol dehydrogenase 12
Adhesion G protein-coupled receptor L2
Heterogeneous nuclear ribonucleoprotein R
Tyrosine-protein kinase transmembrane receptor ROR3
Heterogeneous nuclear ribonucleoprotein M
WD repeat-containing protein 43
Matrix metalloproteinase-3
FERM, ARHGEF and pleckstrin domain-containing protein 2
Septin-9
Glycosyltransferase 8 domain-containing protein 2
Heterogeneous nuclear ribonucleoprotein L
Carbonyl reductase [NADPH] 1
Heterogeneous nuclear ribonucleoprotein Q
Far upstream element-binding protein 3
Dixin
Peroxiredoxin-4
E3 ubiquitin-protein ligase TRIM72
tRNA (uracil-5-)-methyltransferase homolog A
UDP-glucose: glycoprotein glucosyltransferase 2
UDP-glucose: glycoprotein glucosyltransferase 8
Cytochrome P450 2C9
Carbonic anhydrase 5B
Serine/threonine-protein kinase Chk2
Mitogen-activated protein kinase 12
Dual specificity protein phosphatase 7
Tyrosine-protein kinase transmembrane receptor ROR4
Gephyrin
FERM, ARHGEF and pleckstrin domain-containing protein 3
Glycosyltransferase 8 domain-containing protein 3
UDP-glucose: glycoprotein glucosyltransferase 3
UDP-glucose: glycoprotein glucosyltransferase 6
Cytochrome P450 2C10
UDP-glucose: glycoprotein glucosyltransferase 4
FERM, ARHGEF and pleckstrin domain-containing protein 4
UDP-glucose: glycoprotein glucosyltransferase 5
Epiplakin
FGF binding protein
Prothymosin alpha
Epiregulin

IV. Methods of Making an Anti-Fibrotic or Wound Healing Peptide from a Regenerative Species Protein

To derive the peptides of the instant disclosure, peptide motifs predicted to be functional as intracellular or extracellular ligands in an anti-fibrotic treatment are first selected from a regenerative species protein expressed during tissue regeneration, and then analyzed for sequence conservation among species, e.g., between axolotl and other regenerative species, or between axolotl and human sequences.

To predict peptide motifs, domain sequence, structure, and species conservation information may be obtained from a number of sources, e.g., PROSITE (www.prosite.expasy.org), PFAM (www.pfam.xfam.org), and NCBI-CDD (www.ncbi.nlm.nih.gov/Structure/cdd/cdd.shtml databases). In addition, various methods known in the art may be used to determine the cross-species homology of the peptide motifs. For example, homology between axolotl and human peptide motif sequences may be determined by comparing the percent sequence identity between the putative axolotl peptide sequence and the human sequence.

Percent sequence identity, e.g., between an axolotl and a human sequence, refers to the percentage of exact matching residues in an alignment of one sequence to another sequence, such as an alignment generated by a BLAST algorithm or other alignment algorithms known in the art. Identity may be calculated based on an alignment of a first full-length sequence, e.g., full-length peptide or protein and a second full-length sequence, e.g., another full-length peptide or protein. Identity may also be calculated based on a partial alignment of a first sequence and a second sequence, if the second sequence is longer than the first sequence. Identity may also be calculated based on a partial alignment of a sequence provided herein and a reference sequence, if the second is shorter than the first sequence. Thus, when aligning two sequences, according to the aforementioned, a query sequence “shares at least x % identity to” a subject sequence if in the alignment of the two sequences, at least x % (rounded down) of the residues in the subject sequence are aligned as an exact match to a corresponding residue in the query sequence, wherein the numerator is the number of exact matches and the denominator is the length of the query sequence. In some embodiments, the denominator may alternatively be the length of the query sequence minus any gaps of two or more non-matching residues. Where the subject sequence has variable positions (e.g., residues denoted X), an alignment to any residue in the query sequence is counted as a match.

As provided herein, a peptide motif may also be selected based on the length or molecular weight of the peptide. In some aspects, the peptide motifs selected from the regenerative protein are between about 5 to about 9 amino acids in length. In some aspects, the peptide motifs selected from the regenerative protein are between about 10 to about 20 amino acids in length. In some aspects, the peptide motifs selected from the regenerative protein are between about 21 to about 51 amino acids in length. In some aspects, the peptide motifs selected from the regenerative protein are between about 51 to about 100 amino acids in length. In some aspects, the peptide motifs selected from the regenerative protein are greater than 100 amino acids in length. In some aspects, the peptide motifs selected from the regenerative protein disclosed herein are between about 2 to about 4 amino acids in length.

Once a peptide motif is selected from a protein sequence as potentially active and conserved among two or more species, the peptide may be modified to improve functionality. In some aspects, the peptide may be modified to improve one or more of further cross-species homology, tissue penetration, peptide stability, and/or affinity to human proteases.

To improve cross-species homology of an axolotl peptide motif to that of a human sequence, for example, human sequence amino acids may be added to the conserved motif and/or to the variable portions of the motif. As a demonstrative example, consider the following protein sequence from an axolotl regenerative protein in which the peptide motif was first selected and then improved to increase homology with the homologous human sequence.

The axolotl regenerative protein sequence of matrix metalloproteinase 2 is as follows (selected peptide motif in bold):

(SEQ ID NO: 2)
GGHRAAGSLSPIQERTRRTREEQNWQTKMRTSTVLHAAGFLLRLLLVNVL
VFQDTIAAPSPIIKFPGDSTPKTEKELAVQYLNKFYGCPKEKCNLFVLKD
TLKKMQKFFGLPETGELDQNTIETMKKPRCGNPDVANYNFFPRKPKWEKN
HITYRIVGYTTDLDTETVDDAFARAFKVWSDVTPLDFSRLNDGDADIMIN
FGRWEHGDGYPFDGKDGLLAHAFAPGQGIGGDSHFDDDEHWTLGDGQVVK
VKFGNADGEFCKFPFLFGDKEYNSCTDAGRSDGFLWCSTTYNFDSDKKYG
FCPHELLFTLAGNADGEPCKFPFKFQGTSYDSCTTEGRTDGYRWCATTAD
YDTDKKYGFCPETALSTVGGNSEGSPCVFPFTFLGNKYDQCTSSGRSDGK
LWCASTSNYDDDRKWGFCPDQGYSLFLVAAHEFGHALGLEHSDDPGALMA
PIYTYTKNFKLSQDDVSGIQELYGGTSEKPGPGPSPTLGPVTPELCKKDI
TFDALSQIRGEIFLFKDRFIWRTVSPRTRPSGPLLVATFWSEVPDKIDAV
YEAPQEEKTVFFAGNEYWIYSASQLERGYPKRLTALGLSDDVKHVDAAFN
WSKNKKTYIFSGDKFWRYNEVKKKMDPGFPKLIADSWTGVPDDLDAVLDH
SGSGYTYFIKDKYYLKLENESLKIVQIGNMKNDWLGCRPS

The human matrix metalloproteinase 2 protein sequence is as follows (selected peptide motif in bold):

(SEQ ID NO: 3)
MEALMARGALTGPLRALCLLGCLLSHAAAAPSPIIKFPGDVAPKTDKELA
VQYLNTFYGCPKESCNLFVLKDTLKKMQKFFGLPQTGDLDQNTIETMRKP
RCGNPDVANYNFFPRKPKWDKNQITYRIIGYTPDLDPETVDDAFARAFQV
WSDVTPLRFSRIHDGEADIMINFGRWEHGDGYPFDGKDGLLAHAFAPGTG
VGGDSHFDDDELWTLGEGQVVRVKYGNADGEYCKFPFLFNGKEYNSCTDT
GRSDGFLWCSTTYNFEKDGKYGFCPHEALFTMGGNAEGQPCKFPFRFQGT
SYDSCTTEGRTDGYRWCGTTEDYDRDKKYGFCPETAMSTVGGNSEGAPCV
FPFTFLGNKYESCTSAGRSDGKMWCATTANYDDDRKWGFCPDQGYSLFLV
AAHEFGHAMGLEHSQDPGALMAPIYTYTKNFRLSQDDIKGIQELYGASPD
IDLGTGPTPTLGPVTPEICKQDIVFDGIAQIRGEIFFFKDRFIWRTVTPR
DKPMGPLLVATFWPELPEKIDAVYEAPQEEKAVFFAGNEYWIYSASTLER
GYPKPLTSLGLPPDVQRVDAAFNWSKNKKTYIFAGDKFWRYNEVKKKMDP
GFPKLIADAWNAIPDNLDAVVDLQGGGHSYFFKGAYYLKLENQSLKSVKF
GSIKSDWLGC

The selected peptide motif determined based on structural and functional algorithms for this protein is HE{1,3}H, where {1,3} denotes a variable region that can be substituted with 1 to 3 amino acids. To improve human sequence homology, i.e., to “humanize” the sequence, two of the amino acids from the human peptide motif (bolded within the sequence) were used as a prefix. Hence the peptide sequence is AAHEFGH (SEQ ID NO: 96). In some instances, the axolotl sequence may have the exact same sequence for the motif in the prefix (AA) and within the motif itself (FG), however this is not always the case. Both the {1,3} variable region (FG) and the prefix (AA) may differ. In the cases in which the variable region and prefixes differ, different combinations of the axolotl and human sequences are created. One sequence could have an axolotl prefix and the variable region may be filled in with a human sequence, or vice versa.

In another example, a peptide motif was selected for the Mb32 peptide. It is derived from the axolotl protein Retinol dehydrogenase 11 which has a sequence as follows (the selected peptide motif is in bold):

(SEQ ID NO: 4)
MDAYWAFLLAPVFLFYIAAPYIRKFVGGGVCKSTAKLHGKVVVITGANTG
IGKETASDLARRGARVVIACRDVAKGEAAASKIRAETGNEQVVVRKLDLA
DTKSVGEFAERMRKEEERIHILINNAGVMMCPYSKTVDGFEMQLGVNHLG
HFLLTYLLLDVLKKSAPARIVNVSSLAHHFGRIKFEDLQCEKSYQSALAY
CRSKLANILFTRELAKRLKGTQVTVNALHPGSVRSELVRHSFLLSKAWTL
CSFFIKTPKEGAQTNIYCAVAEELESVSGKYFSDCSPAYVSSQGRNDETA
RKLWDVSCRLLGIQWN

The human homologous sequence of the Retinol dehydrogenase 11 protein has the following sequence (the selected peptide motif is in bold):

(SEQ ID NO: 5)
MLVTLGLLTSFFSFLYMVAPSIRKFFAGGVCRTNVQLPGKVVVITGANTG
IGKETARELASRGARVYIACRDVLKGESAASEIRVDTKNSQVLVRKLDLS
DTKSIRAFAEGFLAEEKQLHILINNAGVMMCPYSKTADGFETHLGVNHLG
HFLLTYLLLERLKVSAPARVVNVSSVAHHIGKIPFHDLQSEKRYSRGFAY
CHSKLANVLFTRELAKRLQGTGVTTYAVHPGVVRSELVRHSSLLCLLWRL
FSPFVKTAREGAQTSLHCALAEGLEPLSGKYFSDCKRTWVSPRARNNKTA
ERLWNVSCELLGIRWE

In order to improve the human sequence homology of the axolotl peptide motif (YCRSK (SEQ ID NO: 6)), the human prefix FA is appended and creates the humanized peptide of FAYCRSK (SEQ ID NO: 7). This peptide may be further modified to include a carrier sequence, if desirable, for example based on whether the peptide is thought to be an intracellular or extracellular peptide. For example, a carrier sequence, such as carrier sequence GLRKRLRKFRNK (SEQ ID NO: 1) may be appended on the N-terminus of the peptide. Thus, the axolotl peptide motif sequence may be both improved for human sequence homology and for extracellular location, to create the exemplary Mb32 peptide of FAYCRSKGLRKRLRKFRNK (SEQ ID NO: 8).

Thus, in some aspects, as provided herein the peptide motif is modified to improve cross-species homology, as described above. In some aspects, modifying the peptide includes the substitution, deletion, or addition of one or more amino acid residues. In some aspects, the peptide is modified by adding one or more amino acid residues from a homologous species sequence at the C-terminus of the peptide. In some aspects, the peptide is modified by adding one or more amino acid residues from a homologous species sequence at the N-terminus of the peptide. In some aspects, the peptide is modified to increase the cross-species sequence homology by at least 20%.

Further, as described above, in some aspects, to improve cell penetration a cell penetrating or carrier sequence may be added to the peptide. Any suitable cell penetrating or carrier sequence may be used. In some aspects, the cell penetration sequence of GLRKRLRKFRNK (SEQ ID NO: 1) is added to the C-terminus. In other aspects, the cell penetration sequence GLRKRLRKFRNK (SEQ ID NO: 1) is added to the N-terminus. Generally, cell and tissue penetrating or permeating peptides are less than 40 amino acids long, have a positive net charge, and are amphiphilic. Thus, any cell penetrating peptide with these characteristics may be added to a peptide of the disclosure, to either the C or N-terminus

In some aspects, the effect of the tissue penetration or permeating carrier sequence on a peptide may be determined by measuring the percentage of peptide that penetrates the skin to a desired depth. In some aspects, the measurement is performed with a tape stripping assay, in which each layer of skin is tape stripped and sent for chemical analysis. In some aspects, each layer is sent for HPLC-MS analysis. In some aspects, skin penetration is determined by immunohistochemistry. In some aspects, modifying the peptide improves cell or tissue penetration of the peptide by about 20%, by about 30%, by about 40%, by about 50%, or by about 60%, relative to the peptide without the carrier sequence.

A peptide of the instant disclosure may also be modified to improve its stability and folding. In some aspects, modifying the peptide to improve stability includes the substitution, deletion, or addition of one or more amino acid residues. Various programs to model peptide stability may be used. Similarly, various in vitro assays may be performed to measure peptide stability and folding, including but not limited to: X-ray crystallography, fluorescence spectroscopy, circular dichroism (CD) spectroscopy, nuclear magnetic resonance (NMR) spectroscopy, dual-polarization interferometry, size-exclusion chromatography (SEC), and proteolysis. In some aspects, modifying the peptide improves peptide stability by about 20%, by about 30%, by about 40%, by about 50%, or by about 60%, relative to the peptide without the modification, to improve stability or folding.

In some embodiments, the following parameters may also be considered: secondary structure, amphipathicity, ionic charge (e.g., in some embodiments, cationic charge), polyproline motifs, van der Waals volume, functionality with pseudo amino acids, and hydrophobicity. In some exemplary embodiments, the peptide is less than 10 kDa in size.

In some aspects of the instant disclosure, the peptide is modified to improve affinity to human proteases, either to increase or decrease affinity as needed. In some aspects, modifying the peptide to improve affinity includes the substitution, deletion, or addition of one or more amino acid residues. In some embodiments, the peptide modified to improve affinity to a human protease is digested and the fragments are separated. In some aspects, the fragments are separated by liquid chromatography. In some aspects, modifying the peptide increases affinity to human proteases, thereby increasing digestion of the peptide in an example assay, by about 20%, by about 30%, by about 40%, by about 50%, or by about 60%. In some aspects, modifying the peptide decreases affinity to human proteases, thereby decreasing digestion of the peptide in an example assay by about 20%, by about 30%, by about 40%, by about 50%, or by about 60%. Examples of classes of human proteases, to which affinity may be improved, include but are not limited to: aspartate proteases, cysteine proteases, metalloproteases, serine proteases, and threonine proteases.

In some aspects, a modified peptide as provided herein is a peptide of 2-4 amino acids in length. In some aspects, the peptide provided herein is a peptide of 5-10 amino acids in length. In some aspects, the peptide provided herein is a peptide of 10-15 amino acids in length. In some aspects, the peptide provided herein is a peptide of 5-20 amino acids in length. In some aspects, the peptide provided herein is a peptide of 5-25 amino acids in length. In some aspects, the peptide provided herein is a peptide of 5-50 amino acids in length.

In other contemplated embodiments, a regenerative species regenerative protein, e.g., a full-length or nearly full-length sequence, may be modified by improving one or more of cross-species homology, tissue penetration, protein stability, and/or affinity to human proteases and used as an anti-fibrotic treatment.

### V. Methods of Selecting Peptides for Anti-Fibrotic or Wound Healing Treatments Based on Assay Effects

Once a peptide is selected from a regenerative species regenerative protein sequence, and modified to improve functionality, the peptide may be tested by in vitro and/or in vivo methods to determine whether it could be functional in an anti-fibrotic or wound healing treatment. Measures of anti-fibrotic peptide's efficacy may include for example, their effects on in vitro myofibroblast formation, bacterial infection, inflammation, cell migration, extracellular matrix production, keratin production or cell proliferation. In vivo assays may also be performed, for example to determine if the peptides may regulate the rate and quality of tissue replacement, e.g., healing after a clinically induced wound.

### In Vitro Assays

In vitro assays to test the efficacy of the peptides may include, but are not limited to, fibroblast and keratinocyte cells, two cell types which reside in the skin and are important in the fibrotic response. For example, a myofibroblast transition assay (MFT), a proliferation assay, and a scratch assay may be used to assess the effects of the peptides of the instant disclosure on fibroblast transition to myofibroblasts, fibroblast proliferation, and fibroblast and keratinocyte migration into a wound. An inflammation assay for example, may be used to assess the ability of the peptides to lower the inflammation response in fibroblasts. Antimicrobial assays may also be performed to determine the efficacy of the peptides in regulating infection associated with fibrosis.

Myofibroblast transition (MFT) is the transition of fibroblasts to myofibroblasts. Myofibroblasts produce extracellular matrix, including collagen, during tissue remodeling. However, myofibroblast production of collagen and other extracellular matrix materials may be produced in excess and may be difficult to remodel, leaving behind the fibrous connective tissue known as fibrosis. Thus, the ability to regulate the degree of MFT may enable the control of fibrosis, and one assay of interest is to test the peptides of the instant disclosure for their ability to regulate MFT.

An MFT assay may include for example, assessing the amount of an MFT marker, e.g. alpha-smooth muscle actin (aSMA) in cells treated with a peptide versus a negative control. As described in Example 1, exemplary peptides EA1, EA2, PC1, MBb4.2, MBb7, and MBb32, demonstrated a significant inhibition of MFT of at least 25%. In some embodiments, the peptides of the instant disclosure may inhibit MFT by at least 20%, least 25%, at least 30%, at least 35%, at least 40%, at least 45%, at least 50%, at least 55%, at least 60%, at least 65%, at least 70%, at least 75%, or at least 80% relative to a control.

Fibroblasts have a number of roles in fibrosis and wound healing, including migration into the tissue, tissue remodeling and the release of growth factors important in angiogenesis. Fibroblasts also release signaling molecules important for tissue fibrosis which may include modulation of the function and migration of immune cells, keratinocytes, endothelial cells, and mast cells. Thus, another assay of interest is to test the peptides of the instant disclosure for their efficacy as anti-fibrotic treatments is the regulation of fibroblast proliferation.

A fibroblast proliferation assay may assess the percentage of dividing fibroblasts. As described in Example 1, the exemplary peptides MBa5, MBa4, MBb14, and the combination of P2 and P3 peptides all significantly increased fibroblast proliferation more than 50% relative to the control. In some embodiments, the peptides of the instant disclosure may increase fibroblast proliferation by at least 40%, at least 60%, at least 80%, or at least 100% relative to the control.

Fibroblast migration is another measure of the efficacy of an anti-fibrotic or wound healing treatment, as fibroblast migration is rate-limiting to tissue repair. Thus, another assay of interest is to test the peptides of the instant disclosure for their ability to regulate fibroblast migration, e.g., into a wound. One example of a fibroblast migration assay is to assess the rate of fibroblast migration over time into an in vitro plate of confluent or near confluent cells which have been scraped in the middle to simulate a wound, also known as a “scratch assay.” As described in Example 1, the exemplary peptides, MBb4, MBm6, MBb67, MBWhit13, MBWhit15, MBWhit17, MBWhit18, MBWhit3, significantly increased the rate of fibroblast migration by more than 50% into the wound relative to the negative control. In some embodiments, the peptides of the instant disclosure may increase the rate of fibroblast migration by at least 40%, at least 60%, at least 80%, at least 100%, at least 120%, at least 140%, at least 160%, or at least 180% relative to the control.

Reduction of inflammation of fibroblasts is another measure of the efficacy of an anti-fibrotic or wound healing treatment. Inflammation in fibroblasts leads to the production of the chemokine interleukin-8 (IL-8), which stimulates fibroblast to myofibroblast transition (MFT) thereby enhancing fibrosis. To determine if the peptides of the instant disclosure affect fibroblast response to inflammation and fibroblast IL-8 chemokine production, IL-8 production was measured with lipopolysaccharide induction in the presence of various peptides. As shown in Example 1, MBb11 and MBb28 have a significant effect on IL-8 inhibition in fibroblasts relative to the negative control. Thus, in some embodiments, the peptides of the instant disclosure may inhibit IL-8 or other chemokine production by fibroblasts, by a least 20%, at least 30%, or at least 40% relative to the control.

Keratinocytes represent the major cell type of the outermost of the layers of the skin, known as the epidermis, and are essential for reduction of fibrosis and wound healing in the skin. Thus, another assay of interest is to determine the ability of the peptides of the instant disclosure to regulate the rate of keratinocyte migration, e.g., into a wound. A keratinocyte in vitro migration assay, or “scratch assay” may be performed similarly to that of fibroblasts. As shown in Example 1, MBb11 significantly increased keratinocyte migration by at least 39%. In some embodiments, the peptides of the instant disclosure may increase the rate of keratinocyte migration by at least at least 35%, at least 40%, at least 45%, or at least 50% relative to the control.

A keratinocyte proliferation assay may similarly assess the percentage of dividing keratinocytes. In some aspects, the peptides of the instant disclosure increase keratinocyte proliferation by at least 35%, at least 40%, at least 45%, or at least 50% relative to the control. Keratinocytes also produce pro-inflammatory cytokines (e.g., TNF-α and IL-1) and chemokines (e.g., CXCL1, -5, -8) in response to an epithelial tissue wound, which recruits neutrophils and macrophages. In some aspects of the disclosure, the peptides reduce keratinocyte inflammatory response and improve wound healing. In some embodiments, the peptides of the instant disclosure may inhibit pro-inflammatory chemokine and chemokine production by keratinocytes, by a least 20%, at least 30%, or at least 40% relative to a control.

Infection is another perturbation that causes fibrosis and may delay or inhibit wound healing. To determine if the peptides of the instant disclosure affect the growth of bacteria, and thus may be appropriate as antimicrobial peptides to be used in anti-fibrotic treatments, growth of the following bacterial strains was tested with the exemplary peptides: Staphylococcus aureus: USA300, ST80, JE2, and MRSA col; Streptococcus pneumoniae: DCC1335 and D39; Klebsiella pneumoniae; Pseudomonas aeruginosa; and Acinetobacter baumannii. As shown in Example 1, the Mbb32 peptide had the strongest effect on inhibition of bacterial growth among those peptides tested. Thus, in some embodiments, the peptides of the instant disclosure may bacterial growth, by a least 20%, at least 30%, at least 40%, at least 50%, at least 60%, at least 70%, or at least 80% relative to the control.

In some aspects, the peptides of the disclosure may be assessed by the levels and organization of extracellular matrix, e.g., collagen, elastin, or hyaluronic acid, or of keratin in response to a tissue perturbation, e.g., a wound. Assessment of extracellular matrix production or keratin produced by a cell, e.g., a keratinocyte or fibroblast, or by a tissue may be performed, for example with immunohistochemistry, immunofluorescence, or mass-spectrometry techniques. In some aspects, the effect of a peptide of the disclosure on the levels and/or organization of extracellular matrix or keratin produced by a cell or tissue may be a measure of peptide efficacy.

In some aspects, the peptides of the disclosure may be assessed by additional assays, e.g., RNA-seq assays or DNA damage assays, performed with methods known in the art. An RNA-seq assay in a cell or tissue, for example, may provide information related to the expression of wound healing genes in a particular cell type or tissue in response to a peptide. A DNA damage assay, for example, may provide information about the health of a particular cell or tissue in response to a peptide.

### In Vivo Assays

In vivo assays to determine the rate and quality of healing of a clinically induced wound, may inform whether the peptides affect fibrosis and/or wound healing. In vivo assays may include for example, skin smoothness imaging, a transepidermal water loss (TEWL) assay, or skin redness (erythema) imaging, after a clinically induced dermabrasion procedure.

To determine the effects of the peptides of the instant disclosure on wound healing, skin may be imaged during wound healing. For example, as shown in Example 2, Visioscan imaging after dermabrasion in the presence of peptides p1-p9 was performed. Visioscan imaging allows for the determination of skin topography, e.g., skin smoothness. The effect of the exemplary peptides P2 and P3 on skin smoothness was visible after 8 hours, 24 hours, 48 hours, and 72 hours post-application, and may be quantified by methods known in the art. In some embodiments, the peptides of the instant disclosure may improve skin smoothness by a least 20%, at least 30%, at least 40%, at least 50%, at least 60%, at least 70%, or at least 80% relative to the control.

Skin integrity is an indication of the rate of wound healing. To determine the integrity of the skin during wound healing, a transepidermal water loss (TEWL) assay may be performed. As shown in Example 2, the exemplary peptides P2 and P3 demonstrated the most promising TEWL assay results after 8 hours and 48 hours post application. In some embodiments, the peptides of the instant disclosure may improve a TEWL assay result by at least 20%, at least 40%, at least 60%, at least 80%, at least 100%, at least 120%, at least 140%, or at least 160% relative to the control.

Skin redness is an indication of inflammation and/or the presence of a wound, as well as an aesthetic concern. To determine the effects of the peptides of the instant disclosure on skin redness, also known as erythema, a Chromameter measurement may be performed. As shown in Example 3, each of the exemplary peptides P2 and P3 demonstrated significantly improved erythema in the clinical study relative to the control. Thus, in some embodiments, the peptides of the instant disclosure may improve erythema by at least 20%, at least 40%, at least 60%, at least 80%, at least 100%, at least 120%, at least 140%, or at least 160% relative to the control.

The peptides of the instant disclosure may be assessed for their suitability in an anti-fibrotic treatment by any of the above assays, or other assays known in the art. The peptides may be assessed by one or more assays, and may be assessed either in combination or alone. In some aspects, to determine whether a peptide, or a combination of peptides, e.g., two or more peptides, is functional as an anti-fibrotic treatment, one or more peptides from a subset of peptides may be assessed with the following with a combination of assays.

In some aspects, one or more assays are performed with a peptide, or a combination of peptides, to determine whether a peptide is functional as an anti-fibrotic treatment. In some aspects, the one or more assays are selected from a myofibroblast transition assay (MFT), a scratch assay, a proliferation assay, an inflammation assay, and an anti-microbial assay.

In some aspects, a peptide, or a combination of peptides, is selected when the result of one of the assays is at least 40% greater than the control, or when the results of two or more assays are both at least 25% greater than the control.

In some aspects, a peptide, or a combination of peptides, is selected for use in an anti-fibrotic or wound healing treatment when the result of the MFT assay is at least 25%, at least 30%, at least 35%, at least 40%, at least 45%, at least 50%, at least 55%, at least 60%, at least 65%, at least 70%, at least 75%, or at least 80% greater than the control.

In some aspects, a peptide, or a combination of peptides, is selected for use in an anti-fibrotic or wound healing treatment when the result of the scratch assay is at least 25%, at least 35%, or at least 40% greater than the control when performed on keratinocytes.

In some aspects, a peptide, or a combination of peptides, is selected for use in an anti-fibrotic or wound healing treatment when the result of the scratch assay is at least 40%, at least 60%, at least 80%, at least 100%, at least 120%, at least 140%, at least 160%, or at least 180% greater than the control when performed on fibroblasts.

In some aspects, a peptide, or a combination of peptides, is selected for use in an anti-fibrotic or wound healing treatment when the result of the proliferation assay, e.g., with fibroblasts, is at least 40%, at least 60%, at least 80%, or at least 100% greater than the control.

In some aspects, the one or more peptides are selected for use in an anti-fibrotic or wound healing treatment when the result of the inflammation assay is at least 20% greater than the control, or the result of the anti-microbial assay is at least 20% greater than the control.

In some aspects, the one or more peptides are selected for use in an anti-fibrotic or wound healing treatment when the result of one or more in vivo measurements is improved relative to the control. In some aspects, the in vivo measurements comprise a transepidermal water loss (TEWL) assay or an erythema assay. In some aspects, the result of the TEWL assay or erythema assay is at least 20%, at least 40%, at least 60%, at least 80%, at least 100%, at least 120%, at least 140%, or at least 160% greater than the control.

In other aspects, in vivo measurements of the anti-fibrotic or wound healing efficacy of a peptide of the disclosure on the clinical healing time and/or degree of scarring of the treatment may be determined relative to a control peptide or known molecule when the peptides are administered to any organ of the body, e.g., including but not limited to the lung, kidney, liver, and skin.

In some aspects, the efficacy of a peptide of the disclosure administered to the lung may be determined by clinical tests including but not limited to High-Resolution Computed Tomography (HRCT), Pulmonary Function Tests (PFTs), Bronchoscopy and Lung Biopsy, a Six-Minute Walk Test, and a Positron Emission Tomography (PET) Scan.

In some aspects, the efficacy of a peptide of the disclosure administered to the kidney may be determined by clinical tests including but not limited to a Kidney Biopsy and/or histopathology, Imaging Techniques (Ultrasound, Elastography, MRI), Blood Biomarkers or Urine Biomarkers (e.g., Serum Creatinine, Glomerular Filtration Rate, Proteinuria, TGF-B), Functional Tests (Renal Scintigraphy, Clearance Studies) and Genetic and Molecular Tests (e.g., Transcriptomics and Proteomics).

In some aspects, the efficacy of a peptide of the disclosure administered to the liver may be determined by clinical tests including but not limited to a Liver Biopsy, Non-Invasive Imaging Techniques (e.g., Transient Elastography (FibroScan), Magnetic Resonance Elastography (MRE), Acoustic Radiation Force Impulse (ARFI) Imaging, and Shear Wave Elastography (SWE)), Blood-Based Biomarkers, Serum Tests, Ultrasound, Computed Tomography (CT), Magnetic Resonance Imaging (MRI), Liver Function Tests (e.g., Serum Albumin, Bilirubin, and Prothrombin Time (PT)), and Genetic and Molecular Testing.

In some aspects, the efficacy of a peptide of the disclosure administered to the skin may be determined by Clinical Examination (e.g., Visual Inspection, Palpation, and Functional Assessment). In some aspects, a Skin Biopsy (e.g., Histological Examination) is performed where a small sample of skin tissue is taken and examined, e.g., under a microscope. The biopsy may be stained with a dye (e.g., Masson's trichrome) to highlight collagen and other fibrotic tissues. The extent of fibrosis may be graded using histological scoring systems. In some aspects, the thickness of the dermis, the density of collagen fibers, and the presence of inflammatory cells may be assessed to determine the efficacy of the peptide.

In some aspects, the efficacy of a peptide of the disclosure administered to the skin may be determined by Imaging Techniques, e.g., High-Frequency Ultrasound, Optical Coherence Tomography (OCT), and Magnetic Resonance Imaging (MRI), Elastography (e.g., Cutaneous Elastography, Shear Wave Elastography), Skin Thickness Measurements, Patient-Reported Outcome Measures (PROMs), Advanced Imaging Techniques (e.g., Confocal Microscopy), and/or Molecular Techniques (e.g., Genetic or Molecular Testing).

In other aspects, the effect of a peptide of the disclosure on the clinical healing time and/or degree of scarring of an anti-fibrotic or wound healing treatment on the skin may be assessed in response to a scrape biopsy, full biopsy, punch biopsy, chemical peel, laser peel, micro-needling, tape stripping, burn of the skin, contact dermatitis, psoriasis plaques, or surgery.

In some aspects, in vivo measurements of peptide efficacy are performed to determine the effect of the peptides on one or more factors associated with micro-wound accumulation. In some aspects, collagen, elastin, hyaluronic acid, or keratin production may be assessed. In some aspects, the firmness of the skin, elasticity of the skin, smoothness of the skin, appearance of wrinkles, or the depth of wrinkles are assessed as measurements of in vivo peptide efficacy. Assessment of extracellular matrix production or keratin production may be performed, for example with a biopsy, and subsequent immunohistochemistry, immunofluorescence, or mass-spectrometry techniques. In some aspects, the effect of a peptide of the disclosure on collagen, other extracellular matrix components, or keratin production may be assessed, for example with ultrasound.

In some aspects, the effect of a peptide of the disclosure on firmness and elasticity of the skin may be assessed, for example with a Cutometer. In some aspects, the effect of a peptide of the disclosure on smoothness of the skin or appearance of wrinkles may be assessed, for example with VISIA CR or Visioscan imaging. Further, in some aspects, the effect of a peptide of the disclosure on the depth of wrinkles and skin smoothness may be assessed, for example with PRIMOS imaging.

In some aspects, the effect of a peptide of the disclosure on post inflammatory hyperpigmentation by acne, skin tone unevenness, or melasma, which is marked by darker pigment, can be measured by Chromameter or VISIA CR imaging. In some aspects, the effect of a peptide of the disclosure on deeper pigmented spots below the skin surface can be measured by UV VISA image analysis. In some aspects, the effect of a peptide of the disclosure on acne may be assessed by testing the presence of bacterial porphyrins or determining the bacterial microbiome of the acne.

In other aspects, the effect of a peptide of the disclosure on stretch marks may be assessed with visual grading, thermography or ultrasound. In some aspects, the effect of a peptide of the disclosure on general hydration and brightness of the skin may be of interest. Hydration of the skin may be measured by electrical impedance, providing a measure of the amount of water in the epidermis. In some aspects, the effect of a peptide of the disclosure on skin brightness may be measured by the quantity of light reflected from the skin.

### VI. Peptide Formulations

Provided herein are peptide formulations and compositions derived from regenerative species proteins, and methods of making and use thereof, that seek to reduce fibrosis. A reduction of fibrosis may include scar reduction, restoring key structural compounds to the skin, e.g., collagen, elastin, hyaluronic acid, and keratin, restoring skin barrier function, and reducing inflammation and/or pain. The anti-fibrotic peptide formulations and compositions of the disclosure may be formulated to treat any micro or macro perturbation to a tissue.

Also provided herein are peptide compositions derived from regenerative species proteins that seek to improve wound healing. Improvements to wound healing may include scar reduction, restoring key structural compounds to the skin, e.g., collagen, elastin, hyaluronic acid, and keratin, restoring skin barrier function, and reducing the inflammation and/or pain associated with a wound. The peptide compositions of the disclosure may be formulated to treat any micro or macro wound, resulting from a medical or aesthetic need, including but not limited to age-related skin impact, skin inflammation, skin infection, tissue or organ generation, chronic wound healing, wound healing associated with diseases or disorders, e.g., epithelial disorders, burn treatments, or combinations thereof.

In some aspects the anti-fibrotic peptide formulations and compositions reduce fibrosis for tissue or organ generation, chronic wound healing, or fibrosis associated with diseases or disorders.

In some aspects the anti-fibrotic peptide formulations and compositions reduce fibrosis associated with systemic scleroderma, localized scleroderma (i.e., morphea), eosinophilic fasciitis (i.e., Shulman's syndrome), lipodermatosclerosis, nephrogenic systemic fibrosis (NSF), scleredema, scleromyxedema, diffuse cutaneous systemic sclerosis, chronic graft-versus-host Disease (cGVHD) with skin involvement, dermatofibrosarcoma protuberans (DFSP), sclerotic chronic graft-versus-host disease (cGVHD), lichen sclerosus, calciphylaxis (calcific uremic arteriolopathy), acrodermatitis chronica atrophicans, poikiloderma of civatte, epidermolysis bullosa, lichen planus, keloids, hypertrophic scarring, or necrobiosis lipoidica.

In some aspects, the anti-fibrotic peptide formulations and compositions reduce fibrosis associated with epidermolysis bullosa, localized scleroderma, or necrobiosis lipoidica.

In some aspects, the anti-fibrotic peptide formulations and compositions treat fibrosis associated with keloids, hypertrophic scarring, systemic scleroderma, lichen planus, lichen sclerosus, or lipodermatosclerosis. In some aspects, the reduction of fibrosis reduces the inflammation, thickening, and/or hardening of a tissue.

In some aspects, the anti-fibrotic peptide formulations and compositions treat fibrosis associated with or caused by a disease or disorder that produces fibrotic tissue throughout one or more organs. In some aspects, the anti-fibrotic peptide formulations and compositions treat fibrosis associated with or caused by cancer or a metabolic disorder. In some aspects, the anti-fibrotic peptide formulations and compositions treat fibrosis associated with or caused by a genetic disorder. In some aspects, the anti-fibrotic peptide formulations and compositions treat fibrosis associated with or caused by surgery, injury, infection, or substance use, e.g., alcohol or smoking. In some aspects, the anti-fibrotic peptide formulations and compositions treat fibrosis associated with or caused by exposure to allergens.

In some aspects, the anti-fibrotic peptide formulations and compositions treat fibrosis of the liver. In some aspects the liver fibrosis is hepatic fibrosis or cirrhosis. In some aspects the hepatic fibrosis is due to chronic hepatitis or abuse of alcohol. In some aspects the fibrosis is associated with or caused by liver cancer.

In some aspects, the anti-fibrotic peptide formulations and compositions treat lung fibrosis. In some aspects the lung fibrosis is Idiopathic Pulmonary Fibrosis (IPF). In some aspects the lung fibrosis is associated or caused by Pneumoconiosis. In some aspects the lung fibrosis is associated with or caused by inhalation of dust, asbestosis or silicosis. In some aspects the lung fibrosis is associated with or caused by Chronic Hypersensitivity Pneumonitis. In some aspects the lung fibrosis is associated or caused by exposure to allergens. In some aspects the lung fibrosis is associated with or caused by Cystic Fibrosis. In some aspects the fibrosis is associated with or caused by lung cancer.

In some aspects, the anti-fibrotic peptide formulations and compositions treat cardiac fibrosis. In some aspects the cardiac fibrosis is caused by or associated with a heart condition, e.g., a myocardial infarction and/or heart failure. In some aspects the cardiac fibrosis is associated with or caused by Hypertrophic Cardiomyopathy. In some aspects, the cardiac fibrosis is associated with or caused by a genetic disorder.

In some aspects, the anti-fibrotic peptide formulations and compositions treat kidney fibrosis. In some aspects the kidney fibrosis is associated with or caused by Chronic Kidney Disease (CKD). In some aspects the kidney fibrosis includes interstitial fibrosis. In some aspects the kidney fibrosis is Diabetic Nephropathy. In some aspects the kidney fibrosis is associated with or caused by chronic diabetes.

In some aspects, the anti-fibrotic peptide formulations and compositions treat pancreatic fibrosis. In some aspects, the fibrosis is associated with or caused by Chronic Pancreatitis. In some aspects the pancreatic fibrosis is associated with or caused by Cystic Fibrosis. In some aspects the fibrosis is associated with or caused by pancreatic cancer.

In some aspects, the anti-fibrotic peptide formulations and compositions treat intestinal fibrosis. In some aspects, the intestinal fibrosis is associated with or caused by Crohn's Disease. In some aspects, the fibrosis is associated with or caused by a bowel disease. In some aspects, the intestinal fibrosis is associated with or caused by Radiation Enteritis.

In some aspects, the anti-fibrotic peptide formulations and compositions treat ocular fibrosis. In some aspects, the fibrosis is associated with or caused by Proliferative Vitreoretinopathy. In some aspects the ocular fibrosis is associated with or caused by ocular surgery. In some aspects the ocular fibrosis is associated with or caused by Corneal Fibrosis. In some aspects the ocular fibrosis is associated with or caused by injury or infection.

In some aspects, the anti-fibrotic peptide formulations and compositions treat bone marrow fibrosis. In some aspects, the fibrosis is associated with or caused by Myelofibrosis. In some aspects, the fibrosis is associated with or caused by a blood cell abnormality or blood cancer.

In some aspects, the anti-fibrotic peptide formulations and compositions treat uterine fibrosis. In some aspects, the fibrosis is associated with or caused by Asherman's Syndrome. In some aspects the fibrosis is associated with or caused by endometriosis. In some aspect the fibrosis is ovarian fibrosis. In some aspects, the fibrosis is associated with or caused by uterine or ovarian cancer.

In some aspects, the anti-fibrotic or wound healing peptide formulations and compositions treat age-related skin impact, skin inflammation, skin infection, tissue or organ generation, chronic wound healing, fibrosis associated with diseases or disorders, e.g., epithelial disorders, burn treatments, or combinations thereof.

In some aspects, the anti-fibrotic or wound healing peptide formulations and compositions may be formulated to treat acne, reduce or ameliorate aging, or improve the health or appearance of the skin. The peptide formulations provided herein may, for example, include a skin protectant, wound healing, or acne treatment monograph to produce a peptide formulation. In some aspects, the peptide formulation is applied topically, and self-administered by the subject, or administered in a clinic, medical facility, or by an aesthetician. In some aspects, the peptide formulation is a serum, gel, lotion, cream, ointment, mask, peel, or spray. In some aspects, the formulation is applied with a silicon or allantoin gel. In some aspects, the formulation is applied with a silicon patch.

Exemplary peptides of the disclosure are provided herein in Table 2A. The exemplary peptides of Table 2A may be used with any of the formulations and methods described herein.

TABLE 2A
Exemplary Peptides

| | | SEQ |
|---|---|---|
| Peptide | Sequence | ID NO |
| MBb10 | EYENPGLRKRLRKFRNK | 9 |
| MBb11 | QFCGPCGLRKRLRKFRNK | 10 |
| MBb12 | DPCPLCGLRKRLRKFRNK | 11 |
| MBb14 | ERDLK | 12 |
| MBb1.2 | EEIDA | 13 |
| MBb23 | EELTRGLRKRLRKFRNK | 14 |
| MBb24 | AVHELGH | 15 |
| MBb25 | IAHELGHNLGMSH | 16 |
| MBb28 | PKPQP | 17 |
| MBb2.2 | IAHELGH | 18 |
| MBb31 | FVDADGLRKRLRKFRNK | 19 |
| MBb32 | FAYCRSKGLRKRLRKFRNK | 8 |
| MBb33 | VGDFDGLRKRLRKFRNK | 20 |
| MBb35 | NTCHQCGLRKRLRKFRNK | 21 |
| MBb36 | GESKGLGLRKRLRKFRNK | 22 |
| MBb37 | ENIKKGLRKRLRKFRNK | 23 |
| MBb3.2 | LPRDER | 24 |
| MBb41 | CGGLPGLRKRLRKFRNK | 25 |
| MBb44 | PPSILGLRKRLRKFRNK | 26 |
| MBb4.2 | RVIWA | 27 |
| MBb5 | ASCRC | 28 |
| MBb50 | SSSGLGLRKRLRKFRNK | 29 |
| MBb51 | SAYGVSKGLRKRLRKFRNK | 30 |
| MBb52 | AHSDLGLRKRLRKFRNK | 31 |
| MBb53 | VIGKGG | 32 |
| MBb56 | PRGQQGLRKRLRKFRNK | 33 |
| MBb57 | KHFDSPRGGLRKRLRKFRNK | 34 |
| MBb59 | TSRDEGLRKRLRKFRNK | 35 |
| MBb60 | QLDPDGLRKRLRKFRNK | 36 |
| MBb62 | VHEIQRGLRKRLRKFRNK | 37 |
| MBb63 | SVTWIIGLRKRLRKFRNK | 38 |
| MBb66 | ASLHGNGLRKRLRKFRNK | 39 |
| MBb67 | AQYNPGLRKRLRKFRNK | 40 |
| MBb7 | WVSGL | 41 |
| MBb73 | EKKNPGLRKRLRKFRNK | 42 |
| MBb74 | RWMAP | 43 |
| MBb76 | IGPGLP | 44 |
| MBBey12 | MDQGYPKETVDDFPG | 45 |
| MBBey14 | RNPDSPQ | 46 |
| MBBey24 | HNGILID | 47 |
| MBBey3 | HIWA VVPCIR | 48 |
| MBBey37 | VLFEDVIAEP | 49 |
| MBBey38 | QLFEDVIAEP | 50 |
| MBBey4 | AKEVEKH | 51 |
| MBBey41 | VYFVIKT | 52 |
| EA-1 | CDADKKDYCLFGRCQYIVELETFRCRCQKGYYGER | 53 |
| | CVYLEL | |
| EA-2 | IRTLDCDADKKDYCLFGRCQYIVELETFRCRCQKG | 54 |
| | YYGERCVYLEL | |
| MBal | DCRICS | 55 |
| MBa4 | IDFEDVIAEP | 56 |
| MBa5 | FEDVIAEP | 57 |
| MBa7 | GYRCSKG | 58 |
| MBa9 | ITCPHCQ | 59 |
| MBa10 | PSMELVLW | 60 |
| MBb4 | ERCVY | 61 |
| MBm1 | AATHEFGHSLGLSH | 62 |
| MBm2 | VAAHELGHSLGMGH | 63 |
| MBm6 | LVAAHEVGHALGLGH | 64 |
| P2 | QRLLEAQAAT | 65 |
| P3 | SKPAKAPE | 66 |
| PC-1 | LKEKKPAKEK | 67 |
| PS-2 | SDAAVDTSSEITTKDLKEKKEVVEEAENGR | 68 |
| MBWhit1 | FFLRIHPDGRVDGVR | 69 |
| MBWhit12 | FRSCDLR | 70 |
| MBWhit13 | RSCDLR | 71 |
| MBWhit15 | CFRSCDLR | 72 |
| MBWhit17 | CINTEGGYVCRC | 73 |
| MBWhit18 | CMCAEGYALSRDRKYC | 74 |
| MBWhit3 | FFLRIHPDGRVDGV | 75 |
| MBWhit33 | GDIR VR | 76 |
| MBWhit38 | KTHPGG | 77 |
| MBWhit44 | IGERCQYRDLKWWELR | 78 |
| MBWhit45 | DVLDKRLFWIQYNREG | 79 |
| MBWhit46 | VCDEPKDQTVVGPALAAYR | 80 |
| MBWhit47 | PHKGLFCHFGSPANRKIGV | 81 |
| MBdol4 | IVGLGPWLG | 82 |
| MBdol9 | VGLWKACTARGC | 83 |
| MBdol15 | GFWDIL WHIPHTIKG | 84 |
| MBdol20 | SLSRTLWAWLPRPCKSN | 85 |
| MBdo123 | TFMSHLFCCVQKRCDCSF | 86 |
| P1 | VSITEAMHRNLVD | 87 |
| P6 | ADAAVENAVAPAESPVKDLKEKKEVVEEPAKEKAE | 88 |
| | N | |
| FA-1 | ECVEDVEAIDQRKVAEEYCGESWSSFCTFFFSMLQ | 89 |
| | N | |
| EA-4 | KKCERCVY | 90 |
| EC-1 | CQKGYYGVRCEHLEL | 91 |
| PH-1 | LKEKK | 92 |
| PA-3 | PAKEK | 93 |
| EH-2 | VRCEH | 94 |
| ES-1 | CSSDMNGYCLHGQCIYLVDMSQNYCRCEVGYTGMR | 95 |
| | CEHFFL | |

In some aspects, a peptide of the instant disclosure comprises an amino acid sequence with at least 60%, at least 70%, at least 80%, at least 90%, at least 91%, at least 92%, at least 93%, at least 94%, at least 95%, at least 96%, at least 97%, at least 98%, or at least 99% sequence identity to a sequence of Table 2A, wherein the peptide has at least one modification relative to a wild-type sequence.

In some aspects, an exemplary peptide of the instant disclosure comprises a sequence derived from a regenerative protein of Table 2B, selected from the group consisting of: Epiplakin, FGF-Binding Protein, Tomoregulin-1, Matrilysin, Epiregulin, Cell division cycle-associated 7-like protein, Scaffold attachment factor B1, Retinol dehydrogenase 11, Rho GTPase-activating protein 22, Unconventional myosin-Ie, Unconventional myosin-Ie, RNA-binding protein 45, and Carbonyl reductase [NADPH] 1.

In some aspects, an exemplary peptide of Table 2B, derived from a regenerative species regenerative healing protein of Table 2B comprises an amino acid sequence of P2, P3, MBb5, MBm2, EA-1, EA-2, MBb11, MBb35, MBb7, MBb32, MBb4.2, MBb76, MBb28, MBb36, or MBb51. In some aspects, a peptide of the instant disclosure comprises an amino acid sequence with at least 60%, at least 70%, at least 80%, at least 90%, at least 91%, at least 92%, at least 93%, at least 94%, at least 95%, at least 96%, at least 97%, at least 98%, or at least 99% sequence identity to a peptide of Table 2B, or one or more of P2, P3, MBb5, MBm2, EA-1, EA-2, MBb11, MBb35, MBb7, MBb32, MBb4.2, MBb76, MBb28, MBb36, or MBb51, wherein the peptide has at least one modification relative to a wild-type sequence.

TABLE 2B
Exemplary Regenerative species
regenerative proteins and peptides derived thereof.

| Peptide | Regenerative species regenerative protein |
|---|---|
| P2 | Epiplakin |
| P3 | FGF-Binding Protein |
| MBb5 | Tomoregulin-1 |
| MBm2 | Matrilysin |
| EA-1 | Epiregulin |
| EA-2 | Epiregulin |
| MBb11 | Cell division cycle-associated 7-like protein |
| MBb35 | Cell division cycle-associated 7-like protein |
| MBb7 | Scaffold attachment factor B1 |
| MBb32 | Retinol dehydrogenase 11 |
| MBb4.2 | Rho GTPase-activating protein 22 |
| MBb76 | Unconventional myosin-Ie |
| MBb28 | Unconventional myosin-Ie |
| MBb36 | RNA-binding protein 45 |
| MBb 51 | Carbonyl reductase [NADPH] 1 |

In some aspects, an exemplary peptide comprises and amino acid sequence of P2, P3, MBb4.2, MBb5, MBm2, EA1, EA2, PC-1, MBb11, MBb35, MBb7, MBb32, MBb4.2, MBb76, MBb28, MBb36, MBa5, MBa4, MBb14, or MBb 51, eMBb4, MBm6, MBb67, MBWhit13, MBWhit15, MBWhit17, MBWhit18, MBWhit3, or MBb28. In some aspects, a peptide of the instant disclosure comprises an amino acid sequence with at least 60%, at least 70%, at least 80%, at least 90%, at least 91%, at least 92%, at least 93%, at least 94%, at least 95%, at least 96%, at least 97%, at least 98%, or at least 99% sequence identity to a peptide of P2, P3, MBb4.2, MBb5, MBm2, EA1, EA2, PC-1, MBb11, MBb35, MBb7, MBb32, MBb4.2, MBb76, MBb28, MBb36, MBa5, MBa4, MBb14, or MBb 51, eMBb4, MBm6, MBb67, MBWhit13, MBWhit15, MBWhit17, MBWhit18, MBWhit3, or MBb28.

In some aspects, the instant disclosure provides compositions comprising one or more peptides and one or more adjuvants, delivery systems, or excipients. In some embodiments, a composition of the instant disclosure comprises a delivery adjuvants or delivery systems, e.g., aluminum salts, calcium phosphate, incomplete Freund's adjuvant, MF59, cochleates, virus-like particles virosomes, polylatic acid (PLA) or poly[lactide-coglycolide] (PLG).

In some embodiments the anti-fibrotic peptide formulations and compositions includes an excipient, or carrier, e.g., an aqueous carrier. A variety of aqueous carriers can be used, e.g., buffered saline. The compositions may contain pharmaceutically acceptable auxiliary substances as those required to approximate physiological conditions such as pH and buffering agents, toxicity countering agents, e.g., disodium phosphate dihydrate, monosodium phosphate, sodium acetate, sodium chloride, potassium chloride, calcium chloride, hydrochloric acid, sodium hydroxide, L-histidine, L-histidine hydrochloride, and sodium lactate. The concentration of active agent in these formulations can vary and are selected based on fluid volumes, viscosities, and body weight in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)). The compositions may contain pharmaceutically acceptable auxiliary substances such as those that contribute to the stability and activity of the pharmacologically active agent or agents, including but not limited to trehalose, sucrose, or other sugars and polysorbate 20, polysorbate 60, polysorbate 80 or other emulsifiers or stabilizers. In some aspects, the formulation comprises a skin penetration enhancer.

In some aspects, the anti-fibrotic peptide formulations or compositions are formulated together with a skin protectant, wound healing, or acne treatment monograph to produce a peptide formulation. In some embodiments, the monograph consists of Allantoin 0.5% or Dimethicone 2%.

In some aspects, the formulation comprises one or more of salicylic acid, benzoyl peroxide, resorcinol, resorcinol monoacetate, or sulfur. In some aspects, the formulation comprises benzoyl peroxide in 2.5-10%; resorcinol in 2% when combined with sulfur; resorcinol monoactetate in 3%, when combined with sulfur; salicylic acid in 0.5 to 2%; sulfur in 3-10%; or sulfur in 3-8% when combined with resorcinol or resorcinol monoacetate. In some aspects, the formulation comprises resorcinol or resorcinol monoacetate combined with sulfur.

In some aspects, the formulation may comprise one or more of Phenethyl Alcohol, Pentylene Glycol, Propanediol, Petrolatum, Vitis Vinifera (Grape) Seed Oil, Dimethicone, Steareth-2, Steareth-20, or Cetyl Alcohol.

### VII. Methods of Treatment

The peptides of the instant disclosure may be used in in anti-fibrotic therapies of all types, including but not limited to the treatment of tissue regeneration in response to inflammation, wounds, disease, or infection. Anti-fibrotic treatments may include reduction of scar formation; restoring key structural compounds to the tissue, e.g., collagen, elastin, hyaluronic acid, and keratin; restoring epithelial barrier function; reducing the pain of the fibrosis; reducing the disease causing the fibrosis; and reducing the underlying inflammation, wounds, or infection causing the fibrosis.

The peptides of the instant disclosure may also be used in wound healing therapies of all types. A peptide of the disclosure may be formulated as a wound healing treatment to aid in replacement of native tissue in humans during the wound healing process. Improvements to wound healing may include scar reduction, restoring key structural compounds to the skin, e.g., collagen, elastin, hyaluronic acid, and keratin, restoring skin barrier function, and/or reducing the inflammation and/or pain associated with a wound. As provided herein, a “wound” refers to any acute or accumulated skin impact, including but not limited to those resulting from physical trauma, infection, burns, inflammation, surgery, or disease.

In some aspects, a peptide formulation of the disclosure, e.g., comprising or more of Table 2A or Table 2B, may be administered to a subject or by the subject to treat fibrosis in systemic scleroderma, localized scleroderma (i.e., morphea), eosinophilic fasciitis (i.e., Shulman's syndrome), lipodermatosclerosis, nephrogenic systemic fibrosis (NSF), scleredema, scleromyxedema, diffuse cutaneous systemic sclerosis, chronic graft-versus-host Disease (cGVHD) with skin involvement, dermatofibrosarcoma protuberans (DFSP), sclerotic chronic graft-versus-host disease (cGVHD), lichen sclerosus, calciphylaxis (calcific uremic arteriolopathy), acrodermatitis chronica atrophicans, poikiloderma of civatte, epidermolysis bullosa, lichen planus, keloids, hypertrophic scarring, and necrobiosis lipoidica.

In some aspects, a peptide formulation of the disclosure, e.g., comprising or more of Table 2A or Table 2B, is administered to a subject or by the subject to treat fibrosis in epidermolysis bullosa, localized scleroderma, or necrobiosis lipoidica.

In some aspects, a peptide formulation of the disclosure, e.g., comprising or more of Table 2A or Table 2B, is administered to a subject or by the subject to treat fibrosis in keloids, hypertrophic scarring, systemic scleroderma, lichen planus, lichen sclerosus, or lipodermatosclerosis. In some aspects, the reduction of fibrosis reduces the inflammation, thickening, and/or hardening of a tissue.

In some aspects, a peptide formulation of the disclosure, e.g., comprising or more of Table 2A or Table 2B, may be administered to a subject or by the subject to treat fibrosis in a disease or disorder that produces fibrotic tissue throughout one or more organs. In some aspects the fibrosis is associated with or caused by cancer or a metabolic disorder. In some aspects the fibrosis is associated with or caused by a genetic disorder. In some aspects the fibrosis is associated with or caused by surgery, injury, infection, or substance use, e.g., alcohol or smoking. In some aspects the fibrosis is associated with or caused by exposure to allergens.

In some aspects, a peptide formulation of the disclosure, e.g., comprising or more of Table 2A or Table 2B, may be administered to a subject or by the subject to treat fibrosis of the liver. In some aspects the liver fibrosis is hepatic fibrosis or cirrhosis. In some aspects the hepatic fibrosis is due to chronic hepatitis or abuse of alcohol. In some aspects the fibrosis is associated with or caused by liver cancer.

In some aspects, a peptide formulation of the disclosure, e.g., comprising or more of Table 2A or Table 2B, may be administered to a subject or by the subject to treat fibrosis of the lung. In some aspects the lung fibrosis is Idiopathic Pulmonary Fibrosis (IPF). In some aspects the lung fibrosis is associated or caused by Pneumoconiosis. In some aspects the lung fibrosis is associated with or caused by inhalation of dust, asbestosis or silicosis. In some aspects the lung fibrosis is associated with or caused by Chronic Hypersensitivity Pneumonitis. In some aspects the lung fibrosis is associated or caused by exposure to allergens. In some aspects the lung fibrosis is associated with or caused by Cystic Fibrosis. In some aspects the fibrosis is associated with or caused by lung cancer.

In some aspects, a peptide formulation of the disclosure, e.g., comprising or more of Table 2A or Table 2B, may be administered to a subject or by the subject to treat cardiac fibrosis. In some aspects the cardiac fibrosis is caused by or associated with a heart condition, e.g., a myocardial infarction and/or heart failure. In some aspects the cardiac fibrosis is associated with or caused by Hypertrophic Cardiomyopathy. In some aspects, the cardiac fibrosis is associated with or caused by a genetic disorder.

In some aspects, a peptide formulation of the disclosure, e.g., comprising or more of Table 2A or Table 2B, may be administered to a subject or by the subject to treat kidney fibrosis. In some aspects the kidney fibrosis is associated with or caused by Chronic Kidney Disease (CKD). In some aspects the kidney fibrosis includes interstitial fibrosis. In some aspects the kidney fibrosis is Diabetic Nephropathy. In some aspects the kidney fibrosis is associated with or caused by chronic diabetes.

In some aspects, a peptide formulation of the disclosure, e.g., comprising or more of Table 2A or Table 2B, may be administered to a subject or by the subject to treat pancreatic fibrosis. In some aspects, the fibrosis is associated with or caused by Chronic Pancreatitis. In some aspects the pancreatic fibrosis is associated with or caused by Cystic Fibrosis. In some aspects the fibrosis is associated with or caused by pancreatic cancer.

In some aspects, a peptide formulation of the disclosure, e.g., comprising or more of Table 2A or Table 2B, may be administered to a subject or by the subject to treat intestinal fibrosis. In some aspects, the intestinal fibrosis is associated with or caused by Crohn's Disease. In some aspects, the fibrosis is associated with or caused by a bowel disease. In some aspects, the intestinal fibrosis is associated with or caused by Radiation Enteritis.

In some aspects, a peptide formulation of the disclosure, e.g., comprising or more of Table 2A or Table 2B, may be administered to a subject or by the subject to treat ocular fibrosis. In some aspects, the fibrosis is associated with or caused by Proliferative Vitreoretinopathy. In some aspects the ocular fibrosis is associated with or caused by ocular surgery. In some aspects the ocular fibrosis is associated with or caused by Corneal Fibrosis. In some aspects the ocular fibrosis is associated with or caused by injury or infection.

In some aspects, a peptide formulation of the disclosure, e.g., comprising or more of Table 2A or Table 2B, may be administered to a subject or by the subject to treat bone marrow fibrosis. In some aspects, the fibrosis is associated with or caused by Myelofibrosis. In some aspects, the fibrosis is associated with or caused by a blood cell abnormality or blood cancer.

In some aspects, a peptide formulation of the disclosure, e.g., comprising or more of Table 2A or Table 2B, may be administered to a subject or by the subject to treat uterine fibrosis. In some aspects, the fibrosis is associated with or caused by Asherman's Syndrome. In some aspects the fibrosis is associated with or caused by endometriosis. In some aspect the fibrosis is ovarian fibrosis. In some aspects, the fibrosis is associated with or caused by uterine or ovarian cancer.

In some aspects, a peptide formulation of the disclosure, e.g., comprising or more of Table 2A or Table 2B, is administered to a subject or by the subject to prevent or reduce scar formation, to restore key structural compounds to the skin, e.g., collagen, elastin, hyaluronic acid, and keratin, to restore skin barrier function, to reduce the inflammation and/or pain associated with a wound, and/or to minimize healing time. In some aspects, a peptide formulation of the disclosure, e.g., comprising or more of Table 2A or Table 2B, is applied topically as a serum gel, lotion, cream, ointment, mask, peel, or spray.

In some aspects, a peptide formulation of the disclosure, e.g., comprising one or more of Table 2A or Table 2B, is administered to a subject to prevent or reduce scar formation.

In some aspects, a peptide formulation of the disclosure, e.g., comprising one or more of Table 2A or Table 2B, is administered to a subject to restore key structural compounds to the skin, e.g., collagen, elastin, hyaluronic acid, and keratin, to the skin.

In some aspects, a peptide formulation of the disclosure, e.g., comprising one or more of Table 2A or Table 2B, is administered to a subject to restore skin barrier function.

In some aspects, a peptide formulation of the disclosure, e.g., comprising one or more of Table 2A or Table 2B, is administered to a subject to reduce inflammation and/or pain.

In some aspects, a peptide formulation of the disclosure, e.g., comprising one or more of Table 2A or Table 2B, is administered to a subject post-surgery to prevent and/or reduce scarring, to reduce inflammation and/or pain, and/or to reduce the healing time of the wound.

In some aspects, a peptide formulation of the disclosure, e.g., comprising one or more of Table 2A or Table 2B, is administered to reduce fibrosis in response to a scrape biopsy, full biopsy, or punch biopsy.

In some aspects, a peptide formulation of the disclosure, e.g., comprising one or more of Table 2A or Table 2B, is administered to reduce fibrosis in response to a skin ulcer.

In some aspects, a peptide formulation of the disclosure, e.g., comprising one or more of Table 2A or Table 2B, is formulated to reduce fibrosis of a burn. In some aspects, a peptide formulation of the disclosure, e.g., comprising one or more of Table 2A or Table 2B, is formulated to prevent or reduce scar formation, to restore key structural compounds to the skin, e.g., collagen, elastin, hyaluronic acid, and keratin, to restore skin barrier function, to reduce the inflammation and/or pain associated with a burn, and/or to minimize healing time.

In some aspects, a peptide formulation of the disclosure, e.g., comprising one or more of Table 2A or Table 2B, is administered to facilitate the generation or regeneration of an organ or tissue by reducing the scar formation, bacterial infection, and/or inflammation at the site of tissue growth.

In some aspects, a peptide formulation of the disclosure, e.g., comprising one or more of Table 2A or Table 2B, is administered to increase cell migration into a wound and/or cell proliferation at the site of the wound.

In some aspects, a peptide formulation of the disclosure, e.g., comprising one or more of Table 2A or Table 2B, is administered to reduce inflammation. In some aspects, the inflammation is associated with a dermatological treatment, e.g., micro-needling, laser, chemical peel, or dermabrasion.

In some aspects, a peptide formulation of the disclosure, e.g., comprising one or more of Table 2A or Table 2B, is administered to heal acne, reduce or ameliorate aging, or improve the health or appearance of the skin.

In some aspects, a peptide formulation of the disclosure, e.g., comprising one or more of Table 2A or Table 2B, is administered to improve collagen production, firmness of the skin, elasticity of the skin, smoothness of the skin, appearance of wrinkles, depth of wrinkles, hyperpigmentation, acne scarring, skin tone evenness, melasma, skin cell turnover, stretch marks, cellulite, skin surface hydration, or skin brightness.

The treatments provided herein are contemplated to treat humans, however any mammal without the capability of completely regenerating tissue may be treated.

The following exemplary embodiments are not intended to be limiting, but rather illustrative of the disclosure.

### EXEMPLARY EMBODIMENTS

Embodiment I-1. A method of synthesizing a peptide for wound healing comprising:
i) selecting a peptide sequence from a wound healing protein sequence from a regenerative species; and
ii) modifying the peptide sequence to improve one or more of cross-species homology, tissue penetration, peptide stability, and/or affinity to human proteases; and
iii) synthesizing the peptide;

wherein the peptide regulates one or more of: myofibroblast formation, bacterial infection, inflammation, cell migration, extracellular matrix production, keratin production, or cell proliferation.

Embodiment I-2. The method of embodiment I-1, wherein the wound healing protein is an extracellular protein.

Embodiment I-3. The method of embodiment I-1, wherein the wound healing protein is an intracellular protein.

Embodiment I-4. The method of embodiment I-1, wherein the modifying to improve cross-species homology improves the sequence homology between sequences of a regenerative species and a human.

Embodiment I-5. The method of embodiment I-1, wherein the modifying to improve cross-species homology improves the sequence homology between sequences of two or more regenerative species.

Embodiment I-6. The method of embodiment I-1, wherein the regenerative species is selected from axolotl, spiny mouse, starfish, planarian, and zebrafish.

Embodiment I-7. The method of embodiment I-1, wherein the extracellular matrix comprises one or more of collagen, elastin, or hyaluronic acid.

Embodiment I-8. The method of embodiment I-1, wherein the modifying to improve cross-species homology includes adding one or more amino acid residues from a homologous sequence at the C-terminus or N-terminus of the peptide fragment or modifying the amino acid sequence of the peptide to increase the cross-species sequence homology by at least 20%.

Embodiment I-9. The method of embodiment I-1, wherein the modifying to improve tissue penetration of the peptide includes adding a peptide sequence of less than 40 amino acids, wherein the peptide sequence has a positive net charge and is amphiphilic.

Embodiment I-10. The method of embodiment I-9, wherein the modifying to improve tissue penetration of the peptide includes adding the peptide sequence of GLRKRLRKFRNK (SEQ ID NO: 1).

Embodiment I-11. The method of embodiment I-9, wherein the modifying improves tissue penetration of the peptide for a wound healing treatment by about 20%, by about 30%, by about 40%, by about 50%, or by about 60%.

Embodiment I-12. The method of embodiment I-1, wherein the modifying to improve peptide stability or the modifying to improve affinity to human proteases includes the substitution, deletion, or addition of one or more peptides.

Embodiment I-13. The method of embodiment I-12, wherein the modifying improves peptide stability by about 20%, by about 30%, by about 40%, by about 50%, or by about 60%.

Embodiment I-14. The method of embodiment I-12, wherein the modifying increases affinity to human proteases by about 20%, by about 30%, by about 40%, by about 50%, or by about 60%.

Embodiment I-15. The method of embodiment I-12, wherein the modifying decreases affinity to human proteases by about 20%, by about 30%, by about 40%, by about 50%, or by about 60%.

Embodiment I-16. The method of embodiment I-1, wherein selection of the peptide sequence from a wound healing protein sequence is based in part on the length or molecular weight of the peptide.

Embodiment I-17. The method of embodiment I-16, wherein the peptide sequence selected from the wound healing protein sequence is between about 5 to about 9 amino acids in length.

Embodiment I-18. The method of embodiment I-16, wherein the peptide sequence selected from the wound healing protein sequence is between about 10 to about 20 amino acids in length.

Embodiment I-19. The method of embodiment I-16, wherein the peptide sequence selected from the wound healing protein sequence is between about 21 to about 51 amino acids in length.

Embodiment I-20. The method of embodiment I-16, wherein the peptide sequence selected from the wound healing protein sequence is between about 51 to about 100 amino acids in length.

Embodiment I-21. The method of embodiment I-16, wherein the peptide sequence selected from the wound healing protein sequence is greater than 100 amino acids in length.

Embodiment I-22. The method of embodiment I-16, wherein the peptide sequence selected from the wound healing proteins sequence is between about 2 to about 4 amino acids in length.

Embodiment I-23. The method of embodiment I-1, wherein the wound healing protein sequence from a regenerative species is one or more of the sequences of Table 1.

Embodiment I-24. A peptide of less than 55 amino acids in length, derived from a wild-type wound healing protein of Table 1, comprising at least one modification relative to the wild-type protein of Table 1, wherein the at least one modification improves one or more of human sequence homology, tissue penetration, peptide stability, and/or affinity to human proteases, and wherein the peptide regulates one or more of: myofibroblast formation, bacterial infection, inflammation, cell migration, extracellular matrix production, keratin production, or cell proliferation.

Embodiment I-25. A modified wound healing protein derived from a wild-type wound healing protein of a regenerative species, comprising at least one modification relative to the wild-type protein sequence, wherein the at least one modification improves one or more of human sequence homology, tissue penetration, peptide stability, and/or affinity to human proteases, and wherein the peptide regulates one or more of: myofibroblast formation, bacterial infection, inflammation, cell migration, extracellular matrix production, keratin production, or cell proliferation.

Embodiment I-26. The modified wound healing protein of embodiment I-25 derived from a wild-type wound healing protein of Table 1.

Embodiment I-27. A method of synthesizing a peptide for wound healing comprising:
i) selecting a peptide sequence from a human wound healing protein sequence; and
ii) modifying the peptide sequence to improve one or more of homology to a regenerative species, tissue penetration, peptide stability, and/or affinity to human proteases; and
iii) synthesizing the peptide;
wherein the peptide regulates one or more of: myofibroblast formation, bacterial infection, inflammation, cell migration, extracellular matrix production, keratin production, or cell proliferation.

Embodiment I-28. A method of selecting one or more peptides from a subset of peptides for use in wound healing comprising:
i) performing one or more assays on the peptides;
ii) comparing a result of the one or more assays of peptides relative to a control, wherein the one or more assays are selected from:
a myofibroblast transition assay (MFT), a scratch assay, a proliferation assay, an inflammation assay, and an anti-microbial assay; and
iii) selecting the peptide for use in wound healing from the subset of peptides when the result of the one or more assays for the one or more given peptide is at least 40% greater than the control, or when the results of two or more assays for the one or more given peptides are both at least 25% greater than the control.

Embodiment I-29. The method of embodiment I-28, comprising selecting the one or more peptides for use in wound healing when:
i) the result of the MFT assay is at least 25%, at least 30%, at least 35%, at least 40%, at least 45%, at least 50%, at least 55%, at least 60%, at least 65%, at least 70%, at least 75%, or at least 80% greater than the control;
ii) the result of the scratch assay is at least 35%, at least 40%, at least 45%, or at least 50% greater than the control when performed on keratinocytes;
iii) the result of the scratch assay is at least 40%, at least 60%, at least 80%, at least 100%, at least 120%, at least 140%, at least 160%, or at least 180% greater than the control when performed on fibroblasts;
iv) the result of the proliferation assay is at least 40%, at least 60%, at least 80%, or at least 100% greater than the control when performed on fibroblasts; or
v) the result of the proliferation assay is at least 35%, at least 40%, at least 45%, or at least 50% greater than the control when performed on keratinocytes.

Embodiment I-30. The method of embodiment I-29, comprising selecting the one or more peptides for use in wound healing when the result of the inflammation assay is at least 20% greater than the control, or the result of the anti-microbial assay is at least 20% greater than the control.

Embodiment I-31. The method of embodiment I-29, comprising selecting the one or more peptides for use in wound healing when the peptide regulates collagen production, elastin production, hyaluronic acid production and/or keratin production as compared to a control in an in vitro assay.

Embodiment I-32. The method of embodiment I-28, wherein the peptide for use in wound healing comprises a sequence derived from a regenerative species wound healing peptide sequence.

Embodiment I-33. The method of embodiment I-32, wherein the peptide for use in wound healing comprises a sequence derived from an axolotl wound healing peptide sequence modified to increase cross-species homology.

Embodiment I-34. The method of embodiment I-29, comprising selecting the one or more peptides for use in wound healing when the result of one or more in vivo measurements is improved relative to the control.

Embodiment I-35. The method of embodiment I-34, wherein the one or more in vivo measurements comprise a transepidermal water loss (TEWL) assay or erythema assay, wherein the result of the TEWL assay or erythema assay is at least 20%, at least 40%, at least 60%, at least 80%, at least 100%, at least 120%, at least 140%, or at least 160% greater than the control.

Embodiment I-36. The method of embodiment I-34, wherein the in vivo measurement is healing time and/or degree of scarring.

Embodiment I-37. The method of embodiment I-34, wherein the in vivo measurement is performed after a scrape biopsy, full biopsy, punch biopsy, chemical peel, laser peel, dermabrasion, micro-needling, tape stripping, burn of the skin, or surgery

Embodiment I-38. The method of embodiment I-34, wherein the in vivo measurement is performed after inducing contact dermatitis.

Embodiment I-39. The method of embodiment I-34, wherein the in vivo measurement is performed on psoriasis plaques.

Embodiment I-40. The method of embodiment I-34, wherein the in vivo measurement measures one or more of collagen production, elastin production, hyaluronic acid production, keratin production, firmness of the skin, elasticity of the skin, smoothness of the skin, appearance of wrinkles, depth of wrinkles, hyperpigmentation, acne scarring, skin tone evenness, melasma, skin cell turnover, stretch marks, cellulite, skin surface hydration, or skin brightness.

Embodiment I-41. A method of selecting a peptide from a subset of peptides for use in wound healing comprising:
i) performing an RNA-seq assay on a peptide;
ii) comparing the result of the assay on the peptide to a control; and
iii) selecting the peptide for use in wound healing from the subset of peptides when the result of the RNA-seq assay is improved relative to the control.

Embodiment I-42. A method of selecting a peptide from a subset of peptides for use in wound healing comprising:
i) performing a DNA damage assay on a peptide;
ii) comparing the result of the assay on the peptide to a control; and
iii) selecting the peptide for use in wound healing from the subset of peptides when the result of the DNA damage assay is improved relative to the control.

Embodiment I-43. The method of embodiment I-40, wherein the control is an inhibitor of MFT.

Embodiment I-44. The method of embodiment I-41, wherein the inhibitor of MFT is EW-7197.

Embodiment I-45. The method of embodiment I-40, wherein the control is an activator of MFT.

Embodiment I-46. The method of embodiment I-43, wherein the of MFT is TGF-beta.

Embodiment I-47. A peptide comprising an amino acid sequence of Table 2, or an amino acid sequence with at least 70% sequence identity thereto, wherein the peptide comprises at least one modification relative to a wild-type sequence.

Embodiment I-48. The peptide of embodiment I-47, wherein the amino acid sequence comprises a sequence of P2, or a sequence with at least 70% sequence identity thereto, with at least one modification relative to the wild-type sequence of Epiplakin; or a sequence of P3, or a sequence with at least 70% sequence identity thereto, with at least one modification relative to the wild-type sequence of FGF-Binding protein.

Embodiment I-49. The peptide of embodiment I-47, wherein the peptide sequence is derived from a regenerative species wound healing protein and is modified to increase cross-species sequence homology.

Embodiment I-50. A composition comprising one or more peptides of embodiment I-47, and one or more adjuvants or excipients.

Embodiment I-51. The composition of embodiment I-50, for use in wound healing.

Embodiment I-52. The composition of embodiment I-51, wherein the wound healing comprises scar prevention or reduction, post-surgical wound healing, tissue or organ generation, reduction of fibrosis, chronic wound healing, epithelial disorders, age-related skin impact, treatment of burns, reduction of inflammation, reduction of infection, or combinations thereof.

Embodiment I-53. The composition of embodiment I-52, wherein the wound healing prevents or reduces scar formation.

Embodiment I-54. The composition of embodiment I-52, wherein the scar formation is post-surgical.

Embodiment I-55. The composition of embodiment I-52, wherein the wound healing is generation or regeneration of an organ or tissue.

Embodiment I-56. The composition of embodiment I-52, wherein wound healing reduces fibrosis.

Embodiment I-57. The composition of embodiment I-52, wherein the chronic wound healing improves pressure ulcers.

Embodiment I-58. The composition of embodiment I-52, wherein the chronic wound healing improves diabetic and/or foot ulcers.

Embodiment I-59. The composition of embodiment I-52, wherein the wound healing heals burns.

Embodiment I-60. The composition of embodiment I-52, wherein the wound healing heals epithelial disorders.

Embodiment I-61. The composition of embodiment I-60, wherein the wound healing heals psoriasis.

Embodiment I-62. The composition of embodiment I-60, wherein the wound healing heals dermatitis

Embodiment I-63. The composition of embodiment I-60, wherein the wound healing heals rosacea.

Embodiment I-64. The composition of embodiment I-60, wherein the wound healing heals acne.

Embodiment I-65. The composition of embodiment I-60, wherein the wound healing heals diaper rash.

Embodiment I-66. The composition of embodiment I-52, wherein the wound healing reduces inflammation.

Embodiment I-67. The composition of embodiment I-66, wherein the wound healing reduces inflammation associated with a dermatological treatment.

Embodiment I-68. The composition of embodiment I-67, wherein the dermatological treatment is laser, micro-needling, or dermabrasion.

Embodiment I-69. The composition of embodiment I-51, wherein the wound healing reduces the pain associated with a wound.

Embodiment I-70. The composition of embodiment I-52, wherein the wound healing reduces age-related skin impact.

Embodiment I-71. The composition of embodiment I-70, wherein the wound healing reduces or ameliorates aging, or improves the health or appearance of the skin.

Embodiment I-72. The composition of embodiment I-70 or I-64, wherein the composition is formulated together with a skin protectant, wound healing, or acne treatment monograph to produce a peptide formulation.

Embodiment I-73. The formulation of embodiment I-72, comprising a skin penetration enhancer.

Embodiment I-74. The formulation of embodiment I-72, wherein the formulation is applied topically as a serum, gel, lotion, cream, ointment, mask, peel, or spray.

Embodiment I-75. The formulation of embodiment I-74, wherein the peptide formulation is applied with a silicon or allantoin gel.

Embodiment I-76. The formulation of embodiment I-75, wherein the formulation is applied with a silicon patch.

Embodiment I-77. The formulation of embodiment I-72, where the formulation comprises one or more of salicylic acid, benzoyl peroxide, resorcinol, resorcinol monoacetate, or sulfur.

Embodiment I-78. A method of treatment comprising:
topically applying a formulation comprising a peptide to an area to be treated, wherein the peptide is derived from a regenerative species wound healing protein, and wherein the peptide is modified to include one or more amino acid residues from the homologous human sequence, wherein the peptide regulates one or more of: myofibroblast formation, bacterial infection, inflammation, cell migration, or cell proliferation.

Embodiment I-79. The method of embodiment I-78, wherein the method of treatment comprises wound healing.

Embodiment I-80. The method of embodiment I-79, wherein the wound healing comprises scar prevention or reduction, post-surgical wound healing, tissue or organ generation, reduction of fibrosis, chronic wound healing, epithelial disorders, age-related skin impact, treatment of burns, reduction of inflammation, reduction of infection, or combinations thereof.

Embodiment I-81. The method of embodiment I-80, wherein the wound healing comprises the prevention or reduction of scar formation.

Embodiment I-82. The method of embodiment I-81, wherein the scar formation is post-surgical.

Embodiment I-83. The method of embodiment I-80, wherein the wound healing generates or regenerates an organ or tissue.

Embodiment I-84. The method of embodiment I-80, wherein the wound healing reduces fibrosis.

Embodiment I-85. The method of embodiment I-80, wherein the chronic wound healing treatment improves pressure ulcers.

Embodiment I-86. The method of embodiment I-80, wherein the treatment heals burns.

Embodiment I-87. The method of embodiment I-80, wherein the wound healing heals epithelial disorders.

Embodiment I-88. The method of embodiment I-87, wherein the wound healing heals dermatitis.

Embodiment I-89. The method of embodiment I-87, wherein the wound healing heals rosacea.

Embodiment I-90. The method of embodiment I-87, wherein the wound healing heals acne.

Embodiment I-91. The method of embodiment I-80, wherein the wound healing treatment reduces inflammation.

Embodiment I-92. The method of embodiment I-91 wherein the inflammation is associated with a dermatological treatment.

Embodiment I-93. The method of embodiment I-92, wherein the dermatological treatment is laser, micro-needling, or dermabrasion.

Embodiment I-94. The method of embodiment I-80, wherein the wound healing reduces age-related skin impact.

Embodiment I-95. The method of embodiment I-94, wherein the wound healing reduces or ameliorates aging, or improves the health or appearance of the skin.

Embodiment I-96. The method of embodiment I-87 or I-94, wherein the peptide is formulated together with a skin protectant, wound healing, or acne treatment monograph to produce a peptide formulation.

Embodiment I-97. The method of embodiment I-96, wherein the peptide formulation further comprises a skin penetration enhancer.

Embodiment I-98. The method of embodiment I-96, wherein the peptide formulation is applied topically as a serum, gel, lotion, cream, ointment, mask, peel, or spray.

Embodiment I-99. The method of embodiment I-98, wherein the peptide formulation is applied with a silicon or allantoin gel.

Embodiment I-100. The method of embodiment I-98, wherein the peptide formulation is applied with a silicon patch.

Embodiment I-101. The formulation of embodiment I-98, where the formulation comprises one or more of salicylic acid, benzoyl peroxide, resorcinol, resorcinol monoacetate, or sulfur.

Embodiment I-102. The method of embodiment I-96, wherein the peptide is a peptide selected from Table 2.

Embodiment I-103. A method of treating wound healing comprising topically applying a formulation comprising a peptide fragment of embodiment I-1.

### Set II:

Embodiment II-1. A method of synthesizing a peptide for an anti-fibrotic treatment comprising:
i) selecting a peptide sequence from a regenerative protein sequence from a regenerative species; and
ii) modifying the peptide sequence to improve one or more of cross-species homology, tissue penetration, peptide stability, and/or affinity to human proteases; and
iii) synthesizing the peptide;
wherein the peptide regulates one or more of: myofibroblast formation, bacterial infection, inflammation, cell migration, extracellular matrix production, keratin production, or cell proliferation.

Embodiment II-2. The method of embodiment II-1, wherein the regenerative protein is an extracellular protein.

Embodiment II-3. The method of embodiment II-1, wherein the regenerative protein is an intracellular protein.

Embodiment II-4. The method of embodiment II-1, wherein the modifying to improve cross-species homology improves the sequence homology between sequences of a regenerative species and a human.

Embodiment II-5. The method of embodiment II-1, wherein the modifying to improve cross-species homology improves the sequence homology between sequences of two or more regenerative species.

Embodiment II-6. The method of embodiment II-1, wherein the regenerative species is selected from axolotl, spiny mouse, starfish, planarian, and zebrafish.

Embodiment II-7. The method of embodiment II-1, wherein the extracellular matrix comprises one or more of collagen, elastin, or hyaluronic acid.

Embodiment II-8. The method of embodiment II-1, wherein the modifying to improve cross-species homology includes adding one or more amino acid residues from a homologous sequence at the C-terminus or N-terminus of the peptide fragment or modifying the amino acid sequence of the peptide to increase the cross-species sequence homology by at least 20%.

Embodiment II-9. The method of embodiment II-1, wherein the modifying to improve tissue penetration of the peptide includes adding a peptide sequence of less than 40 amino acids, wherein the peptide sequence has a positive net charge and is amphiphilic.

Embodiment II-10. The method of embodiment II-9, wherein the modifying to improve tissue penetration of the peptide includes adding the peptide sequence of GLRKRLRKFRNK (SEQ ID NO: 1).

Embodiment II-11. The method of embodiment II-9, wherein the modifying improves tissue penetration of the peptide for an anti-fibrotic treatment by about 20%, by about 30%, by about 40%, by about 50%, or by about 60%.

Embodiment II-12. The method of embodiment II-1, wherein the modifying to improve peptide stability or the modifying to improve affinity to human proteases includes the substitution, deletion, or addition of one or more peptides.

Embodiment II-13. The method of embodiment II-12, wherein the modifying improves peptide stability by about 20%, by about 30%, by about 40%, by about 50%, or by about 60%.

Embodiment II-14. The method of embodiment II-12, wherein the modifying increases affinity to human proteases by about 20%, by about 30%, by about 40%, by about 50%, or by about 60%.

Embodiment II-15. The method of embodiment II-12, wherein the modifying decreases affinity to human proteases by about 20%, by about 30%, by about 40%, by about 50%, or by about 60%.

Embodiment II-16. The method of embodiment II-1, wherein selection of the peptide sequence from a regenerative protein sequence is based in part on the length or molecular weight of the peptide.

Embodiment II-17. The method of embodiment II-16, wherein the peptide sequence selected from the regenerative protein sequence is between about 5 to about 9 amino acids in length.

Embodiment II-18. The method of embodiment II-16, wherein the peptide sequence selected from the regenerative protein sequence is between about 10 to about 20 amino acids in length.

Embodiment II-19. The method of embodiment II-16, wherein the peptide sequence selected from the regenerative protein sequence is between about 21 to about 51 amino acids in length.

Embodiment II-20. The method of embodiment II-16, wherein the peptide sequence selected from the regenerative protein sequence is between about 51 to about 100 amino acids in length.

Embodiment II-21. The method of embodiment II-16, wherein the peptide sequence selected from the regenerative protein sequence is greater than 100 amino acids in length.

Embodiment II-22. The method of embodiment II-16, wherein the peptide sequence selected from the regenerative proteins sequence is between about 2 to about 4 amino acids in length.

Embodiment II-23. The method of embodiment II-1, wherein the regenerative protein sequence from a regenerative species is one or more of the sequences of Table 1.

Embodiment II-24. A peptide of less than 55 amino acids in length, derived from a wild-type regenerative protein of Table 1, comprising at least one modification relative to the wild-type protein of Table 1, wherein the at least one modification improves one or more of human sequence homology, tissue penetration, peptide stability, and/or affinity to human proteases, and wherein the peptide regulates one or more of: myofibroblast formation, bacterial infection, inflammation, cell migration, extracellular matrix production, keratin production, or cell proliferation.

Embodiment II-25. A modified regenerative protein derived from a wild-type regenerative protein of a regenerative species, comprising at least one modification relative to the wild-type protein sequence, wherein the at least one modification improves one or more of human sequence homology, tissue penetration, peptide stability, and/or affinity to human proteases, and wherein the peptide regulates one or more of: myofibroblast formation, bacterial infection, inflammation, cell migration, extracellular matrix production, keratin production, or cell proliferation.

Embodiment II-26. The modified regenerative protein of embodiment II-25 derived from a wild-type regenerative protein of Table 1.

Embodiment II-27. A method of synthesizing a peptide for an anti-fibrotic treatment comprising:
i) selecting a peptide sequence from a human regenerative protein sequence; and
ii) modifying the peptide sequence to improve one or more of homology to a regenerative species, tissue penetration, peptide stability, and/or affinity to human proteases; and
iii) synthesizing the peptide;
wherein the peptide regulates one or more of: myofibroblast formation, bacterial infection, inflammation, cell migration, extracellular matrix production, keratin production, or cell proliferation.

Embodiment II-28. A method of selecting one or more peptides from a subset of peptides for use in an anti-fibrotic treatment comprising:
i) performing one or more assays on the peptides;
ii) comparing a result of the one or more assays of peptides relative to a control, wherein the one or more assays are selected from:
a myofibroblast transition assay (MFT), a scratch assay, a proliferation assay, an inflammation assay, and an anti-microbial assay; and
iii) selecting the peptide for use in an anti-fibrotic treatment from the subset of peptides when the result of the one or more assays for the one or more given peptide is at least 40% greater than the control, or when the results of two or more assays for the one or more given peptides are both at least 25% greater than the control.

Embodiment II-29. The method of embodiment II-28, comprising selecting the one or more peptides for use in an anti-fibrotic treatment when:
i) the result of the MFT assay is at least 25%, at least 30%, at least 35%, at least 40%, at least 45%, at least 50%, at least 55%, at least 60%, at least 65%, at least 70%, at least 75%, or at least 80% greater than the control;
ii) the result of the scratch assay is at least 35%, at least 40%, at least 45%, or at least 50% greater than the control when performed on keratinocytes;
iii) the result of the scratch assay is at least 40%, at least 60%, at least 80%, at least 100%, at least 120%, at least 140%, at least 160%, or at least 180% greater than the control when performed on fibroblasts;
iv) the result of the proliferation assay is at least 40%, at least 60%, at least 80%, or at least 100% greater than the control when performed on fibroblasts; or
v) the result of the proliferation assay is at least 35%, at least 40%, at least 45%, or at least 50% greater than the control when performed on keratinocytes.

Embodiment II-30. The method of embodiment II-29, comprising selecting the one or more peptides for use in an anti-fibrotic treatment when the result of the inflammation assay is at least 20% greater than the control, or the result of the anti-microbial assay is at least 20% greater than the control.

Embodiment II-31. The method of embodiment II-30, wherein the control is an inhibitor of MFT.

Embodiment II-32. The method of embodiment II-31, wherein the inhibitor of MFT is EW-7197.

Embodiment II-33. The method of embodiment II-30, wherein the control is an activator of MFT.

Embodiment II-34. The method of embodiment II-33, wherein the activator of MFT is TGF-beta.

Embodiment II-35. The method of embodiment II-29, comprising selecting the one or more peptides for use in an anti-fibrotic treatment when the peptide regulates collagen production, elastin production, hyaluronic acid production and/or keratin production as compared to a control in an in vitro assay.

Embodiment II-36. The method of embodiment II-28, wherein the peptide for use in an anti-fibrotic treatment comprises a sequence derived from a regenerative species an anti-fibrotic treatment peptide sequence.

Embodiment II-37. The method of embodiment II-36, wherein the peptide for use in an anti-fibrotic treatment comprises a sequence derived from an axolotl an anti-fibrotic treatment peptide sequence modified to increase cross-species homology.

Embodiment II-38. The method of embodiment II-29, comprising selecting the one or more peptides for use in an anti-fibrotic treatment when the result of one or more in vivo measurements is improved relative to the control.

Embodiment II-39. The method of embodiment II-38, wherein the one or more in vivo measurements comprise a transepidermal water loss (TEWL) assay or erythema assay, wherein the result of the TEWL assay or erythema assay is at least 20%, at least 40%, at least 60%, at least 80%, at least 100%, at least 120%, at least 140%, or at least 160% greater than the control.

Embodiment II-40. The method of embodiment II-38, wherein the in vivo measurement is healing time and/or degree of scarring.

Embodiment II-41. The method of embodiment II-38, wherein the in vivo measurement is performed on one or more of lung, kidney, liver, or skin tissue.

Embodiment II-42. The method of embodiment II-41, wherein the in vivo measurement is selected from High-Resolution Computed Tomography (HRCT), Pulmonary Function Tests (PFTs), Bronchoscopy and Lung Biopsy, a Six-Minute Walk Test, and a Positron Emission Tomography (PET) Scan.

Embodiment II-43. The method of embodiment II-41, wherein the in vivo measurement is selected from a Kidney Biopsy, Histopathology, Imaging Techniques, Blood-based Biomarkers, Urine Biomarkers, Kidney Functional Tests, Genetic tests, and Molecular Tests.

Embodiment II-44. The method of embodiment II-41, wherein the in vivo measurement is selected from a Liver Biopsy, Non-Invasive Imaging Techniques, Magnetic Resonance Elastography (MRE), Acoustic Radiation Force Impulse (ARFI) Imaging, Shear Wave Elastography (SWE), Blood-Based Biomarkers, Serum Tests, Ultrasound, Computed Tomography (CT), Magnetic Resonance Imaging (MRI), Liver Function Tests, Genetic testing, and Molecular Testing.

Embodiment II-45. The method of embodiment II-41, wherein the in vivo measurement is selected from a Clinical Examination, Skin Biopsy, and Histological Examination.

Embodiment II-46. The method of embodiment II-45, wherein the thickness of the dermis, the density of collagen fibers, and/or the presence of inflammatory cells are assessed.

Embodiment II-47. The method of embodiment II-41, wherein the in vivo measurement is selected from a High-Frequency Ultrasound, Optical Coherence Tomography (OCT), Magnetic Resonance Imaging (MRI), Elastography, Skin Thickness Measurements, Patient-Reported Outcome Measures (PROMs), Advanced Imaging Techniques, and Molecular Techniques.

Embodiment II-48. The method of embodiment II-38, wherein the in vivo measurement is performed after a scrape biopsy, full biopsy, punch biopsy, chemical peel, laser peel, dermabrasion, micro-needling, tape stripping, burn of the skin, or surgery

Embodiment II-49. The method of embodiment II-38, wherein the in vivo measurement is performed after inducing contact dermatitis.

Embodiment II-50. The method of embodiment II-38, wherein the in vivo measurement is performed on psoriasis plaques.

Embodiment II-51. The method of embodiment II-38, wherein the in vivo measurement measures one or more of collagen production, elastin production, hyaluronic acid production, keratin production, firmness of the skin, elasticity of the skin, smoothness of the skin, appearance of wrinkles, depth of wrinkles, hyperpigmentation, acne scarring, skin tone evenness, melasma, skin cell turnover, stretch marks, cellulite, skin surface hydration, or skin brightness.

Embodiment II-52. A method of selecting a peptide from a subset of peptides for use in an anti-fibrotic treatment comprising:
i) performing an RNA-seq assay on a peptide;
ii) comparing the result of the assay on the peptide to a control; and
iii) selecting the peptide for use in an anti-fibrotic treatment from the subset of peptides when the result of the RNA-seq assay is improved relative to the control.

Embodiment II-53. A method of selecting a peptide from a subset of peptides for use in an anti-fibrotic treatment comprising:
i) performing a DNA damage assay on a peptide;
ii) comparing the result of the assay on the peptide to a control; and
iii) selecting the peptide for use in an anti-fibrotic treatment from the subset of peptides when the result of the DNA damage assay is improved relative to the control.

Embodiment II-54. A peptide comprising an amino acid sequence of Table 2, or an amino acid sequence with at least 70% sequence identity thereto, wherein the peptide comprises at least one modification relative to a wild-type sequence.

Embodiment II-55. The peptide of embodiment II-54, wherein the amino acid sequence comprises a sequence of P2, or a sequence with at least 70% sequence identity thereto, with at least one modification relative to the wild-type sequence of Epiplakin; or a sequence of P3, or a sequence with at least 70% sequence identity thereto, with at least one modification relative to the wild-type sequence of FGF-Binding protein.

Embodiment II-56. The peptide of embodiment II-54, wherein the peptide sequence is derived from a regenerative species an anti-fibrotic treatment protein and is modified to increase cross-species sequence homology.

Embodiment II-57. A composition comprising one or more peptides of embodiment II-54, and one or more adjuvants or excipients.

Embodiment II-58. The composition of embodiment II-57, for use in an anti-fibrotic treatment of fibrosis.

Embodiment II-59. The composition of embodiment II-58, wherein the anti-fibrotic treatment treats systemic scleroderma, localized scleroderma, eosinophilic fasciitis, lipodermatosclerosis, nephrogenic systemic fibrosis (NSF), scleredema, scleromyxedema, diffuse cutaneous systemic sclerosis, chronic graft-versus-host Disease (cGVHD) with skin involvement, dermatofibrosarcoma protuberans (DFSP), sclerotic chronic graft-versus-host disease (cGVHD), lichen sclerosus, calciphylaxis (calcific uremic arteriolopathy), acrodermatitis chronica atrophicans, poikiloderma of civatte, epidermolysis bullosa, lichen planus, keloids, hypertrophic scarring, or necrobiosis lipoidica.

Embodiment II-60. The composition of embodiment II-59, wherein the anti-fibrotic treatment treats epidermolysis bullosa, localized scleroderma, or necrobiosis lipoidica.

Embodiment II-61. The composition of embodiment II-59, wherein the anti-fibrotic treatment treats keloids, hypertrophic scarring, systemic scleroderma, lichen planus, lichen sclerosus, or lipodermatosclerosis.

Embodiment II-62. The composition of embodiment II-58, wherein an anti-fibrotic treatment treats inflammation, thickening, and/or hardening of a tissue.

Embodiment II-63. The composition of embodiment II-58, wherein an anti-fibrotic treatment treats fibrosis associated with or caused by a disease or disorder.

Embodiment II-64. The composition of embodiment II-63, wherein the disease is cancer.

Embodiment II-65. The composition of embodiment II-63, wherein the disease or disorder is a metabolic disorder.

Embodiment II-66. The composition of embodiment II-63, wherein the disorder is a genetic disorder.

Embodiment II-67. The composition of embodiment II-58, wherein the fibrosis is associated with or caused by surgery, injury, infection, or substance use.

Embodiment II-68. The composition of embodiment II-58, wherein the fibrosis is associated with or caused by exposure to allergens.

Embodiment II-69. The composition of embodiment II-58, wherein the fibrosis is liver fibrosis.

Embodiment II-70. The composition of embodiment II-69, wherein the liver fibrosis is hepatic fibrosis or cirrhosis.

Embodiment II-71. The composition of embodiment II-58, wherein the fibrosis is lung fibrosis.

Embodiment II-72. The composition of embodiment II-71, wherein the lung fibrosis is Idiopathic Pulmonary Fibrosis (IPF), Pneumoconiosis, Chronic Hypersensitivity Pneumonitis, or Cystic Fibrosis.

Embodiment II-73. The composition of embodiment II-58, wherein the fibrosis is cardiac fibrosis.

Embodiment II-74. The composition of embodiment II-73, wherein the cardiac fibrosis is caused by or associated with a heart condition.

Embodiment II-75. The composition of embodiment II-58, wherein the fibrosis is kidney fibrosis.

Embodiment II-76. The composition of embodiment II-75, wherein the kidney fibrosis is associated with or caused by Chronic Kidney Disease (CKD) or Diabetic Nephropathy.

Embodiment II-77. The composition of embodiment II-58, wherein the fibrosis is pancreatic fibrosis.

Embodiment II-78. The composition of embodiment II-77, wherein the pancreatic fibrosis is associated with or caused by Chronic Pancreatitis or Cystic Fibrosis.

Embodiment II-79. The composition of embodiment II-58, wherein the fibrosis is intestinal fibrosis.

Embodiment II-80. The composition of embodiment II-79, wherein the intestinal fibrosis is associated with or caused by Crohn's Disease or Radiation Enteritis.

Embodiment II-81. The composition of embodiment II-58, wherein the fibrosis is ocular fibrosis.

Embodiment II-82. The composition of embodiment II-81, wherein the ocular fibrosis is associated with or caused by Proliferative Vitreoretinopathy or Corneal Fibrosis.

Embodiment II-83. The composition of embodiment II-58, wherein the fibrosis is bone marrow fibrosis.

Embodiment II-84. The composition of embodiment II-83 wherein the fibrosis is Myelofibrosis.

Embodiment II-85. The composition of embodiment II-58, wherein the fibrosis is uterine fibrosis.

Embodiment II-86. The composition of embodiment II-85, wherein the fibrosis is associated with or caused by Asherman's Syndrome.

Embodiment II-87. The composition of embodiment II-85, wherein the fibrosis is associated with or caused by endometriosis.

Embodiment II-88. The composition of embodiment II-58, wherein the fibrosis is ovarian fibrosis.

Embodiment II-89. The composition of embodiment II-58, wherein the anti-fibrotic treatment comprises scar prevention or reduction, post-surgical wound healing, tissue or organ generation, chronic wound healing, epithelial disorders, age-related skin impact, treatment of burns, reduction of inflammation, reduction of infection, or combinations thereof.

Embodiment II-90. The composition of embodiment II-58, wherein the anti-fibrotic treatment reduces the pain associated with a wound, inflammation, or infection.

Embodiment II-91. The composition of embodiment II-62, wherein the anti-fibrotic treatment prevents or reduces scar formation.

Embodiment II-92. The composition of embodiment II-91, wherein the scar formation is post-surgical.

Embodiment II-93. The composition of embodiment II-91, wherein the anti-fibrotic treatment treats the generation or regeneration of an organ or tissue.

Embodiment II-94. The composition of embodiment II-91, wherein the anti-fibrotic treatment reduces scarring associated with pressure ulcers, diabetic ulcers, foot ulcers, burns, epithelial disorders, psoriasis, dermatitis, rosacea, acne, or diaper rash.

Embodiment II-95. The composition of embodiment II-62, wherein the anti-fibrotic treatment reduces inflammation.

Embodiment II-96. The composition of embodiment II-95, wherein the anti-fibrotic treatment reduces inflammation associated with a dermatological treatment.

Embodiment II-97. The composition of embodiment II-96, wherein the dermatological treatment is laser, micro-needling, or dermabrasion.

Embodiment II-98. The composition of embodiment II-62, wherein the anti-fibrotic treatment reduces age-related skin impact.

Embodiment II-99. The composition of embodiment II-98, wherein the anti-fibrotic treatment reduces or ameliorates aging, or improves the health or appearance of the skin.

Embodiment II-100. The composition of embodiment II-98 or II-94, wherein the composition is formulated together with a skin protectant, wound healing, or acne treatment monograph to produce a peptide formulation.

Embodiment II-101. The formulation of embodiment II-100, comprising a skin penetration enhancer.

Embodiment II-102. The formulation of embodiment II-100, wherein the formulation is applied topically as a serum, gel, lotion, cream, ointment, mask, peel, or spray.

Embodiment II-103. The formulation of embodiment II-102, wherein the peptide formulation is applied with a silicon or allantoin gel.

Embodiment II-104. The formulation of embodiment II-103, wherein the formulation is applied with a silicon patch.

Embodiment II-105. The formulation of embodiment II-100, where the formulation comprises one or more of salicylic acid, benzoyl peroxide, resorcinol, resorcinol monoacetate, or sulfur.

Embodiment II-106. A method of treatment comprising:
applying a formulation comprising a peptide to an area to be treated, wherein the peptide is derived from a regenerative species regenerative protein, and wherein the peptide is modified to include one or more amino acid residues from the homologous human sequence, wherein the peptide regulates one or more of: myofibrolast differentiation, bacterial infection, inflammation, cell migration, or cell proliferation.

Embodiment II-107. The method of embodiment II-106, wherein the method of treatment comprises an anti-fibrotic treatment that reduces fibrosis.

Embodiment II-108. The method of embodiment II-107, wherein the anti-fibrotic treatment treats systemic scleroderma, localized scleroderma (i.e., morphea), eosinophilic fasciitis (i.e., Shulman's syndrome), lipodermatosclerosis, nephrogenic systemic fibrosis (NSF), scleredema, scleromyxedema, diffuse cutaneous systemic sclerosis, chronic graft-versus-host Disease (cGVHD) with skin involvement, dermatofibrosarcoma protuberans (DFSP), sclerotic chronic graft-versus-host disease (cGVHD), lichen sclerosus, calciphylaxis (calcific uremic arteriolopathy), acrodermatitis chronica atrophicans, poikiloderma of civatte, epidermolysis bullosa, lichen planus, keloids, hypertrophic scarring, or necrobiosis lipoidica.

Embodiment II-109. The method of embodiment II-108, wherein the anti-fibrotic treatment treats epidermolysis bullosa, localized scleroderma, or necrobiosis lipoidica.

Embodiment II-110. The method of embodiment II-108, wherein the anti-fibrotic treatment treats keloids, hypertrophic scarring, systemic scleroderma, lichen planus, lichen sclerosus, or lipodermatosclerosis.

Embodiment II-111. The method of embodiment II-107, wherein the anti-fibrotic treatment treats inflammation, thickening, and/or hardening of a tissue.

Embodiment II-112. The method of embodiment II-107, wherein the anti-fibrotic treatment treats fibrosis associated with or caused by a disease or disorder.

Embodiment II-113. The method of embodiment II-112, wherein the disease is cancer.

Embodiment II-114. The method of embodiment II-112, wherein the disease or disorder is a metabolic disorder.

Embodiment II-115. The method of embodiment II-112, wherein the disorder is a genetic disorder.

Embodiment II-116. The method of embodiment II-107, wherein the anti-fibrotic treatment treats fibrosis associated with or caused by surgery, injury, infection, or substance use.

Embodiment II-117. The method of embodiment II-107, wherein the anti-fibrotic treatment treats fibrosis associated with or caused by exposure to allergens.

Embodiment II-118. The method of embodiment II-107, wherein the anti-fibrotic treatment treats liver fibrosis.

Embodiment II-119. The method of embodiment II-118, wherein the liver fibrosis is hepatic fibrosis or cirrhosis.

Embodiment II-120. The method of embodiment II-107, wherein the anti-fibrotic treatment treats lung fibrosis.

Embodiment II-121. The method of embodiment II-120, wherein the lung fibrosis is Idiopathic Pulmonary Fibrosis (IPF), Pneumoconiosis, Chronic Hypersensitivity Pneumonitis, or Cystic Fibrosis.

Embodiment II-122. The method of embodiment II-107, wherein the anti-fibrotic treatment treats cardiac fibrosis.

Embodiment II-123. The method of embodiment II-122, wherein the cardiac fibrosis is caused by or associated with a heart condition.

Embodiment II-124. The method of embodiment II-107, wherein the anti-fibrotic treatment treats kidney fibrosis.

Embodiment II-125. The method of embodiment II-124, wherein the kidney fibrosis is associated with or caused by Chronic Kidney Disease (CKD) or Diabetic Nephropathy.

Embodiment II-126. The method of embodiment II-107, wherein the anti-fibrotic treatment treats pancreatic fibrosis.

Embodiment II-127. The method of embodiment II-126, wherein the pancreatic fibrosis is associated with or caused by Chronic Pancreatitis or Cystic Fibrosis.

Embodiment II-128. The method of embodiment II-107, wherein the anti-fibrotic treatment treats intestinal fibrosis.

Embodiment II-129. The method of embodiment II-128, wherein the intestinal fibrosis is associated with or caused by Crohn's Disease or Radiation Enteritis.

Embodiment II-130. The method of embodiment II-107, wherein the anti-fibrotic treatment treats ocular fibrosis.

Embodiment II-131. The method of embodiment II-130, wherein the ocular fibrosis is associated with or caused by Proliferative Vitreoretinopathy or Corneal Fibrosis.

Embodiment II-132. The method of embodiment II-107, wherein the anti-fibrotic treatment treats bone marrow fibrosis.

Embodiment II-133. The method of embodiment II-132, wherein the fibrosis is Myelofibrosis.

Embodiment II-134. The method of embodiment II-107, wherein the anti-fibrotic treatment treats uterine fibrosis.

Embodiment II-135. The method of embodiment II-134, wherein the fibrosis is associated with or caused by Asherman's Syndrome.

Embodiment II-136. The method of embodiment II-107, wherein the anti-fibrotic treatment comprises scar prevention or reduction, post-surgical wound healing, tissue or organ generation, chronic wound healing, epithelial disorders, age-related skin impact, treatment of burns, reduction of inflammation, reduction of infection, or combinations thereof.

Embodiment II-137. The method of embodiment II-136, wherein the anti-fibrotic treatment reduces scar formation.

Embodiment II-138. The method of embodiment II-137, wherein the scar formation is post-surgical.

Embodiment II-139. The method of embodiment II-107, wherein the anti-fibrotic treatment treats tissue or organ generation.

Embodiment II-140. The method of embodiment II-137, wherein the anti-fibrotic treatment reduces scarring associated with pressure ulcers, diabetic ulcers, foot ulcers, burns, epithelial disorders, psoriasis, dermatitis, rosacea, acne, or diaper rash.

Embodiment II-141. The method of embodiment II-110, wherein the reduction of fibrosis reduces inflammation.

Embodiment II-142. The method of embodiment II-141 wherein the inflammation is associated with a dermatological treatment.

Embodiment II-143. The method of embodiment II-142, wherein the dermatological treatment is laser, micro-needling, or dermabrasion.

Embodiment II-144. The method of embodiment II-107, wherein the anti-fibrotic treatment reduces age-related skin impact.

Embodiment II-145. The method of embodiment II-107, wherein the reduction of fibrosis reduces or ameliorates aging, or improves the health or appearance of the skin.

Embodiment II-146. The method of embodiment II-145, wherein the peptide is formulated together with a skin protectant, wound healing, or acne treatment monograph to produce a peptide formulation.

Embodiment II-147. The method of embodiment II-146, wherein the peptide formulation further comprises a skin penetration enhancer.

Embodiment II-148. The method of embodiment II-147, wherein the peptide formulation is applied topically as a serum, gel, lotion, cream, ointment, mask, peel, or spray.

Embodiment II-149. The method of embodiment II-148, wherein the peptide formulation is applied with a silicon or allantoin gel.

Embodiment II-150. The method of embodiment II-148, wherein the peptide formulation is applied with a silicon patch.

Embodiment II-151. The formulation of embodiment II-148, where the formulation comprises one or more of salicylic acid, benzoyl peroxide, resorcinol, resorcinol monoacetate, or sulfur.

Embodiment II-152. The method of embodiment II-107, wherein the peptide is a peptide selected from Table 2.

Embodiment II-153. A method of reducing fibrosis comprising applying a formulation comprising a peptide fragment of embodiment II-1.

### EXAMPLES

### Example 1: Methods of Selecting a Peptide for Anti-Fibrotic Therapies Based on Assay Effects: In Vitro Protocols

The peptides provided herein are derived from regenerative proteins of regenerative species. To test whether one or more peptides may be functional as an anti-fibrotic treatment, the peptides were first tested in vitro. This Example describes assays that were used—a myofibroblast transition assay (MFT), a scratch assay, a proliferation assay, an inflammation assay, and an anti-microbial assay.

For each set of assays, the most effective concentration value that was significant was selected as the “representative value.” For many of the peptides, there was no effect at all. Table 8 summarizes the results of the in vitro assays.

### Statistical Analysis

Low replicate and high replicate experiments consisted of a sample size of 3 and 8 respectively. The t-statistic was calculated using un-pooled variances from the replicates. A significance level of p=0.05 was used.

### Myofibroblast Transition Assay (MFT)

The myofibroblast transition (MFT) assay determines the effect of the peptide on inhibiting the transition of fibroblasts to myofibroblasts. The extracellular matrix produced by myofibroblasts may be produced in excess and may not be remodeled during tissue healing, leaving behind the fibrous connective tissue that forms a scar. Thus, the ability to regulate the degree of MFT may enable control of scarring. To determine if the peptides provided herein affect MFT, the following assay was performed.

### Fibroblast Cell Culture

Normal human dermal fibroblasts (HNDF) were cultured in high glucose Dulbecco's modified eagle medium (DMEM) supplemented with 10% fetal bovine serum (FBS) and 5 ml of 10,000 units of penicillin/streptomycin. Cells were cultured on 10 cm plates with media changes every 2 days. Cells were passaged in 0.25% Trypsin/EDTA after achieving 80% confluency.

### Induction of Myofibroblast Transition (MFT)

To induce myofibroblast transition (MFT), normal human dermal fibroblasts (HNDFs) were split from 1-10 cm plates to 5-96 well plates. Cells were then allowed to grow the next day. The following day, cells were serum starved (1% FBS in DMEM) for 4 hours prior to adding 20 ng/ml of TGF-Beta to all wells of the plate. 1000 μM of Vactosertib (TEW-7197/EW-7197) was added to serve as a positive control and different concentrations of peptides were added to serve as the test articles. The cells were then exposed to the compounds for 5 more days before fixation.

### Fixing and Staining (of MFT)

The cells were fixed in paraformaldehyde (PFA) for 10-15 minutes, then washed 2× in PBS. Cells were blocked in 4% BSA for 30 minutes at 23° C. Cells were stained with the primary alpha-smooth muscle actin (aSMA) antibody (1:100) for 1 hour at 23° C. or left out at 4° C. overnight. Cells were rinsed 2× with BSA and then the secondary antibody mix was added: Alexa 488 (1:500) and DAPI (1:1000) for 30 min at 23° C. or 1 hr at 4° C. The plates were covered during staining, then rinsed 2× in BSA and stored in PBS.

### Imaging (of MFT)

Plates were imaged using the Celigo Imaging Cytometer by Nexcelcom. The ratio of fluorescent expression of alpha-smooth muscle actin (aSMA) and DAPI were quantified to determine the amount of aSMA/cell.

The change (normalized to the control) in MFT for each concentration is shown along with the p value for all experiments relative to the control of no peptide.

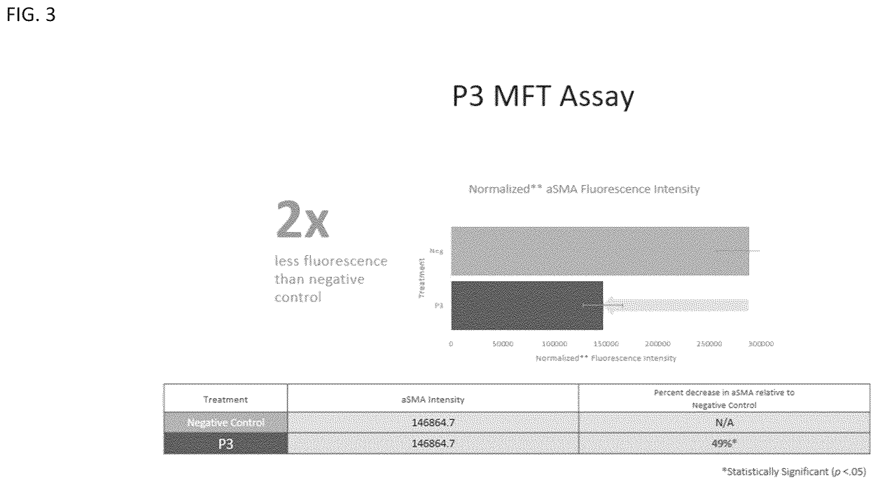

Results: FIG. 3 shows an exemplary peptide P3 aSMA measurement relative to the negative control of no peptide. Fluorescence measurements show that aSMA significantly decreased by 49% relative to the control, indicating a reduction in fibroblast to myofibroblast transition. The results of additional tested peptides at 3 different concentrations are shown in Table 3. Exemplary peptides EA1, EA2, PC1, MBb4.2, MBb7, and MBb32, demonstrated a significant inhibition of MFT of at least 25%.

TABLE 3
Myofibroblast transition (MFT) performed in
replicate (N = 3) at 3 different concentrations of Peptide.

| | | | | | | | Representative |
|---|---|---|---|---|---|---|---|
| | | | | | | | value from |
| | 10 ng/ml | | 100 ng/ml | | 1000 ng/ml | | experiments |
| | (% change | | (% change | | (% change | | (% change |
| | relative to | | relative to | | relative to | | relative |
| Peptide | control) | | control) | | control) | | to control) |
| MBb76 | NS | | −0.03421 | p = (0.01) | −0.18469 | p = (0.009) | −0.18469 p = (0.009) |
| MBb73 | NS | | −0.07558 | p = (0.04) | NS | | −0.07558 p = (0.04) |
| MBb23 | NS | | −0.12033 | p = (0.016) | NS | | −0.12033 p = (0.016) |
| MBb24 | NS | | −0.14194 | p = (0.003) | NS | | −0.14194 p = (0.003) |
| MBb37 | NS | | −0.14264 | p = (0.032) | NS | | −0.14264 p = (0.032) |
| MBb2.2 | −0.15923 | p = (0.038) | −0.14759 | p = (0.035) | −0.19283 | p = (0.021) | −0.19283 p = (0.021) |
| MBb44 | NS | | −0.17509 | p = (0.005) | NS | | −0.17509 p = (0.005) |
| MBb25 | NS | | −0.17686 | p = (0.008) | NS | | −0.17686 p = (0.008) |
| MBb7 | −0.21387 | p = (0.02) | −0.18478 | p = (0.045) | NS | | −0.21387 p = (0.02) |
| MBb36 | −0.07698 | p = (0.028) | −0.19644 | p = (0.002) | NS | | −0.19644 p = (0.002) |
| MBb4.2 | NS | | −0.21133 | p = (0.01) | −0.24164 | p = (0.003) | −0.24164 p = (0.003) |
| MBa1 | NS | | −0.21744 | p = (0.04) | NS | | −0.21744 p = (0.04) |
| MBb28 | NS | | −0.24298 | p = (0.006) | −0.18807 | p = (0.017) | −0.24298 p = (0.006) |
| MBb32 | NS | | −0.25919 | p = (0.039) | −0.30871 | p = (0.039) | −0.30871 p = (0.039) |
| MBb10 | NS | | −0.26941 | p = (0.023) | NS | | −0.26941 p = (0.023) |
| EA-1 | −0.14013 | p = (0.036) | −0.29283 | p = (0.016) | −0.0906 | p = (0.04) | −0.29283 p = (0.016) |
| EA-2 | −0.13431 | p = (0.034) | −0.32409 | p = (0.014) | −0.00716 | p = (0.046) | −0.32409 p = (0.014) |
| MBm2 | −0.42351 | p = (0.007) | −0.32754 | p = (0.018) | −0.20576 | p = (0.025) | −0.42351 p = (0.007) |
| MBb66 | NS | | −0.34462 | p = (0.024) | NS | | −0.34462 p = (0.024) |
| MBb1.2 | NS | | −0.34958 | p = (0.006) | NS | | −0.34958 p = (0.006) |
| MBb11 | NS | | −0.35367 | p = (0.002) | −0.41899 | p = (0.025) | −0.41899 p = (0.025) |
| MBb35 | NS | | −0.3719 | p = (0.01) | −0.40014 | p = (0.016) | −0.40014 p = (0.016) |
| MBb5 | −0.29797 | p = (0.011) | −0.40898 | p = (0.000912) | −0.61616 | p = (0.01) | −0.61616 p = (0.01) |
| MBb12 | NS | | −0.44649 | p = (0.037) | NS | | −0.44649 p = (0.037) |
| PS-2 | −0.43145 | p = (0.052) | −0.49797 | p = (0.048) | NS | | −0.49797 p = (0.048) |
| MBb41 | NS | | −0.61542 | p = (0.03) | −0.2177 | p = (0.051) | −0.61542 p = (0.03) |
| MBa9 | NS | | NS | | −0.31938 | p = (0.02) | −0.31938 p = (0.02) |
| MBb3.2 | NS | | NS | | −0.3874 | p = (0.003) | −0.3874 p = (0.003) |
| MBb51 | −0.5028 | p = (0.042) | NS | | −0.37726 | p = (0.05) | −0.5028 p = (0.042) |
| MBb74 | 0.049754 | p = (0.031) | NS | | −0.30481 | p = (0.019) | −0.30481 p = (0.019) |
| PC-1 | NS | | NS | | −0.47885 | p = (0.038) | −0.47885 p = (0.038) |
| MBWhit1 | NS | | −0.36674 | p = (0.038) | NS | | −0.36674 p = (0.038) |
| MBWhit33 | NS | | −0.31749 | p = (0.034) | −0.35267 | p = (0.032) | −0.35267 p = (0.032) |
| MBWhit38 | NS | | −0.37979 | p = (0.006) | NS | | −0.37979 p = (0.006) |
| MBWhit44 | −0.70201 | p = (0.025) | −0.57528 | p = (0.039) | −0.61106 | p = (0.025) | −0.70201 p = (0.025) |
| MBWhit45 | −0.64714 | p = (0.018) | −0.62653 | p = (0.025) | −0.61362 | p = (0.026) | −0.64714 p = (0.018) |
| MBWhit46 | −0.53399 | p = (0.007) | −0.61911 | p = (0.047) | −0.63276 | p = (0.031) | −0.63276 p = (0.031) |
| MBWhit47 | −0.65751 | p = (0.021) | −0.58962 | p = (0.006) | −0.57551 | p = (0.001) | −0.65751 p = (0.021) |
| MBBey41 | NS | | NS | | −0.69101 | p = (0.021) | −0.69101 p = (0.021) |
| MBBey12 | NS | | −0.66652 | p = (0.007) | −0.44914 | p = (0.018) | −0.66652 p = (0.007) |
| P3 | NS | | NS | | −0.49137 | p = (0.006) | −0.49137 p = (0.006) |
| MBBey 37 | NS | | NS | | −0.28727 | p = (0.018) | −0.28727 p = (0.018) |
| MBBey 38 | −0.30706 | p = (0.007) | NS | | NS | | −0.30706 p = (0.007) |
| MBBey14 | −0.3781 | p = (0.026) | −0.38631 | p = (0.029) | −0.53359 | p = (0.012) | −0.53359 p = (0.012) |
| MBBey24 | −0.05283 | p = (0.005) | 0.068346 | p = (0.022) | 0.274505 | p = (0.035) | −0.05283 p = (0.005) |

| MBBey3 | NS | | −0.14423 | p = (0.037) | NS | −0.14423 p = (0.037) |
|---|---|---|---|---|---|---|
| MBBey4 | −0.22051 | p = (0.021) | −0.44354 | p = (0.007) | NS | −0.44354 p = (0.007) |

(“NS” indicates no statistically significant improvement relative to control;
“—” indicates not tested)

### Fibroblast Cell Proliferation

Fibroblast proliferation is an important component of tissue regeneration. To determine if the peptides of the disclosure affect fibroblast proliferation, HNDFs were grown as described above, and split from 1-10 cm plates to 4-24 well plates. After allowing the cells to grow overnight, normal media was replaced with starvation media (1% FBS in DMEM). 10 ng/ml of fibroblast growth factor (FGF) served as a positive control, untreated cells (starvation media only) served as a negative control, and different concentrations of the peptides as test articles. The cells were then exposed to the peptides for a day before adding 5-Ethynyl-2′-deoxyuridine (EDU) EDU signal marker for 2-4 hours to assess DNA synthesis and cell proliferation.

### Fixing, Permeabilizing, and Staining of Fibroblast EDU

The fibroblasts were fixed in PFA for 10-15 minutes, then washed 2× in 4% BSA. 0.1% Triton X-100 in PBS was added for 20 min at 23° C. to permeabilize the cells. The cells were washed 2× in 4% BSA and the following reaction mixture was added: 0.25% of 488 iFlour azide, 4% CuSO, 10% Sodium Ascorbate in PBS. The cells were incubated at 23° C. for 30 minutes. The cells were washed in BSA 1× and in PBS 1×. The cells were also stained for DAPI for 30 minutes at 23° C. The cells were washed 2× in PBS and maintained in PBS.

### Imaging of Fibroblast EDU

Plates were imaged using the Celigo Imaging Cytometer by Nexcelcom. The amount of fluorescent expression of EDU and DAPI were quantified to determine the amount of EDU/field (and thus the percentage of fibroblast cells dividing).

Because the concentration required to induce an effect with each peptide was not known, each peptide was tested in 3 concentrations: 10 ng/ml, 100 ng/ml, and 1000 ng/ml as shown in Table 4A and Table 4B.

Results: As shown in Table 4A and Table 4B, the peptides MBa5, MBa4, MBb14, and the combination of P2 and P3 peptides all significantly increased fibroblast proliferation more than 50% relative to the control.

TABLE 4A
Fibroblast proliferation performed in replicate (N = 3) at
3 different concentrations of peptide.

| | | | | | Representative |
|---|---|---|---|---|---|
| | | | | | value from |
| | 10 ng/ml | | 100 ng/ml | 1000 ng/ml | experiments |
| | (% change | | (% change | (% change | (% change |
| | relative to | | relative | relative | relative |
| Peptide | control) | | to control) | to control) | to control) |
| MBa5 | 0.5132598724 | (p = 0.004) | NS | NS | 0.5132598724 (p = 0.004) |

| | | | | | |
|---|---|---|---|---|---|
| MBm1 | NS | 0.291893761 | (p = 0.044) | NS | 0.291893761 (p = 0.044) |

| MBa4 | NS | | NS | 0.838378 | p = (0.037) | 0.838378 p = (0.037) |
|---|---|---|---|---|---|---|
| MBa7 | 0.348286 | p = (0.124) | NS | 0.464387 | p = (0.01) | 0.464387 p = (0.01) |

| | | | | | |
|---|---|---|---|---|---|
| MBb14 | 1.094442 | p = (0.029) | NS | NS | 1.094442 p = (0.029) |

| MBb50 | NS | 0.358867 | p = (0.025) | NS | | 0.358867 p = (0.025) |
|---|---|---|---|---|---|---|
| MBb51 | NS | 0.309869 | p = (0.025) | 0.417538 | p = (0.025) | 0.417538 p = (0.025) |

(“NS” indicates no statistically significant improvement relative to control;
“—” indicates not tested)

TABLE 4B
Fibroblast proliferation performed in replicate (N = 8)
at 3 different concentrations of Peptide.

| | 10 ng/ml | 100 ng/ml | 1000 ng/ml |
|---|---|---|---|
| | (% change | (% change | (% change |
| | relative to | relative to | relative to |
| Peptide | control) | control) | control) |
| P2 + P3 | NS | 0.727954 (p = 0.009) | 0.643152 (p = 0.000891) |

(“NS” indicates no statistically significant improvement relative to control;
“—” indicates not tested)

### Fibroblast Scratch Assay

Fibroblast migration into a wound is an important rate-limiting step during tissue regeneration. To determine if the peptides of the disclosure affect the rate of fibroblast migration, scratch assays were performed as follows.

HNDF fibroblasts were grown as described above. HNDFs were split from 1-10 cm plate to 4-24 well plates. Cells were allowed to grow until 90-100% confluent. Cells were serum starved (in 1% FBS in DMEM) for 4 hours before scratching using the Biotek Autoscratch machine. Cells were washed with PBS and the media was replaced with starvation media as a negative control, normal media (10% FBS) as positive control, and starvation media+compound as the test article. Cells were imaged at T=0 to serve as a baseline and fixed at T=48 hours. The images at 48 hours were imaged using phase contrast microscopy or with wheat germ agglutinin (WGA) stain.

Because the concentration required to induce an effect with each peptide was not known, each peptide was tested in 3 concentrations: 10 ng/ml, 100 ng/ml, and 1000 ng/ml as shown in Table 5 and Table 6.

As shown in Table 5, a number of peptides, e.g., MBb4, MBm6, MBb67, MBWhit13, MBWhit15, MBWhit17, MBWhit18, MBWhit3, and P2, significantly increased the rate of fibroblast migration by more than 50% into the wound relative to the negative control.

TABLE 5
Fibroblast scratch assay performed in replicate
(N = 3) at 3 different concentrations of peptide.

| | | | | Representative |
|---|---|---|---|---|
| | | | | value |
| | | | | from |
| | 10 ng/ml | 100 ng/ml | 1000 ng/ml | Experiments |
| | (% change | (% change | (% change | (% change |
| | relative to | relative to | relative to | relative to |
| Peptide | control) | control) | control) | control) |
| MBb4 | NS | 0.593118 p = (0.048) | NS | 0.593118 p = (0.048) |
| MBm6 | 2.120112518 p = (0.044) | NS | 1.236943272 p = (0.046) | 2.120112518 p = (0.044) |
| MBb67 | NS | NS | 0.611981 p = (0.018) | 0.611981 p = (0.018) |
| MBWhit13 | NS | NS | 0.622678 p = (0.004) | 0.622678 p = (0.004) |
| MBWhit15 | 0.52462 p = (0.025) | 0.834406 p = (0.009) | 0.799869 p = (0.024) | 0.834406 p = (0.009) |
| MBWhit17 | NS | NS | 2.019779 p = (0.044) | 2.019779 p = (0.044) |
| MBWhit18 | NS | NS | 0.625143 p = (0.048) | 0.625143 p = (0.048) |
| MBWhit3 | 0.778388 p = (0.001) | 0.607719 p = (0.038) | 1.266761 p = (0.041) | 1.266761 p = (0.041) |
| P2 | 1.01702 p = (0.031) | NS | 1.77926 p = (0.036) | 1.77926 p = (0.036) |

(“NS” indicates no statistically significant improvement relative to control;
“—” indicates not tested)

### Fibroblast Inflammation Assay

Inflammation in fibroblasts leads to the production of the chemokine interleukin-8 (IL-8), which stimulates fibroblast to myofibroblast transition (MFT) thereby enhancing scar formation. To determine if the peptides of the instant disclosure affect fibroblast response to inflammation and fibroblast IL-8 production, the following assays were performed.

### ELISA Assays to Measure Inflammation

Human fibroblasts were grown as described above. HNDFs were plated in 96 well plates at a concentration of 1×10⁴ cells/well and allowed to incubate overnight. The next day cells were dosed with 5 μg/mL of LPS with and without the peptides. The negative control contained only media and vehicle, while the positive control was dosed with lipopolysaccharides (LPS) and dexamethasone (50 μg/ml). After 24 hours, the media was removed to measure IL6 and IL8 cytokine levels using ELISA. A standard curve was plotted in excel. All data were blank subtracted and then fit onto the curve to calculate cytokine concentrations.

As shown in Table 6, MBb11 and MBb28 have a low but significant effect on IL-8 inhibition in fibroblasts relative to the negative control.

TABLE 6
Fibroblast induced chemokine production (IL-8)
induced by lipopolysaccharides (LPS) in the
presence of peptides; performed in replicate (N = 3).

| | 10 ug/ml | 100 ug/ml | 1000 ug/ml |
|---|---|---|---|
| | (% change | (% change | (% change |
| | relative to | relative to | relative to |
| Peptide | control) | control) | control) |
| Mbb32 | — | 4% (p = .054) | 29% (p = 0.035) |
| Mbb1.2 | — | NS | NS |
| EA1 | NS | NS | — |
| EA2 | NS | NS | — |
| PC1 | NS | NS | — |

(“NS” indicates no statistically significant improvement relative to control;
“—” indicates not tested)

### Extracellular Matrix Production

Fibroblast production of extracellular matrix components may also be determined, for example by immunohistochemistry, immunofluorescence, or mass-spectrometry techniques.

### Keratinocyte Assays

Many of the assays performed on fibroblasts may also be performed on keratinocytes, e.g., including cell proliferation assays, scratch assays, inflammation assays, as well as the production of extracellular matrix (ECM), by the methods described above for fibroblasts. Inflammation assays in keratinocytes may measure a keratinocyte-produced chemokine, and determine the effect of a peptide of the disclosure on the production of the pro-inflammatory chemokine. Production of keratin may also be assessed in keratinocytes by the methods described herein for ECM components.

A proliferation assay was performed on immortalized human keratinocytes (HaCaT cells). HaCaT were grown with similar conditions as the HNDF fibroblasts. HaCaTs were serum starved and peptides of the disclosure were added as described above. They were then allowed to grow for a day before being exposed to EDU. Keratinocyte proliferation was measured similarly to the HNDF. As shown in Table 7, a number of the peptides of the disclosure significantly increased keratinocyte proliferation.

TABLE 7
Immortalized human keratinocyte proliferation
assay performed in replicate (N = 8).

| | 1000 ng/ml |
|---|---|
| Peptide | (% change relative to control) |
| MBb62 | 0.074326 p = (0.014) |
| MBb60 | 0.080851 p = (0.02) |
| MBb11 | 0.0841 p = (0.048) |
| MBb63 | 0.086584 p = (0.000503) |
| MBb57 | 0.088857 p = (0.022) |
| MBb59 | 0.105447 p = (0.000379) |
| PS-2 | 0.117345 p = (0.032) |
| MBb53 | 0.124319 p = (0.014) |
| MBa4 | 0.130598 p = (0.032) |
| MBb33 | 0.131257 p = (0.037) |
| MBWhit44 | 0.136321 p = (0.028) |
| MBa10 | 0.136675 p = (0.018) |
| MBWhit1 | 0.14573 p = (0.029) |
| MBb56 | 0.149617 p = (0.002) |
| MBb74 | 0.163515 p = (0.006) |
| MBWhit47 | 0.164153 p = (0.022) |
| MBWhit12 | 0.180354 p = (0.004) |
| MBb52 | 0.196163 p = (0.008) |
| MBb31 | 0.210762 p = (0.000184) |
| PC-1 | 0.215889 p = (0.005) |

(“NS” indicates no statistically significant improvement relative to control;
“—” indicates not tested)

Scratch assays were also performed on keratinocytes. Keratinocytes were grown to confluency and scratch assays were performed and measured as above with fibroblasts. As shown in Table 8, MBb11 significantly increased the rate of keratinocyte migration.

TABLE 8
Keratinocyte scratch assay performed in replicate (N = 8).

| | 10 ng/ml | 100 ng/ml | 1000 ng/ml |
|---|---|---|---|
| | (% change | (% change | (% change |
| | relative | relative | relative |
| Peptide | to control) | to control) | to control) |
| MBb11 | — | — | 0.389156 (p = 0.037) |
| MBb5 | — | — | NS |
| MBm2 | NS | — | — |
| EA1 | — | — | NS |
| EA2 | — | NS | — |
| P2 + P3 | — | — | NS |

(“NS” indicates no statistically significant improvement relative to control;
“—” indicates not tested)

### Summary of in Fibroblast and Keratinocyte In Vitro Experiments

Table 9 below summarizes the results of the MFT, proliferation, fibroblast scratch, keratinocyte scratch, and inflammation assays. Shown are the number of peptides in which a significant effect was observed with the concentrations tested, and the range of efficacy with the concentrations tested.

TABLE 9
Summary of tested Peptides

| N = 8 | No. | % | Highest performance | |
|---|---|---|---|---|
| replicate experiments | of hits | of hits | (% change relative to control) | Range |
| Proliferation fibroblasts | 1 | 6.30% | 73% (p = 0.009) | — |
| Proliferation keratinocytes | 20 | 10% | 21.5% p = (0.005) | 7%-22% |
| N = 3 | No. | % | | |
| replicate experiments | of hits | of hits | Highest performance | Range |
| MFT | 46 | 22% | 75% p = (0.053) | 14%-75% |
| Proliferation fibroblasts | 8 | 4.90% | 117% (p = 0.056) | 29%-117% |
| Scratch fibroblast | 9 | 5.60% | 178% (p = 0.036) | 59%-202% |
| Scratch keratinocyte | 1 | 14% | 39% (p = 0.37) | — |
| Inflammation | 3 | 30% | 29% (p < 0.05) | 22-29% |

(“NS” indicates no statistically significant improvement relative to control;
“—” indicates not tested)

### Antimicrobial Effects of Peptides

To determine which peptides may be used to inhibit the growth of bacteria in tissue, the minimum inhibitory and bactericidal concentrations of exemplary peptides against both Gram-positive and Gram-negative bacteria were assessed. First MBb32 peptide was tested for antimicrobial effects, and in a separate set of experiments, Mbdol20, Mbdol15, and Mbdol23 were also tested for their antimicrobial effects.

The antimicrobial effects of the exemplary peptides were assessed in the following bacterial strains: Staphylococcus aureus: USA300, ST80, JE2, and MRSA col; Streptococcus pneumoniae: DCC1335 and D39; Klebsiella pneumoniae ATCC 10031; Pseudomonas aeruginosa ATCC 27853; and Acinetobacter baumannii ATCC 196062.

Cell culture: Cultures of each bacterial strain were prepared by inoculating on Muller Hinton Agar (MHA) medium. After overnight incubation at 37° C., the density of the bacterial cells was compared to the McFarland turbidity standard (0.5).

### Diffusion Agar Plate Method:

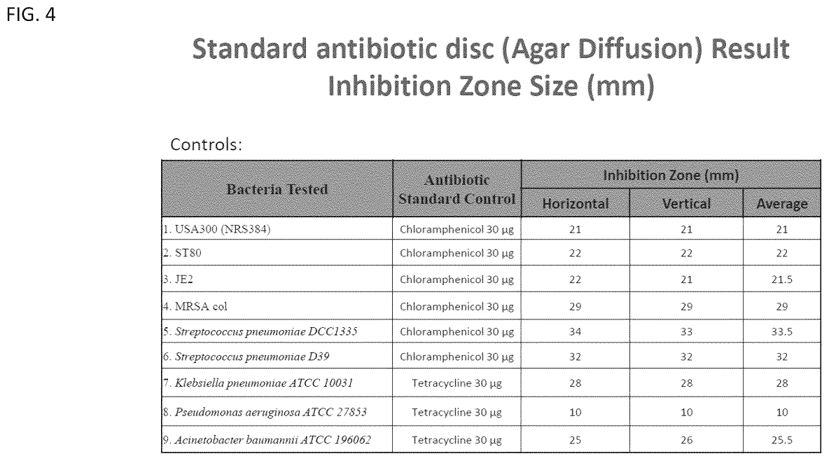

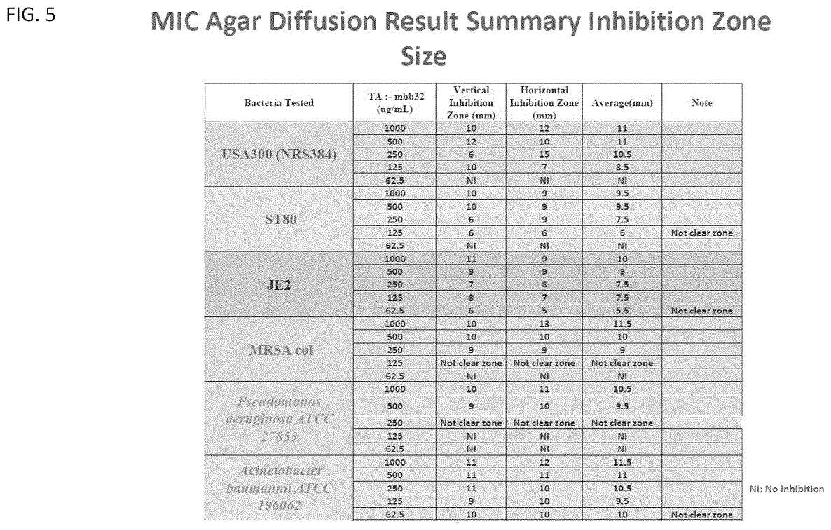

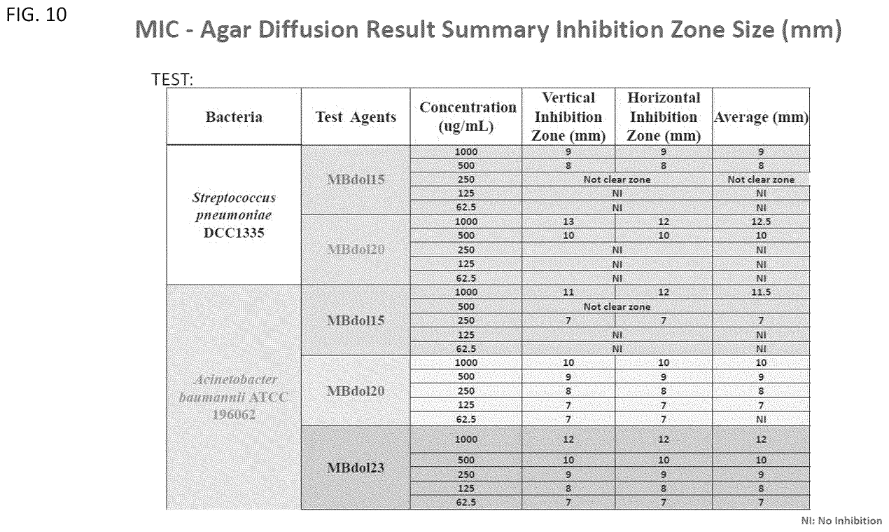

Plates were swabbed with McFarland 0.5 inoculum of each test bacteria. Ten-fold serial dilutions of the peptides were prepared and 20 μl of compound were dropped onto the MHA. Antibiotic disc served as a positive control and vehicle alone served as the negative control. Plates were incubated for 16-18 hours and assessed for antibacterial activity. Zones of inhibition were measured for each TA dilution and compared with the controls. FIG. 4 shows a control Table of the agar zone of inhibition with the antibiotics Chloramphenicol and Tetracycline and the bacterial strains tested in FIG. 5 and FIG. 10.

### Broth Microdilution Method:

Ten-fold serial dilution of the peptide along with controls were prepared in Mueller Hinton Broth (MHB) media in a total volume of 50 μl in 96 well plates. Additionally, 50 μl of the diluted standard inoculum (1:150 in MHB media) were added to each of the well plates. The plates were read at OD600 nm. MIC50 and MIC90 curves were calculated from the plate readout. Each sample was run in duplicate.

### Minimum Bactericidal Concentration:

The minimum bactericidal concentration (MBC) test determines the lowest concentration at which an antimicrobial agent will kill a particular microorganism. The MBC is determined using a series of steps, undertaken after a Minimum Inhibitory Concentration (MIC) test has been completed. o determine the MBC, the dilution representing the MIC and at least two of the more concentrated test product dilutions are plated and enumerated to determine viable CFU/ml. The minimum inhibitory concentration of the peptides was determined as the lowest concentration in which colony forming units (CFU) values were <5 colonies/10 μl of culture.

### Results:

FIG. 5 shows the agar zone of inhibition the MBb32 peptide in Staphylococcus aureus USA300 (NRS384), Staphylococcus aureus ST80, Staphylococcus aureus JE2, Staphylococcus aureus MRSA col, Pseudomonas aeruginosa ATCC 27853, and Acinetobacter baumannii ATCC 196062 and at concentrations ranging from 62.5 μg/ml to 1000 μg/ml. The data show that MBb32 was effective at different concentrations in inhibiting bacterial growth in all of the bacteria tested.

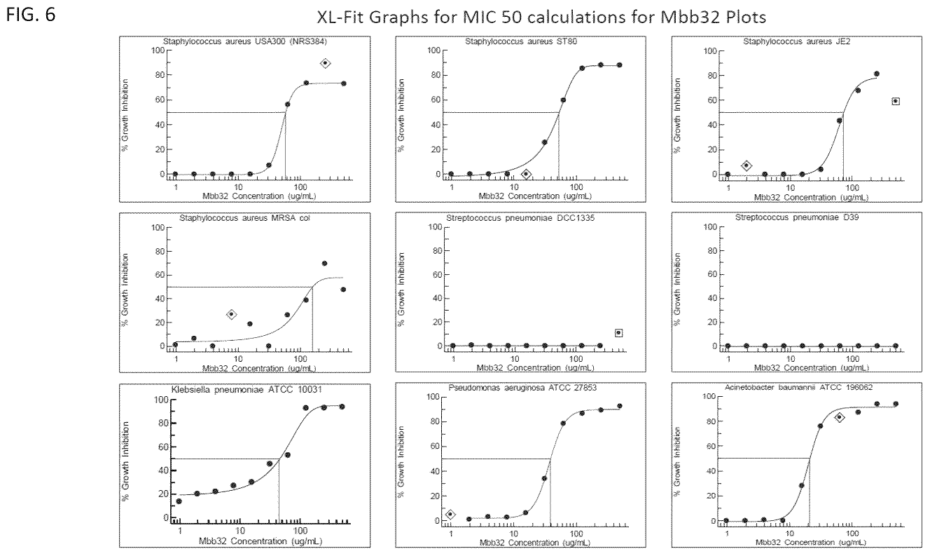

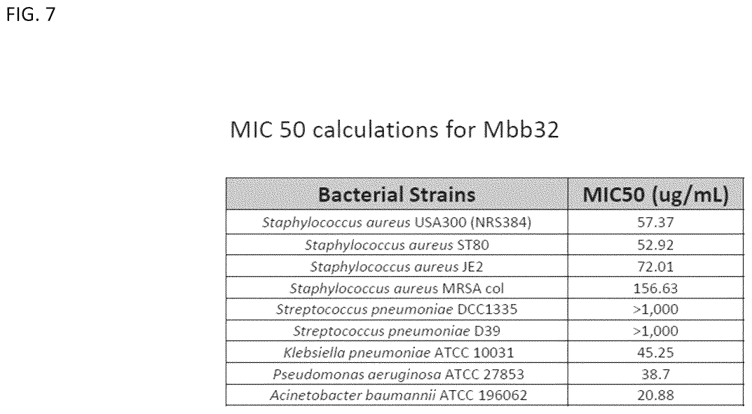

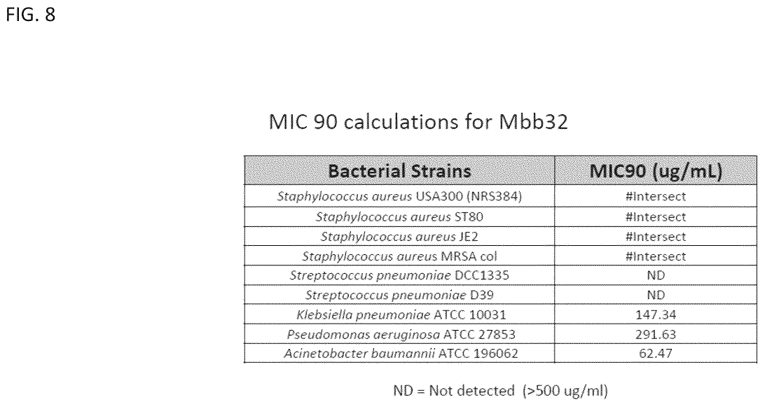

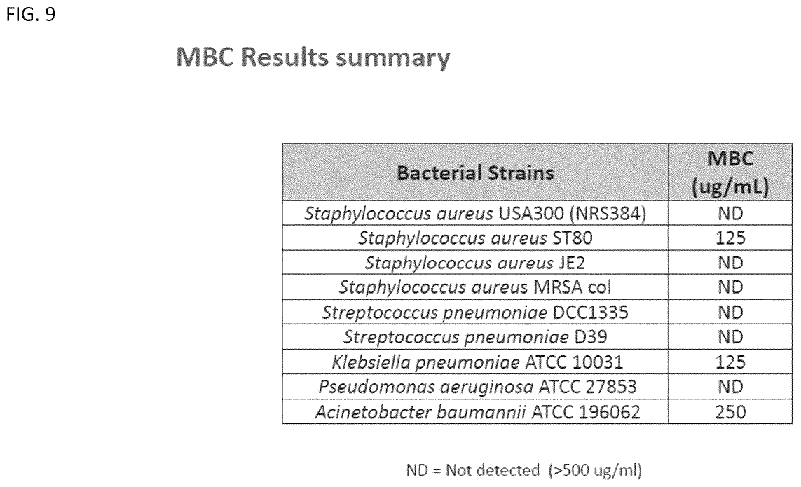

FIG. 6 shows MBb32 peptide concentration as a function of growth inhibition of bacteria in broth, and FIGS. 8 and 9 summarize the calculated MIC50 values as a function of the growth inhibition of bacteria of the MBb32 peptide. Notably MBb32 has the lowest MIC50 in Acinetobacter baumannii ATCC 196062, but also demonstrates a low MIC50 in the Staphylococcus aureus strains, including the MRSA col strain, as well as in Klebsiella pneumoniae ATCC 10031 and Pseudomonas aeruginosa ATCC 27853.

FIG. 9 shows the minimum bactericidal concentration (MBC) at which the MBb32 peptide will kill each bacterial strain on agar plates. The MBb32 peptide showed a bactericidal effect in Staphylococcus aureus ST80, Klebsiella pneumoniae ATCC 10031, and Acinetobacter baumannii ATCC 196062.

FIG. 10 shows the agar zone of inhibition of the MBdol15, and MBdol20, and MBdol23 peptides in Streptococcus pneumoniae DCC1335 and Acinetobacter baumannii ATCC 196062 at concentrations ranging from 62.5 μg/ml to 1000 μg/ml. The data show that MBdol15, MBdol20, and MBdol23 peptides were effective at different concentrations in inhibiting bacterial growth in each of the bacteria tested.

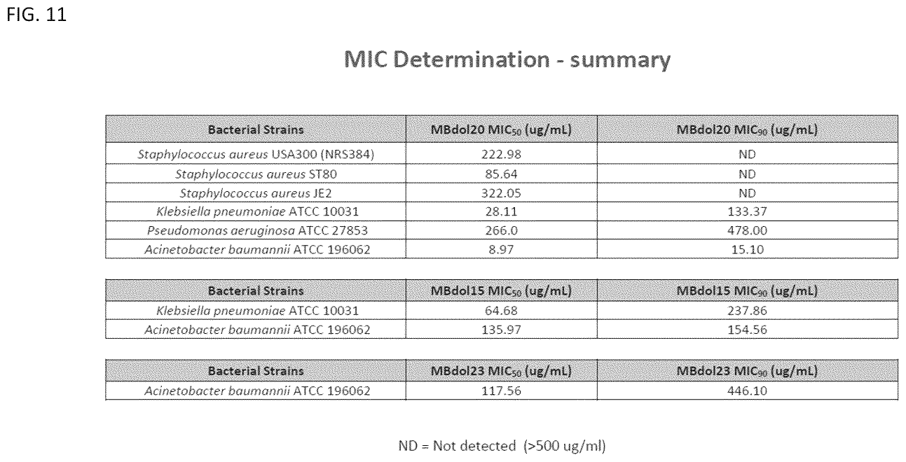

FIG. 11 summarizes the calculated MIC50 and MIC90 values for the MBdol15, and MBdol20, and MBdol23 peptides, as a function of the growth inhibition of bacteria in broth, in Staphylococcus aureus USA300 (NRS384), Staphylococcus aureus ST80, Staphylococcus aureus JE2, Klebsiella pneumoniae ATCC 10031, Pseudomonas aeruginosa ATCC 27853, and Acinetobacter baumannii ATCC 196062. The data show that Mbdol20 was most effective at inhibiting bacterial growth in Acinetobacter baumannii ATCC 196062.

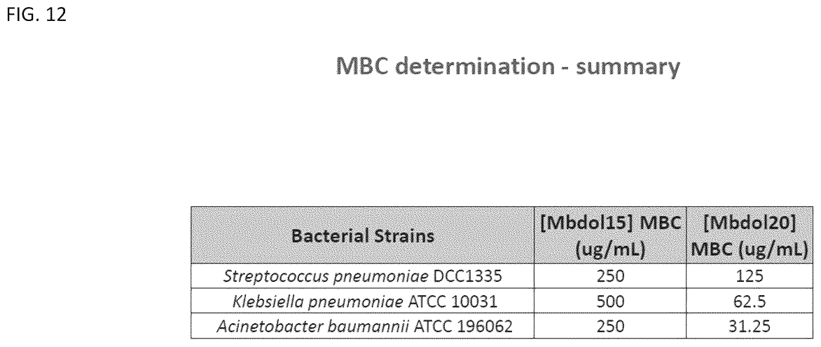

FIG. 12 shows the minimum bactericidal concentration (MBC) at which the MBdol15 and MBdo120 peptides will kill each of Streptococcus pneumoniae DCC1335, Klebsiella pneumoniae ATCC 10031, and Acinetobacter baumannii ATCC 196062 on agar plates.

### Example 2: Evaluating the Effects of Different Topical Products on Skin Condition Postdermabrasion

To determine the effects of exemplary peptides p1-p9 on tissue healing, a clinical study was performed that assessed transepidermal water loss (TEWL) and skin smoothness after dermabrasion.

### Methods:

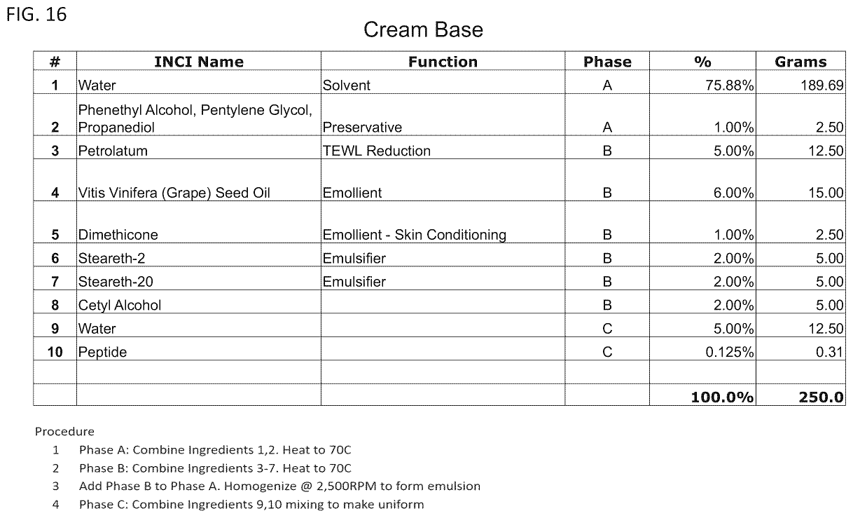

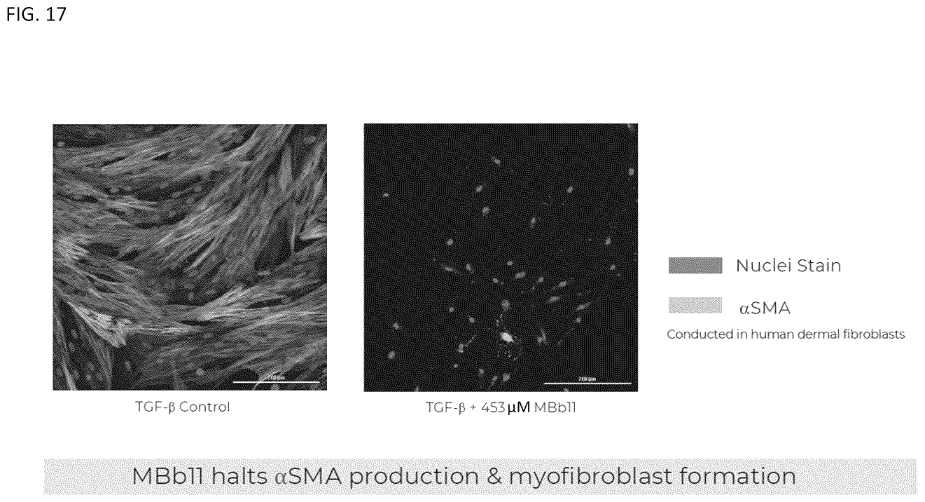

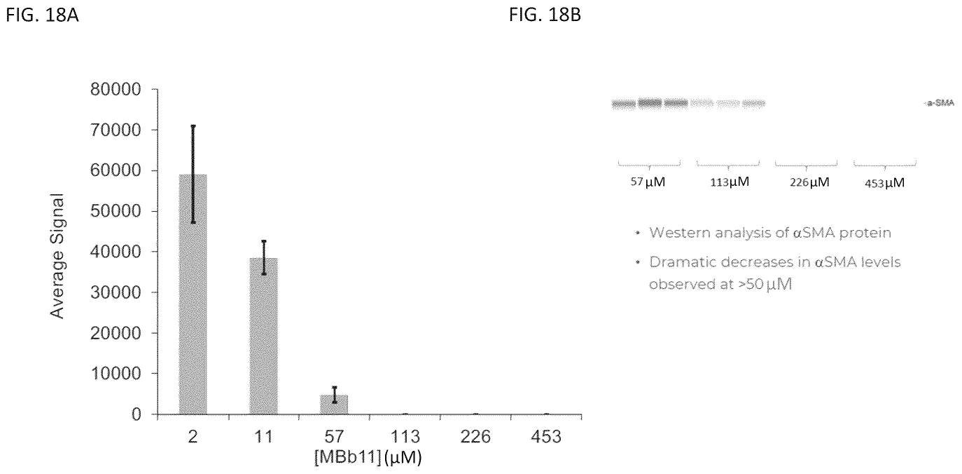

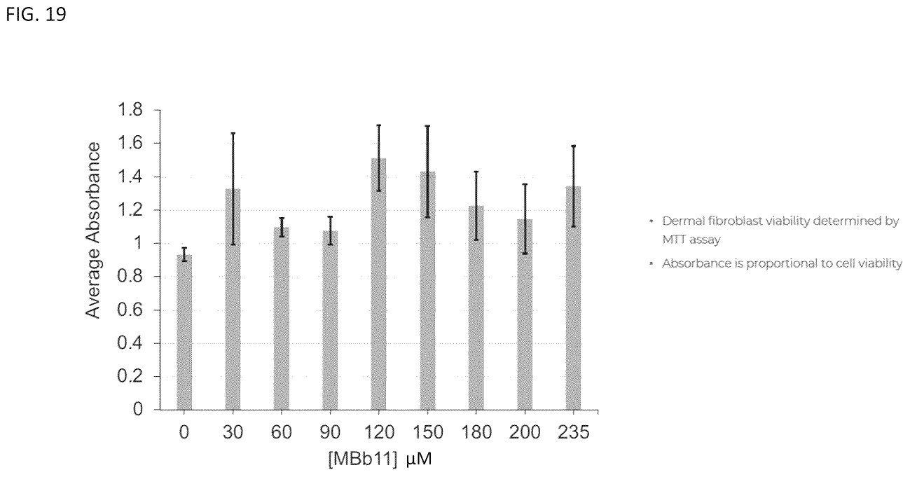

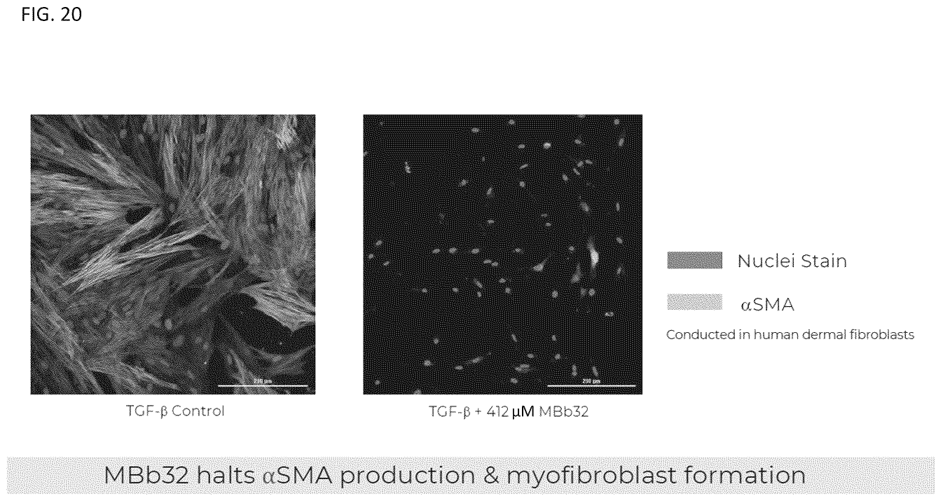

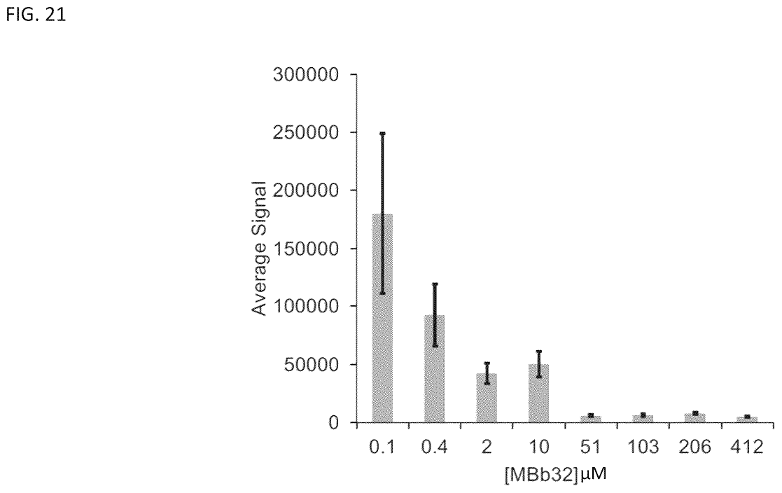

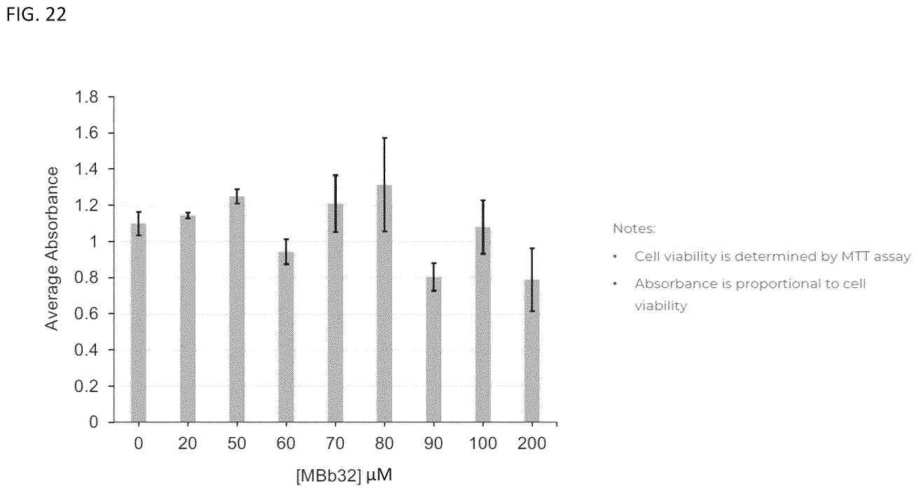

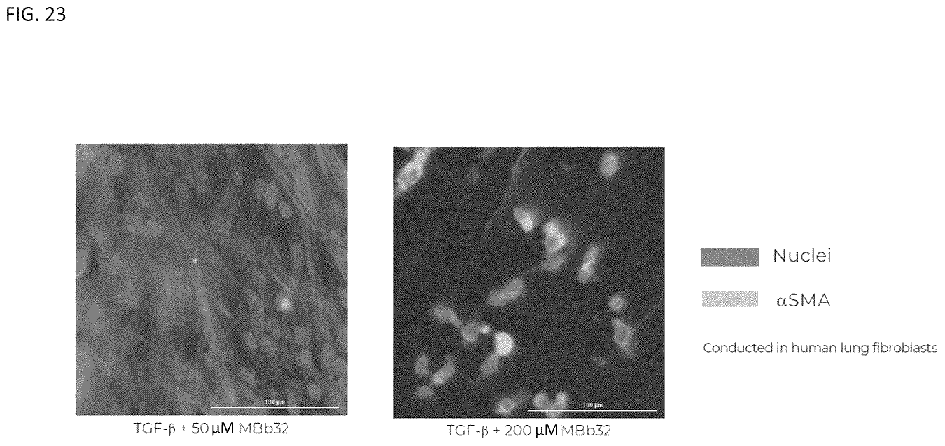

The study subjects received the support products including p1-p9, formulated in the vehicle cream base provided in FIG. 16. This exemplary vehicle cream has a pH of 5.0, no odor and is white in color.

P1-P3, and P6 are single peptides. P4 is a combination of peptides: PA-1, FA-1, P1, and EA-4. P5 is a combination of peptides: PC-1, P3, P2, and EC-1. P7 is a combination of peptides: PH-1, PA-3, PC-1, EH-2, EA-4, P1, P2, EC-1, and P3. P8 is a combination of peptides: PH-1, PA-3, PC-1, EH-2, EA-4, P2, P1, EC-1, P3, EA-1, EA-2, PS-2, and ES-1.

After 8 hours (T8h) of first product application, the subjects returned to the Institute, acclimated for 15 minutes and new Vapometer® to measure TEWL and Visioscan® VC 20Plus to measure skin smoothness was performed. After 1 (T1d), 2 (T2d) and 3 (T3d) days the subjects returned to the Institute, acclimated for 15 minutes and new measurements were performed. At time-points T1d and T2d new product application was performed by the technician on each test site. A total of 49 study subjects were included in the study and a total of 41 completed the study. The study included post-menopausal female subjects, aged between 50 and 65 years old (mean age: 59 years old), presenting Fitzpatrick Skin Types II-V.

### Vapometer® Measurements

The TEWL measurements were performed by using the Vapometer® device through a measurement probe. The readings were taken by applying the probe to the test sites, by resting it against the skin.

The measurements principle is diffusion, which indicates the mass of water per cm² that is transported in a given period of time, that is, the reading indicates the degree of water evaporation on the skin surface based on the sensitivity of hygro-sensors. The scale used is g/h/m², in which greater values of reading indicate higher evaporation. The decrease in TEWL values indicates the decrease in transepidermal water loss and the improvement of the skin barrier and no differences in TEWL values indicates no alteration of the skin barrier.

### Visioscan Measurements

Skin texture images were performed using the Visioscan® VC 20Plus, which allows taking pictures of the skin through magnifying lenses. The images enable a view of the skin topography, texture, dryness, desquamation, blemishes, pores, and cuticles.

### Results:

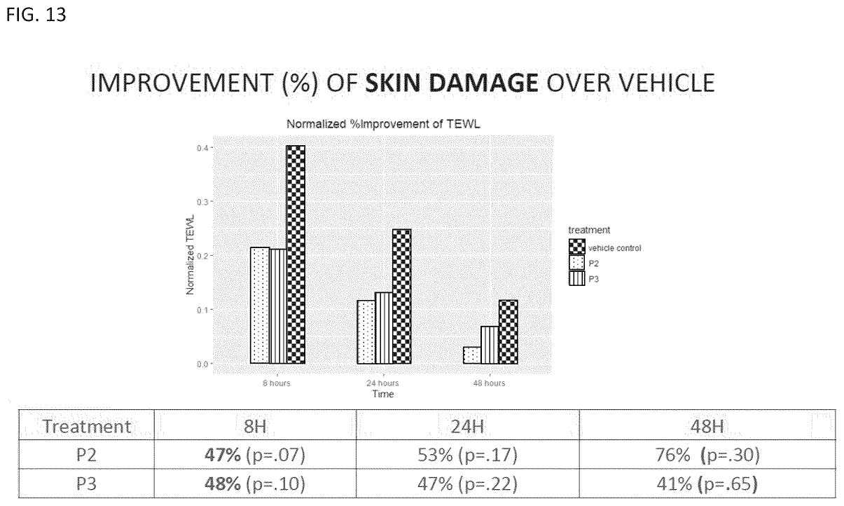

Out of all the peptides, P2 and P3 demonstrated the most promising skin smoothness results, as assessed by Visioscan, and as shown in FIGS. 16A and 16B. Further, P2 and P3 also showed the most promising results in skin barrier restoration, as assessed by the TEWL measurements, and as demonstrated in FIG. 13.

### Example 3: Erythema Evaluation of Peptides P2, P3, and P2+P3

To determine the effects of the peptides on skin redness, the peptides P2, P3, and the combination of P2+P3 were tested at different concentration levels and subject's skin redness (erythema) was measured post-dermabrasion and 2, 4, 8, 12, 24, 48 and 72 hours post-application.

The subjects were 30 post-menopausal female subjects, aged 50-65 years or younger, if post-menopausal. Enrolled subjects were to have Fitzpatrick Skin Types II-V.

### Chromameter® Measurements

At baseline (post-dermabrasion) and 2, 4, 8, 12, 24, 48 and 72 hours post-application, skin redness was measured on 4 skin sites using the Chromameter® (Konica Minolta, Ramsey NJ). In order to determine any changes in skin redness, the a* value was analyzed. The a* value measures redness/erythema in the skin. A decrease in the a* value corresponded to a whitening/improvement (less red) effect and an increase in the a*value represented a darkening/worsening (redder) effect

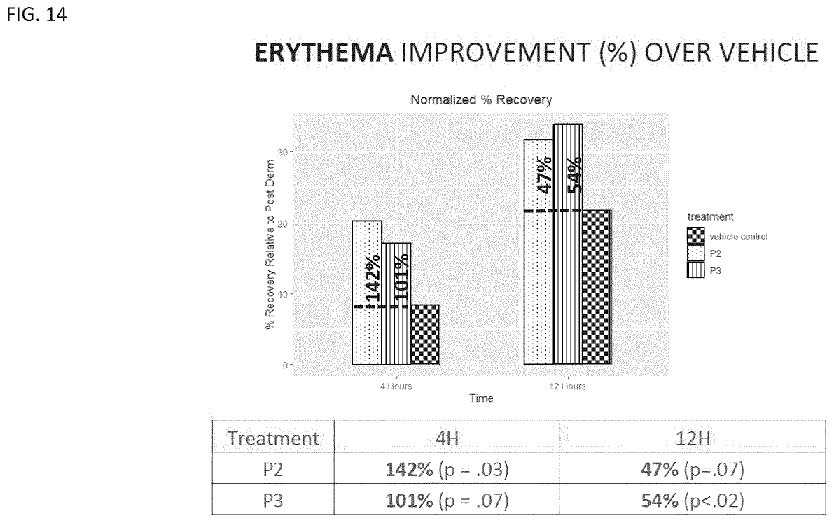

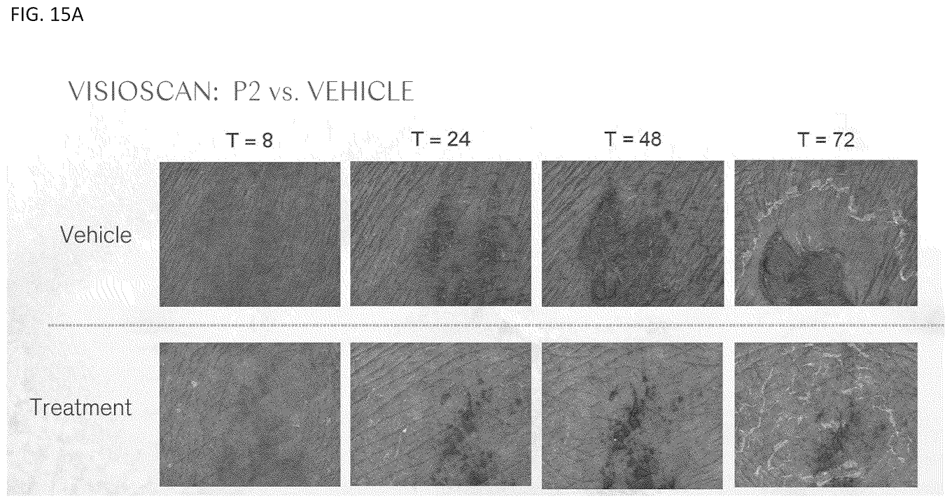

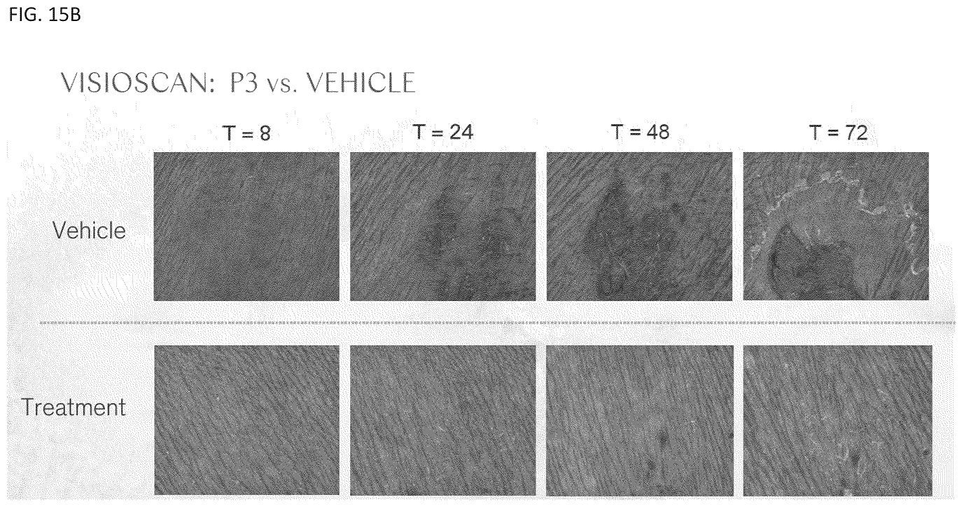

Results: As shown in FIG. 14, each of P2 and P3 improve skin redness (erythema) post-dermabrasion at both 4 hours and 12 hours after-application.

### Example 4: Dermal Penetration Assays

Determining the depth of penetration of peptides is crucial in predicting their efficacy. Actively proliferating keratinocytes reside in the basal layer of the epidermis, while proliferating fibroblasts reside within the dermal layer. Therefore, determining which layers the peptides are able to reach will allow the determination of affected cell types.

An example assay to determine the depth of penetration of the peptides is a tape stripping assay, in which peptides are added to a human subject. After application, tape stripping is performed on the subject. Each of the layers of the skin is tape stripped and each layer is sent for an HPLC-MS analysis. The peptide is identified in each of the stripped layers to determine the depth of peptide penetration

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A method of synthesizing a peptide for an anti-fibrotic treatment comprising:
a. selecting a peptide sequence from a regenerative protein sequence from a regenerative species; and
b. modifying the peptide sequence to improve one or more of cross-species homology, tissue penetration, peptide stability, and/or affinity to human proteases; and
c. synthesizing the peptide;
wherein the peptide regulates one or more of: myofibroblast formation, bacterial infection, inflammation, cell migration, extracellular matrix production, keratin production, or cell proliferation.

2. The method of claim 1, wherein the regenerative species is selected from axolotl, spiny mouse, starfish, planarian, and zebrafish.

3. The method of claim 1, wherein the modifying to improve cross-species homology includes adding one or more amino acid residues from a homologous sequence at the C-terminus or N-terminus of the peptide fragment or modifying the amino acid sequence of the peptide to increase the cross-species sequence homology by at least 20%.

4. The method of claim 1, wherein the peptide sequence selected from the regenerative protein sequence is between about 5 to about 25 amino acids in length.

5. The method of claim 1, wherein the regenerative protein sequence from a regenerative species is one or more of the sequences of Table 1.

6. The method of claim 1, wherein the peptide comprises an amino acid sequence of Table 2, or an amino acid sequence with at least 70% sequence identity thereto, and wherein the peptide comprises at least one modification relative to the wild-type regenerative protein.

7. The peptide of claim 6, wherein the peptide comprises the amino acid sequence of P2, or a sequence with at least 70% sequence identity thereto, with at least one modification relative to the wild-type sequence in the Epiplakin protein; or a sequence of P3, or a sequence with at least 70% sequence identity thereto, with at least one modification relative to the wild-type sequence in the FGF-Binding protein.

8. A composition comprising one or more peptides of claim 6, and one or more adjuvants or excipients.

9. The composition of claim 8, wherein the composition is formulated together with a skin protectant, wound healing, or acne treatment monograph to produce a peptide formulation.

10. The formulation of claim 9, wherein the peptide formulation is applied topically as a serum, gel, lotion, cream, ointment, mask, peel, or spray.

11. A modified regenerative protein derived from a wild-type regenerative protein of a regenerative species, comprising at least one modification relative to the wild-type protein sequence, wherein the at least one modification improves one or more of human sequence homology, tissue penetration, peptide stability, and/or affinity to human proteases, and wherein the peptide regulates one or more of: myofibroblast formation, bacterial infection, inflammation, cell migration, extracellular matrix production, keratin production, or cell proliferation.

12. A method of selecting one or more peptides from a subset of peptides for use in an anti-fibrotic treatment comprising:
a. performing one or more assays on the peptides;
b. comparing a result of the one or more assays of peptides relative to a control, wherein the one or more assays are selected from: a myofibroblast transition assay (MFT), a scratch assay, a proliferation assay, an inflammation assay, and an anti-microbial assay; and
c. selecting the peptide for use in an anti-fibrotic treatment from the subset of peptides when the result of the one or more assays for the one or more given peptide is at least 40% greater than the control, or when the results of two or more assays for the one or more given peptides are both at least 25% greater than the control.

13. The method of claim 12, comprising selecting the one or more peptides for use in an anti-fibrotic treatment when:
a. the result of the MFT assay is at least 25%, at least 30%, at least 35%, at least 40%, at least 45%, at least 50%, at least 55%, at least 60%, at least 65%, at least 70%, at least 75%, or at least 80% greater than the control;
b. the result of the scratch assay is at least 35%, at least 40%, at least 45%, or at least 50% greater than the control when performed on keratinocytes;
c. the result of the scratch assay is at least 40%, at least 60%, at least 80%, at least 100%, at least 120%, at least 140%, at least 160%, or at least 180% greater than the control when performed on fibroblasts;
d. the result of the proliferation assay is at least 40%, at least 60%, at least 80%, or at least 100% greater than the control when performed on fibroblasts; or
e. the result of the proliferation assay is at least 35%, at least 40%, at least 45%, or at least 50% greater than the control when performed on keratinocytes.

14. The method of claim 13, comprising selecting the one or more peptides for use in an anti-fibrotic treatment when the result of the inflammation assay is at least 20% greater than the control, or the result of the anti-microbial assay is at least 20% greater than the control.

15. A method of treatment comprising:
applying a formulation comprising a peptide to an area to be treated, wherein the peptide is derived from a regenerative species regenerative protein, and wherein the peptide is modified to include one or more amino acid residues from the homologous human sequence,
wherein the peptide regulates one or more of: myofibrolast differentiation, bacterial infection, inflammation, cell migration, or cell proliferation.

16. The method of claim 15, wherein the method of treatment comprises an anti-fibrotic treatment that reduces fibrosis.

17. The method of claim 16, wherein the anti-fibrotic treatment treats systemic scleroderma, localized scleroderma (i.e., morphea), eosinophilic fasciitis (i.e., Shulman's syndrome), lipodermatosclerosis, nephrogenic systemic fibrosis (NSF), scleredema, scleromyxedema, diffuse cutaneous systemic sclerosis, chronic graft-versus-host Disease (cGVHD) with skin involvement, dermatofibrosarcoma protuberans (DFSP), sclerotic chronic graft-versus-host disease (cGVHD), lichen sclerosus, calciphylaxis (calcific uremic arteriolopathy), acrodermatitis chronica atrophicans, poikiloderma of civatte, epidermolysis bullosa, lichen planus, keloids, hypertrophic scarring, or necrobiosis lipoidica.

18. The method of claim 17, wherein the anti-fibrotic treatment treats epidermolysis bullosa, localized scleroderma, or necrobiosis lipoidica.

19. The method of claim 17, wherein the anti-fibrotic treatment treats keloids, hypertrophic scarring, systemic scleroderma, lichen planus, lichen sclerosus, or lipodermatosclerosis.

20. The method of claim 16, wherein the anti-fibrotic treatment treats fibrosis associated with or caused by a disease or disorder.

21. The method of claim 20, wherein the disease is cancer, a metabolic disorder, or a genetic disorder.

22. The method of claim 16, wherein the anti-fibrotic treatment treats fibrosis associated with or caused by surgery, injury, infection, substance use, or exposure to allergens.

23. The method of claim 16, wherein the anti-fibrotic treatment treats fibrosis associated with or caused by exposure to allergens.

24. The method of claim 16, wherein the anti-fibrotic treatment treats liver fibrosis, lung fibrosis, cardiac fibrosis, kidney fibrosis, pancreatic fibrosis, intestinal fibrosis, ocular fibrosis, bone marrow fibrosis, uterine fibrosis, and/or ovarian fibrosis.

25. The method of claim 16, wherein the anti-fibrotic treatment comprises scar prevention or reduction, post-surgical wound healing, tissue or organ generation, chronic wound healing, epithelial disorders, age-related skin impact, treatment of burns, reduction of inflammation, reduction of infection, or combinations thereof.

26. The method of claim 16, wherein the anti-fibrotic treatment reduces scar formation.

27. The method of claim 26, wherein the scar formation is post-surgical.

28. The method of claim 16, wherein the anti-fibrotic treatment treats tissue or organ generation.

29. The method of claim 26, wherein the anti-fibrotic treatment reduces scarring associated with pressure ulcers, diabetic ulcers, foot ulcers, burns, epithelial disorders, psoriasis, dermatitis, rosacea, acne, or diaper rash.

30. The method of claim 16, wherein the anti-fibrotic treatment reduces age-related skin impact.


SEQUENCE LISTING (96 sequences)

SEQ ID NO: 1 - protein, 12 aa, synthetic construct
GLRKRLRKFRNK

SEQ ID NO: 2 - protein, 690 aa, synthetic construct
GGHRAAGSLSPIQERTRRTREEQNWQTKMRTSTVLHAAGFLLRLLLVNVLVFQDTIAAPSPIIKFPGDSTPKTEKELAVQYLNKFYGCPKEKCNLFVLKDTLKKMQKFFGLPETGELDQNTIETMKKPRCGNPDVANYNFFPRKPKWEKNHITYRIVGYTTDLDTETVDDAFARAFKVWSDVTPLDFSRLNDGDADIMINFGRWEHGDGYPFDGKDGLLAHAFAPGQGIGGDSHFDDDEHWTLGDGQVVKVKFGNADGEFCKFPFLFGDKEYNSCTDAGRSDGFLWCSTTYNFDSDKKYGFCPHELLFTLAGNADGEPCKFPFKFQGTSYDSCTTEGRTDGYRWCATTADYDTDKKYGFCPETALSTVGGNSEGSPCVFPFTFLGNKYDQCTSSGRSDGKLWCASTSNYDDDRKWGFCPDQGYSLFLVAAHEFGHALGLEHSDDPGALMAPIYTYTKNFKLSQDDVSGIQELYGGTSEKPGPGPSPTLGPVTPELCKKDITFDALSQIRGEIFLFKDRFIWRTVSPRTRPSGPLLVATFWSEVPDKIDAVYEAPQEEKTVFFAGNEYWIYSASQLERGYPKRLTALGLSDDVKHVDAAFNWSKNKKTYIFSGDKFWRYNEVKKKMDPGFPKLIADSWTGVPDDLDAVLDHSGSGYTYFIKDKYYLKLENESLKIVQIGNMKNDWLGCRPS

SEQ ID NO: 3 - protein, 660 aa, synthetic construct
MEALMARGALTGPLRALCLLGCLLSHAAAAPSPIIKFPGDVAPKTDKELAVQYLNTFYGCPKESCNLFVLKDTLKKMQKFFGLPQTGDLDQNTIETMRKPRCGNPDVANYNFFPRKPKWDKNQITYRIIGYTPDLDPETVDDAFARAFQVWSDVTPLRFSRIHDGEADIMINFGRWEHGDGYPFDGKDGLLAHAFAPGTGVGGDSHFDDDELWTLGEGQVVRVKYGNADGEYCKFPFLFNGKEYNSCTDTGRSDGFLWCSTTYNFEKDGKYGFCPHEALFTMGGNAEGQPCKFPFRFQGTSYDSCTTEGRTDGYRWCGTTEDYDRDKKYGFCPETAMSTVGGNSEGAPCVFPFTFLGNKYESCTSAGRSDGKMWCATTANYDDDRKWGFCPDQGYSLFLVAAHEFGHAMGLEHSQDPGALMAPIYTYTKNFRLSQDDIKGIQELYGASPDIDLGTGPTPTLGPVTPEICKQDIVFDGIAQIRGEIFFFKDRFIWRTVTPRDKPMGPLLVATFWPELPEKIDAVYEAPQEEKAVFFAGNEYWIYSASTLERGYPKPLTSLGLPPDVQRVDAAFNWSKNKKTYIFAGDKFWRYNEVKKKMDPGFPKLIADAWNAIPDNLDAVVDLQGGGHSYFFKGAYYLKLENQSLKSVKFGSIKSDWLGC

SEQ ID NO: 4 - protein, 316 aa, synthetic construct
MDAYWAFLLAPVFLFYIAAPYIRKFVGGGVCKSTAKLHGKVVVITGANTGIGKETASDLARRGARVVIACRDVAKGEAAASKIRAETGNEQVVVRKLDLADTKSVGEFAERMRKEEERIHILINNAGVMMCPYSKTVDGFEMQLGVNHLGHFLLTYLLLDVLKKSAPARIVNVSSLAHHFGRIKFEDLQCEKSYQSALAYCRSKLANILFTRELAKRLKGTQVTVNALHPGSVRSELVRHSFLLSKAWTLCSFFIKTPKEGAQTNIYCAVAEELESVSGKYFSDCSPAYVSSQGRNDETARKLWDVSCRLLGIQWN

SEQ ID NO: 5 - protein, 316 aa, synthetic construct
MLVTLGLLTSFFSFLYMVAPSIRKFFAGGVCRTNVQLPGKVVVITGANTGIGKETARELASRGARVYIACRDVLKGESAASEIRVDTKNSQVLVRKLDLSDTKSIRAFAEGFLAEEKQLHILINNAGVMMCPYSKTADGFETHLGVNHLGHFLLTYLLLERLKVSAPARVVNVSSVAHHIGKIPFHDLQSEKRYSRGFAYCHSKLANVLFTRELAKRLQGTGVTTYAVHPGVVRSELVRHSSLLCLLWRLFSPFVKTAREGAQTSLHCALAEGLEPLSGKYFSDCKRTWVSPRARNNKTAERLWNVSCELLGIRWE

SEQ ID NO: 6 - protein, 5 aa, synthetic construct
YCRSK

SEQ ID NO: 7 - protein, 7 aa, synthetic construct
FAYCRSK

SEQ ID NO: 8 - protein, 19 aa, synthetic construct
FAYCRSKGLRKRLRKFRNK

SEQ ID NO: 9 - protein, 17 aa, synthetic construct
EYENPGLRKRLRKFRNK

SEQ ID NO: 10 - protein, 18 aa, synthetic construct
QFCGPCGLRKRLRKFRNK

SEQ ID NO: 11 - protein, 18 aa, synthetic construct
DPCPLCGLRKRLRKFRNK

SEQ ID NO: 12 - protein, 5 aa, synthetic construct
ERDLK

SEQ ID NO: 13 - protein, 5 aa, synthetic construct
EEIDA

SEQ ID NO: 14 - protein, 17 aa, synthetic construct
EELTRGLRKRLRKFRNK

SEQ ID NO: 15 - protein, 7 aa, synthetic construct
AVHELGH

SEQ ID NO: 16 - protein, 13 aa, synthetic construct
IAHELGHNLGMSH

SEQ ID NO: 17 - protein, 5 aa, synthetic construct
PKPQP

SEQ ID NO: 18 - protein, 7 aa, synthetic construct
IAHELGH

SEQ ID NO: 19 - protein, 17 aa, synthetic construct
FVDADGLRKRLRKFRNK

SEQ ID NO: 20 - protein, 17 aa, synthetic construct
VGDFDGLRKRLRKFRNK

SEQ ID NO: 21 - protein, 18 aa, synthetic construct
NTCHQCGLRKRLRKFRNK

SEQ ID NO: 22 - protein, 18 aa, synthetic construct
GESKGLGLRKRLRKFRNK

SEQ ID NO: 23 - protein, 17 aa, synthetic construct
ENIKKGLRKRLRKFRNK

SEQ ID NO: 24 - protein, 6 aa, synthetic construct
LPRDER

SEQ ID NO: 25 - protein, 17 aa, synthetic construct
CGGLPGLRKRLRKFRNK

SEQ ID NO: 26 - protein, 17 aa, synthetic construct
PPSILGLRKRLRKFRNK

SEQ ID NO: 27 - protein, 5 aa, synthetic construct
RVIWA

SEQ ID NO: 28 - protein, 5 aa, synthetic construct
ASCRC

SEQ ID NO: 29 - protein, 17 aa, synthetic construct
SSSGLGLRKRLRKFRNK

SEQ ID NO: 30 - protein, 19 aa, synthetic construct
SAYGVSKGLRKRLRKFRNK

SEQ ID NO: 31 - protein, 17 aa, synthetic construct
AHSDLGLRKRLRKFRNK

SEQ ID NO: 32 - protein, 6 aa, synthetic construct
VIGKGG

SEQ ID NO: 33 - protein, 17 aa, synthetic construct
PRGQQGLRKRLRKFRNK

SEQ ID NO: 34 - protein, 20 aa, synthetic construct
KHFDSPRGGLRKRLRKFRNK

SEQ ID NO: 35 - protein, 17 aa, synthetic construct
TSRDEGLRKRLRKFRNK

SEQ ID NO: 36 - protein, 17 aa, synthetic construct
QLDPDGLRKRLRKFRNK

SEQ ID NO: 37 - protein, 18 aa, synthetic construct
VHEIQRGLRKRLRKFRNK

SEQ ID NO: 38 - protein, 18 aa, synthetic construct
SVTWIIGLRKRLRKFRNK

SEQ ID NO: 39 - protein, 18 aa, synthetic construct
ASLHGNGLRKRLRKFRNK

SEQ ID NO: 40 - protein, 17 aa, synthetic construct
AQYNPGLRKRLRKFRNK

SEQ ID NO: 41 - protein, 5 aa, synthetic construct
WVSGL

SEQ ID NO: 42 - protein, 17 aa, synthetic construct
EKKNPGLRKRLRKFRNK

SEQ ID NO: 43 - protein, 5 aa, synthetic construct
RWMAP

SEQ ID NO: 44 - protein, 6 aa, synthetic construct
IGPGLP

SEQ ID NO: 45 - protein, 15 aa, synthetic construct
MDQGYPKETVDDFPG

SEQ ID NO: 46 - protein, 7 aa, synthetic construct
RNPDSPQ

SEQ ID NO: 47 - protein, 7 aa, synthetic construct
HNGILID

SEQ ID NO: 48 - protein, 10 aa, synthetic construct
HIWAVVPCIR

SEQ ID NO: 49 - protein, 10 aa, synthetic construct
VLFEDVIAEP

SEQ ID NO: 50 - protein, 10 aa, synthetic construct
QLFEDVIAEP

SEQ ID NO: 51 - protein, 7 aa, synthetic construct
AKEVEKH

SEQ ID NO: 52 - protein, 7 aa, synthetic construct
VYFVIKT

SEQ ID NO: 53 - protein, 41 aa, synthetic construct
CDADKKDYCLFGRCQYIVELETFRCRCQKGYYGERCVYLEL

SEQ ID NO: 54 - protein, 46 aa, synthetic construct
IRTLDCDADKKDYCLFGRCQYIVELETFRCRCQKGYYGERCVYLEL

SEQ ID NO: 55 - protein, 6 aa, synthetic construct
DCRICS

SEQ ID NO: 56 - protein, 10 aa, synthetic construct
IDFEDVIAEP

SEQ ID NO: 57 - protein, 8 aa, synthetic construct
FEDVIAEP

SEQ ID NO: 58 - protein, 7 aa, synthetic construct
GYRCSKG

SEQ ID NO: 59 - protein, 7 aa, synthetic construct
ITCPHCQ

SEQ ID NO: 60 - protein, 8 aa, synthetic construct
PSMELVLW

SEQ ID NO: 61 - protein, 5 aa, synthetic construct
ERCVY

SEQ ID NO: 62 - protein, 14 aa, synthetic construct
AATHEFGHSLGLSH

SEQ ID NO: 63 - protein, 14 aa, synthetic construct
VAAHELGHSLGMGH

SEQ ID NO: 64 - protein, 15 aa, synthetic construct
LVAAHEVGHALGLGH

SEQ ID NO: 65 - protein, 10 aa, synthetic construct
QRLLEAQAAT

SEQ ID NO: 66 - protein, 8 aa, synthetic construct
SKPAKAPE

SEQ ID NO: 67 - protein, 10 aa, synthetic construct
LKEKKPAKEK

SEQ ID NO: 68 - protein, 30 aa, synthetic construct
SDAAVDTSSEITTKDLKEKKEVVEEAENGR

SEQ ID NO: 69 - protein, 15 aa, synthetic construct
FFLRIHPDGRVDGVR

SEQ ID NO: 70 - protein, 7 aa, synthetic construct
FRSCDLR

SEQ ID NO: 71 - protein, 6 aa, synthetic construct
RSCDLR

SEQ ID NO: 72 - protein, 8 aa, synthetic construct
CFRSCDLR

SEQ ID NO: 73 - protein, 12 aa, synthetic construct
CINTEGGYVCRC

SEQ ID NO: 74 - protein, 16 aa, synthetic construct
CMCAEGYALSRDRKYC

SEQ ID NO: 75 - protein, 14 aa, synthetic construct
FFLRIHPDGRVDGV

SEQ ID NO: 76 - protein, 6 aa, synthetic construct
GDIRVR

SEQ ID NO: 77 - protein, 6 aa, synthetic construct
KTHPGG

SEQ ID NO: 78 - protein, 16 aa, synthetic construct
IGERCQYRDLKWWELR

SEQ ID NO: 79 - protein, 16 aa, synthetic construct
DVLDKRLFWIQYNREG

SEQ ID NO: 80 - protein, 19 aa, synthetic construct
VCDEPKDQTVVGPALAAYR

SEQ ID NO: 81 - protein, 19 aa, synthetic construct
PHKGLFCHFGSPANRKIGV

SEQ ID NO: 82 - protein, 9 aa, synthetic construct
IVGLGPWLG

SEQ ID NO: 83 - protein, 12 aa, synthetic construct
VGLWKACTARGC

SEQ ID NO: 84 - protein, 15 aa, synthetic construct
GFWDILWHIPHTIKG

SEQ ID NO: 85 - protein, 17 aa, synthetic construct
SLSRTLWAWLPRPCKSN

SEQ ID NO: 86 - protein, 18 aa, synthetic construct
TFMSHLFCCVQKRCDCSF

SEQ ID NO: 87 - protein, 13 aa, synthetic construct
VSITEAMHRNLVD

SEQ ID NO: 88 - protein, 36 aa, synthetic construct
ADAAVENAVAPAESPVKDLKEKKEVVEEPAKEKAEN

SEQ ID NO: 89 - protein, 39 aa, synthetic construct
ECVEDVEAIDQRKVAEEYCGESWSSFCTFFFSMLQNKKC

SEQ ID NO: 90 - protein, 5 aa, synthetic construct
ERCVY

SEQ ID NO: 91 - protein, 15 aa, synthetic construct
CQKGYYGVRCEHLEL

SEQ ID NO: 92 - protein, 5 aa, synthetic construct
LKEKK

SEQ ID NO: 93 - protein, 5 aa, synthetic construct
PAKEK

SEQ ID NO: 94 - protein, 5 aa, synthetic construct
VRCEH

SEQ ID NO: 95 - protein, 41 aa, synthetic construct
CSSDMNGYCLHGQCIYLVDMSQNYCRCEVGYTGMRCEHFFL

SEQ ID NO: 96 - protein, 7 aa, synthetic construct
AAHEFGH